(19) United States
(12) Patent Application Publication
(10) Pub. No.: US 2023/0038748 A1
(43) Pub. Date: Feb. 9, 2023

(54) ASSAY FOR PROGNOSIS OF COVID-19 DISEASE

(71) Applicants: EM SCIENTIFIC LTD., London (GB); CHARITÉ - UNIVERSITÄTSMEDIZIN BERLIN, Berlin (DE)

(72) Inventors: Ernestas SIRKA, London (GB); Adam CRYAR, London (GB); Markus RALSER, Berlin (DE); Johannes HARTL, Berlin (DE); Ziyue WANG, Berlin (DE); Michael MÜLLEDER, Berlin (DE); Vadim DEMICHEV, Berlin (DE)

(21) Appl. No.: 17/685,756

(22) Filed: Mar. 3, 2022

Related U.S. Application Data
(60) Provisional application No. 63/283,787, filed on Nov. 29, 2021.
(60) Provisional application No. 63/156,291, filed on Mar. 3, 2021.

(51) Int. Cl. G01N 33/569; G01N 33/68

(52) U.S. Cl. CPC G01N 33/56983; G01N 33/6848; G01N 2800/60

(57) ABSTRACT

The present invention relates to a method for predicting and monitoring the severity of COVID-19 disease following infection of a subject with the SARS-CoV-2 virus. It also relates to a method for the treatment of a subject with COVID-19 disease. It also relates to kits for use in the methods of the invention.

### CROSS-REFERENCE TO RELATED APPLICATIONS

This application claims priority to U.S. Provisional Application No. 63/156,291, filed Mar. 3, 2021, and to U.S. Provisional Application No. 63/283,787, filed Nov. 29, 2021. The disclosures of the prior applications are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Mar. 3, 2022, is named P140814US02_Sequence_listing.txt and is 27,841 bytes in size.

### FIELD OF THE INVENTION

The present invention relates to a method for predicting and monitoring the severity of COVID-19 disease following infection of a subject with the SARS-CoV-2 virus.

There is no specific and objective clinical test to determine or predict COVID-19 disease severity. Currently, clinicians use several generic readouts (e.g. blood oxygen saturation, interleukin-6 concentration) and their clinical judgement. Unique to COVID-19, there is a problem around timelines where any required tests need to be designed, developed and deployed rapidly.

Clinicians use a variety of generic readouts to try to determine and monitor disease severity. One of the more widely spread measurements is blood oxygen saturation. Interleukin-6 concentration may also be used among other, more standard blood tests and clinical readouts. Prior to the making of the present invention, it is believed that there is no specific commercially available blood test for COVID-19 disease severity.

A recent paper by Messner et al (Cell Systems, vol. 11, pages 11-24 (2020)) describes the use of ultra-high-throughput clinical proteomics to reveal classifiers of COVID-19 infection. The report is focused on an early proteomics signature for COVID-19 disease classifiers and deals with the discovery stage of the protein signature. However, the report does not suggest any means to predict the severity of the COVID-19 disease in infected subjects.

The technique described by Messner et al is also an unbiased discovery mass spectrometry analysis used in the context of discovery only and not for prognosis of a patient's disease status. Another paper by Demichev et al (2020) reports a time-resolved proteomic and diagnostic assay for COVID-19 disease progression in which a broad group of protein markers is studied (doi.org/10.1101/2020.11.09.20228015).

The present invention provides a specific clinical test to classify and predict COVID-19 disease severity. The test uses a previously undisclosed combination of 31 proteins and 52 peptide sequences therefrom, where the peptide sequences relied on have not been previously disclosed and at least some of the proteins have not previously been associated with COVID-19 disease. The invention is a targeted proteomics blood test to predict and monitor COVID-19 disease severity. The blood test measures the absolute concentration of 52 peptides arising from 31 blood plasma proteins at the same time. The heavily multiplexed assay is definitive and can be accredited to existing regulatory standards to deploy in the clinic.

In accordance with a first aspect of the invention, there is provided a method for predicting and/or classifying the severity of COVID-19 disease in a subject, the method comprising:
(i) preparing a biological sample from the subject for assay by incubating the sample with a protease to form a proteolytic digest of proteins in the sample; and
(ii) assaying the proteolytic digest of proteins of step (i) for the presence of a proteolytic peptide of at least one protein selected from the group of proteins as shown in Table 1, said group consisting of Proteoglycan 4, Inter-alpha-trypsin inhibitor heavy chain H1, Plasminogen (EC 3.4.21.7), Actin (Actin, aortic smooth muscle; Actin, cytoplasmic 1; Actin, cytoplasmic 2; Actin, gamma-enteric smooth muscle), Complement C1q subcomponent subunit C, Cystatin-C, Protein ORM2, Alpha-1-antichymotrypsin, Serotransferrin, Apolipoprotein B-100, EGF-containing fibulin-like extracellular matrix protein 1, von Willebrand factor, C-reactive protein, Lysozyme C (EC 3.2.1.17), Apolipoprotein A-I, Alpha-2-HS-glycoprotein, Histidine-rich glycoprotein, Beta-2-microglobulin, N-acetylmuramoyl-L-alanine amidase (EC 3.5.1.28), Transthyretin, Plasma kallikrein (EC 3.4.21.34), Antithrombin-III, Heparin cofactor 2, Afamin, Plasma protease C1 inhibitor, Transferrin receptor protein 1, Low affinity immunoglobulin gamma Fc region receptor III-A, Monocyte differentiation antigen CD14, Insulin-like growth factor-binding protein complex acid labile subunit, Immunoglobulin heavy variable 5-51, or Complement C3,
wherein the presence of said proteolytic peptide is assayed for using mass spectrometry with reference to a corresponding labelled and/or unlabelled reference proteolytic peptide.

The protease may be a serine protease (e.g. trypsin, chymotrypsin, thrombin, elastase, or subtilisin), a cysteine protease (e.g. papain, caspase-1, adenain, pyroglutamly-peptidase I, sortase A, hepatitis C virus peptidase 2, sindbis virus-type nsP2 peptidase, dipeptidyl-peptidase VI, DeSI-1 peptidase, TEV protease, amidophosphoribozyltransferase precursor, gamma-glutamyl hydrolase, hedgehog protein, dmpA aminopeptidease), a threonine protease (e.g. ornithine acetyltransferase), an aspartic protease (e.g. pepsin, cathepsin D, cathepsin E, napsin-A, nepenthesin, presenilin, renin (chymosin)), a glutamic protease (e.g. scytalidoglutamic peptidase (eqolisin), aspergilloglutamic peptidase), a metalloprotease (e.g. an ADAM or a matrix metalloproteinase), or an asparagine peptide lyase.

A proteolytic peptide is therefore a peptide sequence from a protein which has been produced by the action of a protease cleaving a peptide bond between amino acids in the protein sequence. Such proteolytic peptides are therefore oligopeptides being formed of a number of amino acids. The proteolytic peptide may be from 5 to 30 amino acid residues in length, suitably 6 to 25 amino acids in length, 7 to 21 amino acids in length, or any of 5, 6, 7, 8, 9, 10, 11, 12, 13, 14, 15, 16, 17, 18, 19, 20, 21, 22, 23, 24, 25, 26, 27, 28, 29 or 30 amino acids in length.

A proteolytic peptide prepared by the action of the protease trypsin on a protein may be referred to as a tryptic peptide and so on.

The proteolytic peptide may suitably have a sequence as set out in Table 1.

The method of the invention may comprise assaying for the presence of up to all 31 proteins in Table 1 with respect to a proteolytic peptide thereof. In some embodiments, the method of the invention may comprise assaying for the presence of a proteolytic peptide of at least 5, 6, 7, 8, 9, 10, 11, 12, 13, 14, 15, 16, 17, 18, 19, 20, 21, 22, 23, 24, 25, 26, 27, 28, 29, 30, or 31 proteins as shown in Table 1.

The methods of the invention therefore comprise the assaying for up to 52 proteolytic peptides of the 31 proteins shown in Table 1. In some embodiments, the method of the invention may comprise assaying for at least 5 to 10, 5 to 15, 5 to 20, 5 to 25, 5 to 30, 5 to 35, 5 to 40, 5 to 45 or 5 to 50 proteolytic peptides of Table 1. Preferably the number of proteolytic peptides assayed for is at least 7, at least 17, at least 29 or at least 41 proteolytic peptides as shown in Table 1.

References to proteolytic peptides as shown in Table 1 may alternatively refer to proteolytic peptides as shown in Table 6 or Table 9. The proteolytic peptide may suitably have a sequence as set out in Table 9 Cohort 1 and/or Table 9 Cohort 2.

Table 9 contains adjusted p values for cohort 1 and cohort 2. The lower the adjusted p value the more important the peptide is for stratification and outcome prediction. The method of the invention may suitably comprise assaying for proteolytic peptides from Table 9 with relatively low adjusted p values. The method may comprise assaying for the proteolytic peptide having the lowest adjusted p value from Table 9. The method may comprise assaying for the 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 proteolytic peptides having the lowest adjusted p values from Table 9. The method may comprise assaying for at least the 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 proteolytic peptides having the lowest adjusted p values from Table 9. The method may comprise assaying for at least the 2, 5, 10, 15, 20, 25, 30, 35, 40, or 45 proteolytic peptides having the lowest adjusted p values from Table 9.

The proteolytic peptides from Table 9 may be from cohort 1 and/or cohort 2. Where the proteolytic peptides are from cohort 1 the method may comprise assaying for the proteolytic peptide having the lowest adjusted p value from Table 9 cohort 1. Where the proteolytic peptides are from cohort 2 the method may comprise assaying for the proteolytic peptide having the lowest adjusted p value from Table 9 cohort 2. Where the proteolytic peptides are from cohort 1 and cohort 2 the method may comprise assaying for the proteolytic peptide having the lowest adjusted p value from Table 9 cohort 1 and for the proteolytic peptide having the lowest adjusted p value from Table 9 cohort 2. The method may comprise assaying for at least the proteolytic peptide having the lowest adjusted p value from Table 9 cohort 1 and for at least the proteolytic peptide having the lowest adjusted p value from Table 9 cohort 2, optionally further comprising at least the 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 proteolytic peptides having the lowest adjusted p values from Table 9 cohort 1 and/or optionally further comprising at least the 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 proteolytic peptides having the lowest adjusted p values from Table 9 cohort 2. The skilled person will appreciate that the method when the method comprises assaying for a proteolytic peptide on the basis of a relatively low adjusted p value in both Table 9 cohort 1 and Table 9 cohort 2, the method does not involve assessing the proteolytic peptide twice; rather the presence of a relatively low adjusted p value in Table 9 cohort 1 and Table 9 cohort 2 is a pointer to the skilled person to assess the proteolytic peptide once.

The method may comprise assaying for one or more proteolytic peptides having an adjusted p value from Table 9 cohort 1 and/or Table 9 cohort 2 which is at or below a threshold adjusted p value. The threshold adjusted p value may be 0.05, 0.01, 0.001, 0.0001 or 0.00001, 1×10⁻⁶, 1×10⁻⁷, 1×10⁻³, 1×10⁻⁹, 1×10⁻¹⁰, 1×10⁻¹, 1×10⁻¹², 1×10⁻¹³ or 1×10⁻¹⁴. The threshold adjusted p value from Table 9 cohort 1 may be 0.05, 0.01, 0.001, 0.0001 or 0.00001. The threshold adjusted p value from Table 9 cohort 2 may be 0.05, 0.01, 0.001, 0.0001 or 0.00001, 1×10⁻⁶, 1×10⁻⁷, 1×10⁻³, 1×10⁻⁹, 1×10⁻¹⁰, 1×10⁻¹, 1×10⁻¹², 1×10⁻¹³ or 1×10⁻¹⁴.

The method may therefore comprise:
(ii) assaying the proteolytic digest of proteins of step (i) for the presence of one or more proteolytic peptides as shown in Table 9 Cohort 1, said group consisting of:

(SEQ ID No.: 11)
WEMPFDPQDTHQSR
(SEQ ID No.: 12)
EQLSLLDR
(SEQ ID No.: 27)
CNLLAEK
(SEQ ID No.: 21)
GYSIFSYATK
(SEQ ID No.: 26)
TVVQPSVGAAAGPVVPPCPGR
(SEQ ID No.: 13)
GDVAFVK
(SEQ ID No.: 14)
WCALSHHER
(SEQ ID No.: 25)
ATEHLSTLSEK
(SEQ ID No.: 24)
AHVDALR
(SEQ ID No.: 43)
LVLLNAIYLSAK
(SEQ ID No.: 8)
ALDFAVGEYNK
(SEQ ID No.: 10)
SDVMYTDWK
(SEQ ID No.: 42)
LLDSLPSDTR
(SEQ ID No.: 23)
YWCNDGK
(SEQ ID No.: 52)
VHQYFNVELIQPGAVK
(SEQ ID No.: 34)
GSPAINVAVHVFR
(SEQ ID No.: 1)
GLPNWTSAISLPNIR
(SEQ ID No.: 6)
FNAVLTNPQGDYDTSTGK
(SEQ ID No.: 33)
TFTLLDPK
(SEQ ID No.: 18)
ILTSDVFQDCNK
(SEQ ID No.: 49)
LAELPADALGPLQR
(SEQ ID No.: 16)
EQHLFLPFSYK
(SEQ ID No.: 17)
ADQVCINLR
(SEQ ID No.: 7)
TNQVNSGGVLLR
(SEQ ID No.: 35)
AADDTWEPFASGK
(SEQ ID No.: 32)
GCPDVQASLPDAK
(SEQ ID No.: 38)
ANRPFLVFIR
(SEQ ID No.: 2)
GHMLENHVER
(SEQ ID No.: 41)
TINPAVDHCCK
(SEQ ID No.: 40)
HFQNLGK
(SEQ ID No.: 37)
IAYGTQGSSGYSLR
(SEQ ID No.: 20)
ESDTSYVSLK
(SEQ ID No.: 15)
IAELSATAQEIIK
(SEQ ID No.: 22)
STDYGIFQINSR
(SEQ ID No.: 39)
TSCLLFMGR
(SEQ ID No.: 44)
ILNIFGVIK
(SEQ ID No.: 48)
DFALQNPSAVPR
(SEQ ID No.: 3)
EAQLPVIENK
(SEQ ID No.: 28)
GGEGTGYFVDFSVR
and
(SEQ ID No.: 4)
CQSWSSMTPHR.

The method may therefore comprise:
(ii) assaying the proteolytic digest of proteins of step (i) for the presence of one or more proteolytic peptides as shown in Table 9 Cohort 2, said group consisting of:

(SEQ ID No.: 33)
TFTLLDPK
(SEQ ID No.: 32)
GCPDVQASLPDAK
(SEQ ID No.: 20)
ESDTSYVSLK
(SEQ ID No.: 21)
GYSIFSYATK
(SEQ ID No.: 27)
CNLLAEK
(SEQ ID No.: 26)
TWQPSVGAAAGPWPPCPGR
(SEQ ID No.: 13)
GDVAFVK
(SEQ ID No.: 36)
DSVTGTLPK
(SEQ ID No.: 24)
AHVDALR
(SEQ ID No.: 25)
ATEHLSTLSEK
(SEQ ID No.: 14)
WCALSHHER
(SEQ ID No.: 49)
LAELPADALGPLQR
(SEQ ID No.: 48)
DFALQNPSAVPR
(SEQ ID No.: 41)
TINPAVDHCCK
(SEQ ID No.: 12)
EQLSLLDR
(SEQ ID No.: 40)
HFQNLGK
(SEQ ID No.: 2)
GHMLENHVER
(SEQ ID No.: 11)
WEMPFDPQDTHQSR
(SEQ ID No.: 5)
EITALAPSTMK
(SEQ ID No.: 8)
ALDFAVGEYNK
(SEQ ID No.: 47)
VLDLSCNR
(SEQ ID No.: 38)
ANRPFLVFIR
(SEQ ID No.: 29)
IADAHLDR
(SEQ ID No.: 37)
IAYGTQGSSGYSLR
(SEQ ID No.: 51)
VEGTAFVIFGIQDGEQR
(SEQ ID No.: 19)
YAGSQVASTSEVLK
(SEQ ID No.: 39)
TSCLLFMGR
(SEQ ID No.: 3)
EAQLPVIENK
(SEQ ID No.: 28)
GGEGTGYFVDFSVR
(SEQ ID No.: 15)
IAELSATAQEIIK
(SEQ ID No.: 17)
ADQVCINLR
(SEQ ID No.: 35)
AADDTWEPFASGK
(SEQ ID No.: 18)
ILTSDVFQDCNK
(SEQ ID No.: 34)
GSPAINVAVHVFR
(SEQ ID No.: 16)
EQHLFLPFSYK
(SEQ ID No.: 4)
CQSWSSMTPHR
(SEQ ID No.: 42)
LLDSLPSDTR
(SEQ ID No.: 23)
YWCNDGK
(SEQ ID No.: 43)
LVLLNAIYLSAK
(SEQ ID No.: 6)
FNAVLTNPQGDYDTSTGK
(SEQ ID No.: 22)
STDYGIFQINSR
(SEQ ID No.: 52)
VHQYFNVELIQPGAVK
(SEQ ID No.: 45)
VSASPLLYTLIEK
(SEQ ID No.: 44)
ILNIFGVIK
(SEQ ID No.: 10)
SDVMYTDWK
(SEQ ID No.: 1)
GLPNWTSAISLPNIR
(SEQ ID No.: 7)
TNQVNSGGVLLR
and
(SEQ ID No.: 46)
DSGSYFCR.

The method may comprise assaying for any combination of proteolytic peptides from Table 9 cohort 1 and/or Table 9 cohort 2.

The top-right panel of supplementary FIG. 4 shows the relative importance of peptides for outcome prediction (extra trees model). The method may comprise assaying for one or more proteolytic peptides shown in the top-right panel of supplementary FIG. 4. The method may comprise assaying for at least 2, at least 3, at least 4, at least 5, at least 6, at least 7, at least 8, at least 9, at least 10, at least 11, at least 12, at least 13, at least 14 or all 15 proteolytic peptides shown in the top-right panel of supplementary FIG. 4. Preferably the method comprises assaying for a proteolytic peptide of high relative importance. The method may therefore comprise assaying for GCPDVQASLPDAK (SEQ ID No: 32) of PGLYRP2, which has a comparatively high relative importance of 8.0% (see top-right panel of supplementary FIG. 4). The method may comprise assaying for one or more proteolytic peptides having a relative importance which is at or above a threshold relative importance. The threshold relative importance may be 2%, 2.5%, 3%, 3.5%, 4%, 4.5%, 5%, 6% or 7.5%.

The method may therefore comprise (ii) assaying the proteolytic digest of proteins of step (i) for the presence of one or more proteolytic peptides as shown in the top-right panel of Supplementary FIG. 4, said group consisting of:

(SEQ ID No.: 32)
GCPDVQASLPDAK
(SEQ ID No.: 20)
ESDTSYVSLK
(SEQ ID No.: 27)
CNLLAEK
(SEQ ID No.: 26)
TWQPSVGAAAGPWPPCPGR
(SEQ ID No.: 4)
CQSWSSMTPHR
(SEQ ID No.: 8)
ALDFAVGEYNK
(SEQ ID No.: 13)
GDVAFVK
(SEQ ID No.: 38)
ANRPFLVFIR
(SEQ ID No.: 21)
GYSIFSYATK
(SEQ ID No.: 3)
EAQLPVIENK
(SEQ ID No.: 14)
WCALSHHER
(SEQ ID No.: 24)
AHVDALR
(SEQ ID No.: 49)
LAELPADALGPLQR
(SEQ ID No.: 29)
IADAHLDR
(SEQ ID No.: 48)
DFALQNPSAVPR.

The method may comprise assaying for any combination of proteolytic peptides from the top-right panel of supplementary FIG. 4.

In one embodiment, the method of the invention may suitably comprise assaying for the seven proteolytic peptides in the following group of proteolytic peptides:

(SEQ ID No.: 16)
EQHLFLPFSYK
(SEQ ID No.: 47)
VLDLSCNR
(SEQ ID No.: 21)
GYSIFSYATK
(SEQ ID No.: 43)
LVLLNAIYLSAK
(SEQ ID No.: 29)
IADAHLDR
(SEQ ID No.: 33)
TFTLLDPK
(SEQ ID No.: 32)
GCPDVQASLPDAK

According to this aspect of the invention, one or more additional peptide sequences may also be assayed for from the peptides shown in Table 1 in addition to the peptide sequences shown above. Suitably, the peptide sequences assayed for may therefore be as shown in Figures, 1, 2, 3 or 4.

Suitably, the peptide sequences assayed for may be as shown in Figures, 7, 8 or 9.

Suitably, the peptide sequences assayed for may be as shown in Supplementary Figures, 1, 2, 3 or 4.

In one embodiment, the method of the invention may suitably comprise assaying for the 32 proteolytic peptides that changed with the severity of the COVID-19 disease according to treatment escalation: i.e. from uninfected (WHO 0) to mild (WHO3), moderate (WHO 4, 5) and severely (WHO 6, 7) COVID-19 affected individuals (FIG. 8a, Supplementary FIG. 1, P<0.05; Table 9—Cohort 1).

In one embodiment, the method of the invention may suitably comprise assaying for the 33 proteolytic peptides that had a significant trend between patients according to the WHO ordinal outcome scale for clinical improvement with patients capturing a WHO score from relatively mild (WHO 3) to very severe cases (WHO 7) (Supplementary FIG. 3; Table 9—Cohort 2).

The methods of the present invention use mass spectrometry to identify the concentrations of the peptides, for example proteolytic peptides having the sequences as set out in Table 1. In a mass spectrometry (MS) system for the analysis of proteolytic peptides in a sample, the proteolytic peptides are injected into the MS system where the proteolytic peptides are first detected as intact peptides and then subsequently fragmented into smaller pieces which may be termed peptide fragments. The methods of the present invention provide for the detection of up to 52 proteolytic peptides of the up to 31 proteins as shown in Table 1. The concentrations of the peptides are used to assign a subject to a given level of severity of COVID-19 disease with reference to a calibration curve of the concentrations for known reference proteolytic peptides. For example, one scale for determining the severity of COVID-19 disease in a patient is the WHO scale of COVID-19 disease severity. In one embodiment of the invention, the determination of the severity of COVID-19 disease may be made according to the WHO scale of COVID-19 disease severity. The scale of COVID-19 disease severity may therefore be of from 0 to 10 using the WHO scale. Other scaling systems may be used also based on the concentrations of the peptides assayed for in the sample with reference to a calibration curve based on known standard proteolytic peptides. The disease severity correlates to the absolute concentration of peptides in a sample from a subject. Suitably, the concentrations of the proteolytic peptides in a sample are used in a linear regression model to calculate a risk score for a patent to develop a severe disease, for example to assign a subject to a defined grade of COVID-19 severity according to the WHO scale. The overall risk score may therefore be generated using a statistical model taking proteolytic peptide concentration and patient age as the relevant inputs.

The methods of the invention may further optionally also include the use of a statistical model which incorporates the concentration of proteolytic peptides determined according to the mass spectrometry analysis with the patients age in order to determine an overall risk for COVID-19 disease severity.

The concentrations of the proteolytic peptides assayed for in the samples are therefore linked with the prediction and/or diagnosis of COVID-19 disease severity. Generally, the higher the dysregulation of proteolytic peptide concentration from a baseline control (either increased or decreased), the more severe the disease is or will be. The measurement of peptide concentration variability within a disease stage/group is also important for accurate prediction and/or diagnosis. The lower the variability the more accurate the prediction or diagnosis. The ANOVA p-value is therefore a proxy that takes both of these components into account and determines which peptides are critical for prediction and/or diagnosis.

In the data presented herein in Table 4, the peptide EQHLFLPFSYK (SEQ ID No: 16) has the lowest p-value and a clear concentration increase in WHO7 patients vs. healthy or WHO3-WHO4 patients (FIG. 1, top left corner graph). Similarly, the same can be observed for the peptide VLDLSCNR (SEQ ID No: 47).

Reference peptides may be used to pre-configure the mass spectrometer prior to use in a method of the invention to detect and quantitate the concentration of peptides of Table 1 in a sample. The reference peptides also allow for the construction of calibration lines with each batch of samples tested in order to ensure robust results. Heavy isotope-labelled peptides may be used as internal standards to control analytical variability in each sample and also provide for calibration lines.

The methods of the invention may further account for whether the proteolytic peptides are shown herein to be up-regulated or downregulated in patients. See for example the 11 up-regulated and 22 downregulated proteolytic peptides described in Example 3. See also panel a of FIG. 8. The predicting and/or classifying the severity of COVID-19 disease may account for whether said proteolytic peptides are shown herein to be up-regulated or downregulated.

Novel peptide sequences of proteins not previously associated with detection of COVID-19 disease include sequences from:
EGF-containing fibulin-like extracellular matrix protein 1
Low affinity immunoglobulin gamma Fc region receptor III-A

The WHO scale of COVID-19 disease severity lists the patient state and the clinical descriptor against a score as follows (Marshall et al., The Lancet, vol. 20, e192-e197 (2020)):

| Patient State | Descriptor | Score |
|---|---|---|
| Uninfected | Uninfected; no viral RNA detected | 0 |
| Ambulatory mild | Asymptomatic; viral RNA detected | 1 |
| disease | Symptomatic; independent | 2 |
| | Symptomatic; assistance needed | 3 |
| Hospitalised: moderate | Hospitalised; no oxygen therapy | 4 |
| disease | Hospitalised; oxygen by mask or | 5 |
| | nasal prongs | |
| Hospitalised: severe | Hospitalised; oxygen by Non-invasive | 6 |
| diseases | ventilation (NIV) or high-flow | |
| | Intubation and mechanical ventilation, | 7 |
| | pO₂/FiO₂ ≥150 or SpO₂/FiO₂ ≥200 | |
| | Mechanical ventilation pO₂/FiO₂ <150 | 8 |
| | (SpO₂/FiO₂ <200) or vasopressors | |
| | Mechanical ventilation pO₂/FiO₂ <150 and | 9 |
| | vasopressors, dialysis or extracorporeal | |
| | membrane oxygenation (ECMO) | |
| Dead | Death | 10 |

The methods of the present invention are able to stratify patients according to the above scale with respect to those patients requiring hospitalisation, i.e. having a WHO scale of COVID-19 disease severity score of 3 or above, compared to those patients who do not require hospitalisation, i.e. having a WHO scale of COVID-19 disease severity score of below 3. For any patient sample, therefore, the methods of the invention provide a means to differentiate between patients requiring hospitalisation and those who do not require such treatment.

The methods of the invention therefore provide for the classification and prediction of COVID-19 severity. Clearly, the predictive aspect of the methods of great value to patients and to health systems in terms of being able to prioritise utilisation of resources and assess the outcome of an infection.

Accordingly, the methods of the invention provide for the detection of a patient requiring therapy for the treatment of COVID-19. The patient may be symptomatic or asymptomatic with respect to infection by SARS-CoV-2 virus. The therapy may be for treatment of COVID-19 disease with a WHO severity score of 3 or above. The therapy may comprise a drug therapy, or oxygen therapy. The oxygen therapy may be non-invasive or invasive. The oxygen therapy may comprise mechanical ventilation of the patient.

The test of the invention may suitably be performed as a blood test. Such a test may be conducted by collection of venous blood or potentially by a finger-prick blood collection device or a bloodspot card or plasmaspot card. The method of the present invention may be suitably performed on a sample of blood plasma or serum. In one embodiment, the test is conducted on citrate plasma (e.g. plasma to which sodium citrate has been added), but different plasma sample additives (anticoagulants) may also be used, for example, K2 and K3 EDTA plasma tubes, heparin, potassium oxalate/sodium fluoride treated plasma tubes and others. Samples based on serum, whole blood (venous or peripheral) or bloodspot or plasma-spot samples, cerebrospinal fluid (CSF), interstitial fluid, lymph fluid, urine, faeces and/or tissue biopsies may also be used according to the invention.

Any of the tests described herein may be multiplexed with another test measuring proteins in a biological sample, e.g. blood plasma proteins, with the same technology platform. Thus, the test of the present invention may be a part of a larger test. If other disease severity tests emerge, the test of the present invention could be used to augment such other tests to enhance overall performance.

The test of the present invention not only provides for an assessment of the presence of COVID-19 disease but also allows for a prediction of the severity of disease. The test provides for prediction of the need for specific treatment options (i.e. mechanical ventilation). The test also predicts whether the patients are likely to survive or not if they have severe disease and does so on average 39 days before outcome.

As a sample type, plasma is also difficult to prepare for mass spectrometry applications. However, in the present invention samples based on plasma have been used successfully. The plasma sample preparation in the methods of the present invention avoids problems of the prior art (analytical signal suppression and variability) by selecting the optimal peptides for analysis with corresponding heavy isotope labelled-internal standards with digestion efficiency control tags and calibration curves. Without wishing to be bound by theory, plasma samples (specifically from venous blood) may be the most suitable form of sample for use in a method of the present invention. Other sources of plasma obtained from finger prick collection or bloodspot samples may be also be used.

The same plasma proteins may also be measured using a different technology platform. ELISA, SIMOA, Olink, Western Blot, or other immunoassay platforms may be used to the measure the same protein set. Aptamers (oligonucleotide or peptide molecules that bind to a specific target molecule) can be used instead of antibodies in similar assays.

The proteins from which the peptide sequences are derived are set out in Table 1 described herein. The method of the present invention may be a multiplexed assay. In contrast to earlier experimental uses of mass spectrometry in the study of COVID-19 disease in patients, the present invention suitably uses targeted mass spectrometry.

As set out herein, a different peptide set to that shown in Table 1 could also be measured from the same proteins as shown in Table 1 using the same targeted proteomics platform but using a different protease. The examples of the present invention described herein show one embodiment of a method of the invention, however different peptides from the same set of 31 proteins may be used. This includes peptides generated using the same protease as in this test (trypsin) or different proteases (LysC, GluC etc).

Typically, the mass spectrometry analysis of peptides according to a method of the present invention, is liquid chromatography-targeted mass spectrometry (LC-MS) using triple quadrupole instruments, operated in timed multiple reaction monitoring (MRM) mode.

A different mass spectrometry platform could be used to measure the same set of analytes. Whilst triple quadrupole mass spectrometry platforms, operated in timed MRM mode, are preferred for this test, there could be other mass spectrometry platforms or other data acquisition modes used to design around this test. One example is high resolution mass spectrometry platforms (e.g. Sciex 6600, Thermo Orbitrap or Waters QTOF-type instruments) where “pseudo” MRM or PRM modes could be used for measurements. Triple quadrupole instruments are robust and provide reproducible data so may be a preferred technology.

A wide variety of chromatography systems can be coupled to a mass spectrometry platform. The test of the present invention may be suitably used with “standard flow” (ml/min) liquid chromatography systems but lower flow systems may be used—in a range of μl/min or nl/min.

The method may comprise a targeted, LC-MRM or LC-SRM assay to be used on a conventional triple-quadrupole mass spectrometer. The mass spectrometer may be running routine-typical chromatography. The flow-rate may be about 800 μL*min-1.

Various peptide ionisation interface methods can be used prior to mass spectrometry analysis according to a method of the invention. The present invention may suitably use electrospray ionisation (ESI). However, the peptides can also be ionised using matrix-assisted laser desorption/ionization (MALDI) or desorption electrospray ionization (DESI) or atmospheric-pressure chemical ionization (APCI) or other ionisation methods.

The methods of the present invention therefore comprise the use of targeted proteomics. The technique comprises the quantification of specific, pre-selected proteins or proteolytic peptides from a given sample and requires a pre-existing understanding of disease biology to guide protein selection. The technique is therefore distinct from discovery proteomics which seeks to gather information about all proteins and proteolytic peptides in a sample without pre-existing knowledge/hypotheses around disease biology. Internal standards or calibration lines for every protein or peptide of interest are not and cannot be used in discovery proteomics. Discovery proteomics is also conducted using different instrument operation modes, e.g. SWATH, HDMSE etc comparted to MRM or PRM in targeted proteomics. Data processing also uses different approach to signal normalisation and quantification where absolute concentration cannot be provided. Discovery proteomics platforms lack robustness and reproducibility of targeted proteomics platforms. Targeted and discovery proteomics are distinct to the extent that a team of scientists utilising discovery proteomics platforms are generally not able to develop a targeted proteomics biomarker test without specific knowledge and experience in targeted proteomics. This is due to the above mentioned and other differences at every stage of the process, from initial concepts, sample preparation, data acquisition and processing to final test implementation in a clinical setting.

The methods of the invention are performed using a corresponding labelled reference peptide or labelled reference proteolytic peptides and may be configured to use any suitable internal standard on the same targeted proteomics platform. The methods of the invention may suitably be used with heavy isotope-labelled internal standards for accurate measurements. Examples of internal standards as heavy-isotope labelled proteolytic peptide are shown in Table 1 with respect to the proteolytic peptides described therein. The internal standard may be added to the sample before a proteolytic digestion of the proteins in the sample has occurred in step (i) of the methods of the invention. Alternatively, internal standard may be added to the sample after a proteolytic digestion of the proteins in the sample has occurred in step (i) of the methods of the invention. The mass spectrometer may be pre-configured using said internal standard. Suitable, heavy-isotope labels are ¹³C, ¹⁵N; and/or ²H.

The method may comprise predicting and/or classifying the severity of COVID-19 disease in a subject using a trained machine learning model. Such a machine learning model may be used to predict and/or classify the severity of COVID-19 disease in a subject based on data such as one or more of: proteolytic peptide profiles (including individual concentrations for each proteolytic peptide for each patient); clinical scores such as CCI, SOFA, APACHE II and ABCS; patient state and/or descriptor; patient WHO grade; and specific levels of at least one proteolytic peptide. The data may be separated based on the specific WHO scale of the patient.

The machine learning model may be created using various scripts, such as Python or R scripts, to create Support Vector Machine (SVM) models, for example. The machine learning model may therefore comprise an SVM model. The skilled person is familiar with SVM models, and as such a specific implementation of SVM models will now be briefly described.

The SVM models may be created using the known and freely-accessible package ClassyFire developed by the Wishart Research Group (http://classyfire.wishartlab.com/), and described in the publication: Djoumbou Feunang Y, Eisner R, Knox C, Chepelev L, Hastings J, Owen G, Fahy E, Steinbeck C, Subramanian S, Bolton E, Greiner R, and Wishart D S. ClassyFire: Automated Chemical Classification With A Comprehensive, Computable Taxonomy. Journal of Cheminformatics, 2016, 8:61. As the skilled person would understand, ClassyFire is a web-based application for automated structural classification of chemical entities. ClassyFire uses an SVM to create the machine learning models. As would be understood, SVM classifies, makes a regression, and creates a novelty detection for the creation of the model. Several such models may be created until the most accurate model is found. Validation of the models is achieved using a validation cohort to estimate the Matthews Correlation Coefficient (MCC) value and assess the accuracy of the prediction, as would be understood. These SVM models output accuracy percentages and MCC values after validation.

The SVM models are trained based on training data. Such data includes “explanatory” data and “response” data for patients in which the severity of COVID-19 disease is already knows. The explanatory data comprises all the data that is used to determine the severity of COVID-19 disease in a subject. For example, the explanatory data may be the levels of at least one proteolytic peptide for a particular subject, including the individual concentrations of proteolytic peptides as obtained from the sample. The response data comprises data indicating the actual severity of COVID-19 disease in a subject. The training data may therefore comprise a tab-delimited table with a training dataset of subjects as columns and proteolytic peptide levels as rows, and a tab-delimited table with a validation dataset of subjects as columns and proteolytic peptide levels as rows.

The method may therefore comprise measuring the levels of at least one proteolytic peptide and predicting and/or classifying the severity of COVID-19 disease, the method comprising inputting the levels of at least one proteolytic peptide into a trained machine learning algorithm, the trained machine learning algorithm being arranged to:
i. Compare the level of the at least one proteolytic peptide from the sample to a baseline control, and
ii. Output a prediction and/or classification of the severity of COVID-19 disease in a subject;

optionally wherein the trained machine learning model is a Support Vector Machine (SVM) model.

The trained machine learning algorithm may be trained based on training data, the training data comprising:
First data including proteolytic peptide levels for a plurality of subjects; and
Second data identifying the severity of COVID-19 disease for each of the plurality of subjects.

The methods of the present invention may comprise steps performed by a computer and involve equipment controlled by the computer. The step of assaying the proteolytic digest of proteins of step (i) for the presence of a proteolytic peptide may be performed by equipment controlled by the computer.

The invention also provides a computer-implemented method predicting and/or classifying the severity of COVID-19 disease in a subject, which comprises receiving in a computer sample data representing the level of at least one proteolytic peptide in sample obtained from a subject and executing software on the computer to compare the level of the at least at least one proteolytic peptide in the sample to a baseline control, wherein the difference between the level of the at least one proteolytic peptide and the baseline control is indicative of the severity of COVID-19 disease in the subject, and to output severity data representing the severity of COVID-19 disease in the subject on the basis of the comparison.

The invention also provides a computer program comprising instructions which, when executed by a computer, cause the computer to carry out a computer implemented method of the invention.

It will be appreciated that the step of comparing the level of the at least at least one proteolytic peptide in the sample with a baseline control may be carried out on a different computer from a computer that initially receives data representing the at least at least one proteolytic peptide in the sample.

The invention also provides a computer apparatus for assessing the severity of COVID-19 disease in a subject, which comprises a first device incorporating a computer, a second computer and a communication channel between the first device and second computer for the transmission of data therebetween; wherein the first device is arranged to receive sample data representing level of the at least one proteolytic peptide in a sample obtained from the subject and to transmit the sample data to the second computer via the communication channel, and the second computer is arranged to execute software to compare levels of the at least at least one proteolytic peptide in the sample to a baseline control to determine the severity of COVID-19 disease in the subject, wherein the difference between the level of the at least one proteolytic peptide and the baseline control is indicative of the severity of COVID-19 disease in the subject, and to output severity data representing the severity of COVID-19 disease in the subject on the basis of the comparison.

The second computer may be arranged to transmit the severity data to the first device via the communication channel, or to a third computer.

In some embodiments, the first device may incorporate mass spectrometry equipment or devices for measuring the level of at least one proteolytic peptide in a sample.

In accordance with a second aspect of the invention, there is provided a method for the treatment of a subject with COVID-19 disease, the method comprising:
(i) preparing a biological sample from the subject for assay by incubating the sample with a protease to form a proteolytic digest of proteins in the sample;
(ii) assaying the proteolytic digest of proteins of step (i) for the presence of a proteolytic peptide of at least one protein selected from the group of proteins as shown in Table 1, said group consisting of Proteoglycan 4, Inter-alpha-trypsin inhibitor heavy chain H1, Plasminogen (EC 3.4.21.7), Actin (Actin, aortic smooth muscle; Actin, cytoplasmic 1; Actin, cytoplasmic 2; Actin, gamma-enteric smooth muscle), Complement C1q subcomponent subunit C, Cystatin-C, Protein ORM2, Alpha-1-antichymotrypsin, Serotransferrin, Apolipoprotein B-100, EGF-containing fibulin-like extracellular matrix protein 1, von Willebrand factor, C-reactive protein, Lysozyme C (EC 3.2.1.17), Apolipoprotein A-I, Alpha-2-HS-glycoprotein, Histidine-rich glycoprotein, Beta-2-microglobulin, N-acetylmuramoyl-L-alanine amidase (EC 3.5.1.28), Transthyretin, Plasma kallikrein (EC 3.4.21.34), Antithrombin-III, Heparin cofactor 2, Afamin, Plasma protease C1 inhibitor, Transferrin receptor protein 1, Low affinity immunoglobulin gamma Fc region receptor III-A, Monocyte differentiation antigen CD14, Insulin-like growth factor-binding protein complex acid labile subunit, Immunoglobulin heavy variable 5-51, or Complement C3,
wherein the presence of said proteolytic peptide is assayed for using mass spectrometry with reference to a corresponding labelled and/or unlabelled reference proteolytic peptide; and
(iii) treating the subject with a therapeutic agent or treatment according to the severity of the COVID-19 disease detected in the subject.

Details of the methods of the second aspect of the invention are as for the first aspect as described above.

Suitable treatment of a subject suffering from COVID-19 disease will depend on the severity of the disease state of the subject (i.e. the patient). Treatment may comprise oxygen therapy, supply of oxygen by non-invasive ventilation (NIV) or high flow, intubation and mechanical ventilation, pO₂/FiO₂≥150 or SpO₂/FiO₂≥200, mechanical ventilation pO₂/FiO₂<150 (SpO₂/FiO₂<200) or vasopressors, or mechanical ventilation pO₂/FiO₂<150 and vasopressors, dialysis or extracorporeal membrane oxygenation (ECMO).

Patients at all levels of severity of disease may be treated with appropriate therapeutic agents. Suitable therapeutic agents may comprise, but are not limited to steroids, non-steroidal anti-inflammatory agents (NSAIDs) and/or anti-viral agents, and/or derivatives or salts thereof, antibodies, donor plasma, cells and/or products of cells.

Examples of suitable steroids are corticosteroids, such as dexamethasone and/or derivatives or salts thereof (e.g. dexamethasone sodium phosphate).

Examples of suitable non-steroidal anti-inflammatory agents (NSAIDs) are paracetamol (acetaminophen), ibuprofen, diclofenac and/or ketorolac, and/or salts or derivatives thereof.

Examples of suitable anti-viral agents are remdesivir and/or derivatives thereof.

Examples of suitable antibodies are tocilizumab, bamlanivimab, casirivimab, and imdevimab, or combinations thereof.

Examples of cell-based medicines or products thereof are mesenchymal stem cells (e.g. remestemcel-L) and the secretome of cultured amnion-derived epithelial cells (e.g. ST266).

The therapeutically active substance may be administered alone or in combination. Where therapeutically active substances are administered in combination, the administration may separate, simultaneous or subsequent.

If the patient is characterized as being “Hospitalized Mild Disease” at WHO Score Level 4, then suitably therapy may comprise oxygenation, e.g. oxygen delivered by mask or nasal prongs.

If the patient is characterized as being “Hospitalised Severe Disease” at WHO Score Level 5, then suitable treatment may comprise non-invasive ventilation or high-flow oxygen, which may be supplemented by intubation and mechanical ventilation for patients at WHO Score Level 6.

If the patient is characterised characterized as being “Hospitalised Severe Disease” at WHO Score Level 7, then suitable treatment may comprise ventilation and additional organ support, administration of vasopressors, renal replacement therapy (RRT) and extracorporeal membrane oxygenation (ECMO).

According to a third aspect of the present invention, there is provided a kit for predicting and/or classifying the severity of COVID-19 disease in a subject according to a method of the first aspect of the present invention, comprising:
a plurality of sample preparation media for analysis of a sample by mass spectrometry for the presence of a proteolytic peptide of at least one protein selected from the group of proteins as shown in Table 1, said group consisting of Proteoglycan 4, Inter-alpha-trypsin inhibitor heavy chain H1, Plasminogen (EC 3.4.21.7), Actin (Actin, aortic smooth muscle; Actin, cytoplasmic 1; Actin, cytoplasmic 2; Actin, gamma-enteric smooth muscle), Complement C1q subcomponent subunit C, Cystatin-C, Protein ORM2, Alpha-1-antichymotrypsin, Serotransferrin, Apolipoprotein B-100, EGF-containing fibulin-like extracellular matrix protein 1, von Willebrand factor, C-reactive protein, Lysozyme C (EC 3.2.1.17), Apolipoprotein A-I, Alpha-2-HS-glycoprotein, Histidine-rich glycoprotein, Beta-2-microglobulin, N-acetylmuramoyl-L-alanine amidase (EC 3.5.1.28), Transthyretin, Plasma kallikrein (EC 3.4.21.34), Antithrombin-III, Heparin cofactor 2, Afamin, Plasma protease C1 inhibitor, Transferrin receptor protein 1, Low affinity immunoglobulin gamma Fc region receptor III-A, Monocyte differentiation antigen CD14, Insulin-like growth factor-binding protein complex acid labile subunit, Immunoglobulin heavy variable 5-51, or Complement C3.

Optionally, the kit may additionally comprise a biological collection device. The kit may also comprise instructions for use.

Suitable media may comprise one or more of the following selected from the group consisting of a peptide storage solvent (for example comprising aqueous acetonitrile, optionally comprising 50% acetonitrile/50% ddH₂O), a denaturation buffer (for example comprising urea, ammonium bicarbonate and dithiothreitol (DTT), optionally comprising 8M Urea, 100 mM ammonium bicarbonate, 50 mM DTT); an alkylation agent (for example comprising 2-iodoacetamide (IAA), optionally comprising 100 mM IAA); salts (for example comprising ammonium bicarbonate, optionally comprising 100 mM ammonium bicarbonate), and/or a surrogate matrix (for example bovine serum albumin (BSA), optionally comprising 40 mg/ml bovine serum albumin in ddH₂O).

Such a kit may include materials for use in mass spectrometry analysis of protein samples, e.g. protease(s), diluents and/or other media, optionally further comprising instructions for use of the kit in a method of the invention. The sample collection device may comprise additional preservatives and/or stabilisers and/or reference peptide standards, i.e. synthetic natural peptides or internal standards, such as heavy-isotope labelled peptides as described herein.

The kits of the invention may also be used to assess the efficacy of new therapeutics or vaccines in clinical or pre-clinical studies for the treatment of COVID-19 disease.

According to a fourth aspect of the invention, there is provided a pharmaceutical composition comprising a therapeutic agent for use in a method of treatment of a subject with COVID-19 disease, wherein the COVID-19 disease of the subject has been classified according to a method of the first aspect of the invention.

According to a fifth aspect of the invention, there is provided a kit for use in the treatment of a subject with COVID-19 disease according to a method according to the second aspect of the invention, comprising:
(i) a plurality of sample preparation media for analysis of a sample by mass spectrometry for the presence of a proteolytic peptide of at least one protein selected from the group of proteins as shown in Table 1, said group consisting of Proteoglycan 4, Inter-alpha-trypsin inhibitor heavy chain H1, Plasminogen (EC 3.4.21.7), Actin (Actin, aortic smooth muscle; Actin, cytoplasmic 1; Actin, cytoplasmic 2; Actin, gamma-enteric smooth muscle), Complement C1q subcomponent subunit C, Cystatin-C, Protein ORM2, Alpha-1-antichymotrypsin, Serotransferrin, Apolipoprotein B-100, EGF-containing fibulin-like extracellular matrix protein 1, von Willebrand factor, C-reactive protein, Lysozyme C (EC 3.2.1.17), Apolipoprotein A-I, Alpha-2-HS-glycoprotein, Histidine-rich glycoprotein, Beta-2-microglobulin, N-acetylmuramoyl-L-alanine amidase (EC 3.5.1.28), Transthyretin, Plasma kallikrein (EC 3.4.21.34), Antithrombin-III, Heparin cofactor 2, Afamin, Plasma protease C1 inhibitor, Transferrin receptor protein 1, Low affinity immunoglobulin gamma Fc region receptor III-A, Monocyte differentiation antigen CD14, Insulin-like growth factor-binding protein complex acid labile subunit, Immunoglobulin heavy variable 5-51, or Complement C3; and
(ii) a therapeutic agent for treatment of COVID-19 disease.

The test of the present invention advantageously provides an accurate measurement of COVID-19 disease severity. The test is set up on a robust, targeted mass spectrometry platform and uses synthetic reference and internal standards. The test allows rapid iteration and implementation of predictive protein signatures (e.g. for different patient groups/settings). The sample preparation method of the test is short, simple and can be manual or automated. The protein signature, which this test is based on, was discovered in thousands of proteomes from hundreds of patients with longitudinal follow up and across 3 cohorts which makes it particularly robust.

The present invention provides a test to assess and/or predict COVID-19 disease severity when patients arrive at hospital, including assessing and/or predicting COVID-19 disease severity throughout the time that the patient is being treated at hospital for COVID-19. The test can be used to plan bed occupancy and broader facilities, personnel and equipment use at hospital, including mechanical ventilator and supplementary oxygen use.

The test of the invention can be used to assess the effectiveness of current and future treatment regiments in individual patients at hospital or in primary care. The test can also be used to assess and/or predict COVID-19 disease severity when patients present at a GP surgery (in primary care) in order to direct patients to or away from hospitals.

The present invention provides a test to assess the efficacy of a COVID-19 therapeutic agent and/or vaccine in pre-clinical studies and/or clinical trials and/or post marketing authorisation pharmacovigilence. The COVID-19 therapeutic agent and/or vaccine can be newly developed or re-purposed to treat COVID-19, including convalescent plasma treatment and also different formulations and administration routes of the same therapeutic agent and/or vaccine.

The present invention provides a test to compare properties (e.g. virulence and resulting disease severity) of different or mutated strains (i.e. variants) of SARS-COV-2 in pre-clinical, animal and human studies, including viral strains known to infect animals and/or humans. The test can be used as a reference method/benchmark for future disease severity tests, which includes cross-validation at test development stages. The test can be used for population/epidemiological studies to determine which sub-groups of the population develop milder or more severe COVID-19, including differences in pre-existing vaccination status in different populations, specifically patients who have previously received a dose of a BCG tuberculosis (TB) vaccine or newly developed COVID-19 vaccines.

Preferred features of the second and subsequent aspects of the invention are as for the first aspect mutatis mutandis.

In one embodiment of the invention, the method of the first aspect may comprise
sample preparation, for example plasma obtained from a patient blood sample using standard protocols
protease digestion of proteins in sample prior to mass spectrometry analysis, for example by digesting proteins with trypsin to release tryptic peptides from proteins which can then be analysed using mass spectrometry, for example on a targeted LC-MS/MS platform.
mass spectrometry analysis is performed on the prepared samples by measuring proteolytic tryptic peptides, for example using a LC-MS/MS platform, suitably a pre-configured LC-MS/MS system which has been “programmed” to detect and quantify a specific set of peptides using corresponding internal standards, for example assaying for the proteolytic peptides as shown in Table 1 using the corresponding internal standards to prepare a calibration curve
determination of the proteolytic peptide concentration (suitably at μg/ml or ng/ml) is determined for all analyte peptides in the sample, for example tryptic peptide concentration, optionally up to all 52 proteolytic peptides as analytes
generation of overall severity score for patient using peptide concentration according to WHO score of COVID-19 severity and optionally also using patient's age in a statistical model to generate an overall risk score for each individual patient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Reference is made to the following drawings in which:

FIG. 1 shows results for 17 peptides used in the targeted LC-MS/MS assay for COVID-19 disease severity classification and prediction.

FIG. 2 shows results for 7 peptides used in the targeted LC-MS/MS assay for COVID-19 disease severity classification and prediction.

FIG. 3 shows results for 29 peptides used in the targeted LC-MS/MS assay for COVID-19 disease severity classification and prediction.

FIG. 4 shows results for 41 peptides used in the targeted LC-MS/MS assay for COVID-19 disease severity classification and prediction.

FIG. 5 shows results for 7 peptides used in the targeted LC-MS/MS assay for COVID-19 disease severity classification and prediction in which results are shown as a graph (Principal Component Analysis) to show how well different disease groups are separated when the test is used as a classifier of COVID-19 disease severity based on the 7 peptides referred to in FIG. 2.

FIG. 6 shows a schematic overview of the steps from peptide selection, to development and application of a COVID19 biomarker panel for testing disease severity and predicting outcome. We selected 50 proteotypic peptides derived from 30 plasma proteins as measured by discovery proteomics in a research setting (Demichev et al. 2021; Messner et al. 2020; Demichev et al. 2021), with the condition that they i) are predictive about the future time in hospital in a COVID19 patients, ii) are differentially expressed according to the treatment escalation determined by the WHO ordinal scale, and iii) are belonging to biological processes that are causally implicated in the COVID19 pathogenesis (including inflammatory response, complement cascade, metabolism, and the coagulation system) (top panel). To generate an assay that is suitable for clinical and routine laboratories, we established a targeted, LC-SRM assay to be used on conventional triple-quadrupole mass spectrometers, running routine-typical chromatography, at a flow-rate of 800 μL*min-1. The assay was optimized using synthetic peptides, and to achieve absolute quantification, we synthesized for each peptide an internal, heavy isotope labelled standard (‘AQUA peptide (Gerber et al. 2003)) with a short tryptic tag, to evaluate the sample preparation (tryptic digest) procedure. (middle panel). Then, the assay was analytically validated in two laboratories on two instruments. The power to predict disease severity and outcome was validated on an independent, well balanced second (‘1st wave’) COVID19 cohort whose samples were measured in two laboratories, and a larger (‘3rd wave’) cohort, treated at the Charite' Hospital which is a national biomedical reference center.

FIG. 7 A-C shows results for a biological and analytical validation of biomarker panel selection. a) Correlation of the abundance of the proteins chosen for the panel assay. The selected proteins i) participate in biological processes related to the COVID19 pathology, ii) are predictive about future worsening of COVID19 disease parameters over time (Demichev et al. 2021), iii) change in a concentration dependent manner depending on the treatment escalation level according the WHO ordinal scale, (Demichev et al. 2021; Messner et al. 2021; Messner et al. 2020), and their predictive value for the remaining time in hospital, used as a measure of disease severity (Demichev et al. 2021). Colored tiles indicate significant associations, with red/blue highlighting that the respective protein is up- or down-regulated in COVID19 infected individuals. b) Selected protein biomarkers and the COVID19-pathology biological process they are associated with (literature curation) c) Extracted ion chromatograms highlighting the chromatographic spread of the applied SRM transitions selected as quantifiers for the indicated proteins. Note that for a majority of proteins, we included two proteotypic peptides in our assay (colored as blue, black for illustration purposes).

FIG. 8 A-E shows results for a diagnostic and analytical performance of routine applicable LC-SRM based, COVID19 protein panel assay. a) The established assay was first applied on citrate plasma samples, collected for a balanced COVID19 cohort studied during the first wave of the pandemic (Messner et al. 2021) (REF PA COVID19) (‘Cohort 1’) consisting of healthy volunteers (n=15, WHO grade 0), mildly COVID19 affected individuals requiring hospitalisation but no oxygen therapy (n=10 (WHO 3), COVID19 affected individuals requiring hospitalisation and non-invasive oxygen therapy (n=4, WHO 4; n=3 WHO5), and severely affected hospitalized individuals requiring mechanical ventilation (n=3 (WHO 6), n=10 (WHO7) as well as QC plasma samples (n=12). Peptides with a significant concentration change (downregulated (top) and upregulated (bottom)) distinguish healthy from infected individuals, as well as mild from severe forms of the disease. Heatmap displays log 2 fold change of the indicated peptide to its median concentration in patients with a severity score of WHO3. To facilitate visualisation, peptides with log 2 fold-changes <−1.25 or >1.25 are indicated by the same respective color. Peptides are ordered based on significance, with most significant peptides of each respective panel on top. Only peptides with P<0.05 based on Kendall's Tau trend estimator after multiple testing corrections are shown. For additional information, see Supplementary FIG. 1. b) Representative peptide biomarkers indicating different COVID19 severity trends (changing with severity, differentiating healthy and mild disease, differentiating mild from severe disease, respectively). Boxplots display the absolute concentration of selected peptides in patients in different severity groups as explained in (a). Right panel: Selected SRM chromatoms from healthy (WHOO), mild (WHO3), and severely affected (WHO7) individuals, visualize the response to COVID19 on the selected biomarkers. c) Unsupervised clustering (shown is a principal component analysis (PCA)) based on the absolute quantification of all 39 quantified peptides (ADQVCINLR (SEQ ID No: 17) contained missing values and was omitted), stratifies (cohort 1) COVID19 patients by severity. d) Reproducibility of the protein panel assay in two laboratories, running two different LC-MS/MS platforms. Shown are linear correlations; Color code like in (c).

FIG. 9 A-E shows results for a diagnostic and analytical performance of the COVID19 panel assay on a COVID19 inpatient cohort treated during the 3rd wave of the pandemic. a) Quantitative performance (signal stability), during the measurement of 740 plasma proteome samples representing an inpatient cohort 3 of wave 3. n=85 QC samples (colored in grey, pool of COVID-19 samples as described in (Messner et al. 2020)) injected throughout the acquisition of cohort 2 (n=655 patient samples). Shown are the log 2 fold-change of the response ratios (peak area of naturally labelled over heavy labelled for each of the 48 quantified peptides) normalized to the median of the QC samples for the respective peptide. b) Peptide log 2 response-ratios of one selected up- (CST3) and down-regulated (PGLYRP2) protein for all samples acquired for cohort 2. QC samples (as described in a) are shown in grey, all other samples are colored according to the corresponding COVID19 WHO treatment escalation score; rug plots on the right side of each peptide indicate the distributions. c) Illustration of the results. The 16 most significantly (eight up- (top panel), and eight down-regulated (bottom panel)) peptide quantities that indicate COVID19 disease severity, expressed as the treatment level according to the WHO ordinal scale. One peptide per protein was selected; and one outlier (EITALAPSMK) was removed for visualisation purposes only. The quantities of all other peptides are illustrated in Supplementary FIG. 3. d) Principal Component analysis (PCA) of the absolute concentration of all quantified peptides; color-scheme according to (b). e. Confusion-matrix-like representation of the outcome of a multi-class classification model (SVM based) trained to differentiate three WHO severity groups: grade 3, grades 4/5, and grades 6/7. Predictions were done on withheld samples that were not used for training the models (accuracy=0.655, balanced accuracy=0.637). The position of the points within each square were chosen randomly. Color scheme according to (b).

FIG. 10 shows results for COVID-19 out prediction using a routine applicable LC-SRM assay. Receiver operator (ROC-Curve (left) for the prediction of survival and non-survival, from a single plasma sample collected the first time point measured for every patient using an SVM classifier. The blue curve denotes the model trained and benchmarked on measured proteomic data. The other curves denote models based on single severity scores (Sepsis-related Organ Failure Assessment score (SOFA, purple), Acute Physiology And Chronic Health Evaluation (APACHE II, green) and Charlsson Comorbidity Index (CCI, cyan)). Boxplot (center) of the decision function of the SVM sorted according to the outcome and colored with respect to the WHO grade at the day the sample was taken. Kaplan-Meier estimate of the survival function (right) for positive predicted cases (black) and negative predicted cases (orange) with confidence interval (alpha=0.05). All predictions were done on withheld samples that were not used for training the models.

FIG. 11 shows the abundance of selected peptides in different COVID-19 severity groups and healthy control samples. Absolute concentration of all peptides quantified in cohort 1 (samples obtained during the first wave of the pandemics) plotted against the COVID19 treatment escalation score. Note that the cohort also includes COVID-19 negative control samples (blue; WHO score=0). Peptides with a significant trend against COVID-19 severity as estimated on the respective WHO severity groups not-infected (0), mild (3), moderate (4 & 5) and severe (6 & 7) disease are highlighted in red (Kendall's Tau statistics, P>0.05 after multiple testing correction). Data from n=15 (WHO 0), n=10 (WHO 3), n=4 (WHO 4), n=3 (WHO 5), n=3 (WHO 6), n=10 (WHO7).

FIG. 12 shows a comparison of the obtained absolute quantities of the putative peptide biomarkers in plasma samples measured on two LC-QQQ platforms. Samples were prepared, split in two aliquots, and measured on two different LC-QQQ platforms, applying two independently optimized MRM transitions, and operated in two laboratories. Shown are the linear correlations of the absolute concentration of all quantified peptides. Obtained R2 values of the linear fit as indicated. Values on the y-axis (platform 1) were measured on a 6495C (Agilent) QQQ, values on y-axis (platform 2) were measured on a 7500 (Sciex) QQQ. All data based on n=15 (WHO 0), n=10 (WHO 3), n=4 (WHO 4), n=3 (WHO 5), n=3 (WHO 6), n=10 (WHO7) and n=12 (QC Plasma). Peptides are ordered alphabetically according to gene names. Peptides with poor correlation (R2<0.6) between both platforms are FNAVLTNPQGDYDTSTGK (SEQ ID No: 6) (C1QC), ESDTSYVSLK (SEQ ID No: 20) (CRP), DFALQNPSAVPR (SEQ ID No: 48) (IGFALS), STDYGIFQINSR (SEQ ID No: 22) (LYZ), GCPDVQASLPDAK (SEQ ID No: 32) (PGLYRP2), ILTSDVFQDCNK (SEQ ID No: 18) (VWF), ILNIFGVIK (SEQ ID No: 44) (TFRC). In the case of ESDTSYVSLK (SEQ ID No: 20), this is because of one outlier (removal increases the R2 to 0.76).

FIG. 13 shows the abundance of selected peptides in different COVID-19 severity groups. Absolute concentration of all peptides quantified in cohort 2 (plasma samples obtained during the second wave of the pandemic) plotted against the COVID-19 treatment escalation score. Peptides with a significant trend against COVID-19 severity as estimated on the respective WHO severity score are highlighted in red (Kendall's Tau statistics, P>0.05 after multiple testing correction). Data from the first time-point obtained for each individual; n=38 (WHO 3), n=45 (WHO 4), n=27 (WHO 5), n=16 (WHO 6), n=39 (WHO7). Peptides are ordered by their corresponding gene names.

FIG. 14 shows COVID19 progression prediction in a second wave COVID19 cohort using an ExtraTrees model. ROC-Curve (upper left) for the prediction of death from the first time point measured for every patient using an extra-trees classifier. Boxplot (lower left) of the predicted probability sorted according to the outcome and colored with respect to the WHO grade at the day the sample was taken. Kaplan-Meier estimate of the survival function (lower right) for positive predicted cases (black) and negative predicted cases (orange) with confidence interval (alpha=0.05). All predictions were done on withheld samples that were not used for training. Feature importances (upper right) for a model trained on all samples highlighting the corresponding protein/gene for each peptide.

Global healthcare systems continue to be challenged by the COVID-19 pandemic, and there is a need for clinical assays that can help to optimise resource allocation, support treatment decisions and accelerate the development and evaluation of new therapies. We developed a multiplexed proteomics assay and performed a stratification and prediction study on 2 cohorts of patients with COVID-19. The assay quantifies 50 peptides, derived from 30 known and newly introduced COVID-19 related protein markers, in a single measurement using analytical flow rate liquid chromatography and multiple reaction monitoring (LC-MRM), on equipment that is broadly available in routine laboratories. Technical analytical validation of the targeted, mass spectrometry-based peptide panel showed that it enables reproducible (i.e. inter-batch CV of 10.9%) absolute quantification of 47 peptides with high sensitivity (i.e. median LLOQ of 1.6 ng/ml) and accuracy (median 97.3%). Applied to two COVID-19 inpatient cohorts treated before and after dexamethasone became standard of care, composed of 30 and 164 patients, respectively, the assay reproducibly captured hallmarks of COVID-19 infection and severity, as it distinguished healthy subjects, mild, moderate and severe COVID-19. In the post dexamethasone cohort, the assay predicted survival with an accuracy of 0.83 (108/130), and death with an accuracy of 0.76 (26/34) in the median 2.5 weeks before the outcome, thereby outperforming compound clinical risk assessments such as SOFA, APACHE II, and ABCS scores. Disease severity and clinical outcomes of COVID-19 patients can be well stratified and predicted by this scalable and standardised protein panel assay that combines known and novel COVID-19 biomarkers. The prognostic value of the peptide panel assay should be prospectively assessed in larger patient cohorts for future support of clinical decisions, including evaluation of sample-flow in routine setting. The possibility to objectively classify COVID-19 severity can be helpful for monitoring of novel therapies, especially in early clinical trials.

The clinical presentation of COVID-19 is extremely diverse, ranging from asymptomatic infection to fatal disease, and can change rapidly. Timely assignment of the appropriate level of care substantially improves outcome in COVID-19. Objective and easy-to-apply tools to anticipate the patient's risk of deterioration, maximum disease severity and outcome based on validated biomarkers are fundamentally required, particularly in situations with overstrained healthcare resources. Risk assessment, as informed by established ICU outcome predictors, such as APACHE II or SOFA, as well as prognostic markers that depend on established clinical chemistry, have so far proven to be of limited predictive value in COVID-19. Moreover, the pandemic situation has prompted an unseen amount of repurposing attempts of existing antiviral and immunomodulatory drugs. However, small and rapidly conducted clinical trials can fail to yield reliable results with regard to clinical benefit and patient safety. Plasma and serum proteome studies have recently shifted to the centre stage to provide added value in COVID-19 patient classification and outcome prediction. Proteomic assays could overcome limitations surrounding the accuracy of early clinical testing. However, proteome analyses are thus far restricted to research settings, as the technology employed in discovery proteomics does not meet the requirements of technical stability and ease of implementation as required in the routine laboratory. In order to bridge the gap between research based discovery proteomics and the clinical application of a proteomic marker panel, we used proteomic datasets recorded in a deeply phenotyped COVID-19 patient cohort. We select a panel of 50 peptides derived from 30 proteins whose functions are associated with the COVID-19 host response and which can classify disease severity in COVID-19. Assembled on the basis of observational criteria, our panel contains a set of established clinical markers, but is also based on new protein markers that are not in use in the routine so far. We then develop and analytically validate a scalable and standardised proteomic panel assay that may be performed on instrumentation common in certified laboratories. Applying the assay to two independent cohorts, we demonstrate accurate disease classification, and show that the marker panel is prognostic about outcome. There is value in using the human plasma proteome in severity classification, risk assessment and outcome prediction in COVID-19. Missing so far is a translation of this research evidence into a routinely applicable assay. We show that a proteomic marker panel, which predicts survival in COVID-19 with high accuracy, can be used in routine laboratory testing. The described proteomic marker panel has the potential to substantially improve clinical risk assessment for patients with COVID-19 by translating discovery proteomics findings to patient care.

The invention will now be further described with reference to the Examples which are present for the purposes of reference only and are not to be construed as limitations on the invention.

### EXAMPLE 1: CHARACTERIZATION OF PLASMA PROTEOME AND PATIENT RESPONSE TO COVID-19 DISEASE

1. Venous blood was collected from patients suspected or diagnosed to have COVID-19

2. EDTA plasma was prepared from the collected blood samples using standard hospital pathology laboratory protocols.

3. Plasma samples were prepared for mass spectrometry analysis by spiking with heavy isotope-labelled peptides shown in Table 1 and digestion with trypsin to release tryptic peptides from plasma proteins for analysis on the targeted LC-MS/MS platform.

4. LC-MS/MS analysis was performed on the prepared plasma samples by measuring tryptic peptides. This occurred on a pre-configured targeted LC-MS/MS system which was “programmed” according to the specifications in Table 2 to detect and quantify a specific set of peptides in Table 1.

5. Tryptic peptide concentration in μg/ml or ng/ml was determined for all 52 analytes using heavy isotope-labelled internal standards and synthetic reference peptides at known concentration as shown in Table 1.

6. Tryptic peptide concentration with a patient's age was used as input in a linear regression statistical model to generate an overall risk score for each individual patient.

7. The test results were sent to a treating clinician in order to make a treatment decision, taking other clinical readouts and their judgment into account.

### EXAMPLE 2: COVID-19 ANTIGEN CALIBRATION CURVE PREPARATION

(i) solution preparation:
Peptide Storage Solvent: 50% acetonitrile, 50% ddH₂O.
Denaturation Buffer: 8M Urea, 100 mM ammonium bicarbonate, 50 mM DTT.
Alkylation Agent: 100 mM IAA.
100 mM ammonium bicarbonate.
Surrogate Matrix: 40 mg/ml bovine serum albumin in ddH₂O.

(ii) Internal standard Spike Solution (Peptide Mix A)
To an Eppendorf labelled as “Internal Standard Mix”, 20 μl each of all 52 internal standard peptide stock solutions were added.

Final volume of pooled IS mix was 1.04 ml.
A 5 ml Eppendorf was labelled with “Peptide Mix A”.
To “Peptide Mix A”, 200 μl of “Internal standard Mix” was added.
“Peptide Mix A” was dried down to completion and stored at −20° C. until calibration sample digestion.

(iii) Calibration Curve Preparation
A 5 ml Eppendorf was labelled with “Cal 0”.
To “Cal 0”, 55 μl of each of the 52 native peptide stock solution was added. Total volume of the native pool was 2.86 ml.
A set of ×20 1.5 ml Eppendorf tubes was labelled with “Plasma Cal 1”-“Plasma Cal 18”, “Plasma IS Only” and “Plasma Blank”.
A 2^{nd} set of ×20 1.5 m Eppendorf tubes was labelled with “BSA Cal 1”-“BSA Cal 17”, “BSA IS Only” and “BSA Blank”.
To all ×40 tubes that have been labelled during steps 3.3. & 3.4, 650 μl of peptide storage solvent was added.
To “Plasma Cal 1”, 650 μl of “Cal 0” was added and the sample was mixed well.
To “Plasma Cal 2”. 650 μl of “Plasma Cal 1” was added and mixed well.
The serial dilution process was continued until “Plasma Cal 18” was reached.
650 μl of sample was removed from “Plasma Cal 17” and discarded to achieve required concentration.
Nothing was added at this point to either “Plasma IS only” or “Plasma Blank”.
The serial dilution process detailed in steps 3.6-3.10 was repeated using the 2^{nd} set of tubes that were labelled in step 3.4 (“BSA Cal 1 . . . ”).
All samples were dried down to complete dryness. C0 stock was not dried down and remaining sample was kept in the fridge for repeat curves.

(iv) Calibration Sample Tryptic Digestion
Denaturation buffer was thawed.
To all ×19 “Plasma Cal . . . ” samples, equal volumes of pooled human plasma was added.
To all ×19 “BSA Cal . . . ” samples, equal volumes of 40 mg/ml BSA was added.
To the dried “Peptide Mix A”, denaturation buffer was added.
“Peptide Mix A” was vortexed in order to mix well.
“Peptide Mix A” was left to mix gently for 10 minutes in order to fully solubilise internal standards.
To “Plasma Cal Blank” & “BSA Cal Blank”, equal volumes of denaturation buffer was added.
To all other calibration samples, equal volumes of “Peptide Mix A” was added.
Samples were spun briefly to ensure all solution was collected at bottom of tubes.
Samples were left mixing at 30° C. for 1 hour.
Equal volumes of alkylation agent was added to samples.
Samples were mixed briefly and then spun briefly.
Samples were incubated at room temperature in the dark for 30 minutes.
Equal volumes of 100 mM ammonium bicarbonate was added to the samples.
Samples were then spun briefly.
Mass spectrometry grade trypsin was made up to a final concentration of 0.1 μg/μl in 100 mM ammonium bicarbonate.
Equal volumes of trypsin solution was added to all samples.
Samples were incubated at 37° C. overnight with gentle mixing.
The following morning, equal volumes of formic acid was added to all samples to quench the digest and prepare samples for SPE clean up.
Samples were mixed and proceeded to SPE processing.

### EXAMPLE 3: A MULTIPLEXED, TARGETED LIQUID CHROMATOGRAPHY MASS SPECTROMETRY-BASED PROTEIN PANEL ASSAY DETERMINES COVID-19 DISEASE SEVERITY AND IS PROGNOSTIC ABOUT OUTCOME

Abstract

Global healthcare systems continue to be challenged by the COVID-19 pandemic, and there is a need for clinical assays that can both help to optimize resource allocation and be used to monitor clinical trials. Several recent studies reported a huge potential for plasma and serum proteomics in classifying COVID-19 disease severity and to provide accurate outcome prediction weeks in advance. However, these investigations are so far based on shotgun and discovery proteomic platforms using nanoflow chromatography and relative quantification, which are difficult to implement in clinical settings and routine laboratories. Here, we present an absolute quantitative proteomic panel assay for the assessment of COVID-19 disease severity and outcome. For the ease of implementation, the assay is based on analytical flow rate chromatography and multiple reaction monitoring (LC-MRM), and runs on equipment available in routine and CLIA laboratories. We demonstrate classification of severe COVID-19 patients according to treatment choices in two cohorts. Moreover, we show that the panel assay substantially outperforms established risk assessments such as the Charlson comorbidity Index, the SOFA score, and the Apache II in predicting COVID-19 outcome. Our study hence shows that the combination of discovery and targeted plasma proteomics based on analytical-flow rate chromatography has become a powerful platform that facilitates the rapid translation of proteomic findings into routinely applicable clinical assays.

Introduction

COVID-19 continues to challenge healthcare systems worldwide despite vaccination efforts and novel treatments. This is particularly apparent in areas with limited vaccine uptake or supply. Currently, the outlook remains uncertain even in countries with high vaccination rates as the immunity conferred by the vaccines appears to diminish over time (2-5) and SARS-COV-2 variants with varying capacity to evade vaccine induced immunity continue to emerge (2, 6-8).

Biomarker tests that can classify patients, predict disease severity and are prognostic could help optimise resource allocation, specifically during a pandemic (9-11). Indeed, clinical manifestation of COVID-19 is highly variable, which does create challenges in timely clinical decision making. For instance, ‘happy hypoxia’ describes situations where COVID-19 patients report minor impairments, while molecular indicators such as blood oxygen levels, indicate they are, in fact, severely ill (12). Furthermore, in situations when healthcare systems reach maximum capacity, prognostic markers could support difficult clinical decisions, for instance, to navigate through triaging situations (13). Prognostic and disease severity tests could further help to increase the likelihood of success and accelerate clinical trials, by improving the assessment of treatment efficacy of COVID-19 therapies or stratify patient populations that are to be included in the trials. Indeed, COVID-19 prompted the rapid repurposing and development of new treatments. However, during a pandemic, there is pressure to conduct trials in a timely manner. Furthermore, specifically in ICU settings, study cohorts are limited in size, and when underpowered, clinical trials can lead to false positive and false negative results (14)(15). So far however, the reliability of several risk assessment scores conventionally used in ICU settings, such as the Acute Physiology And Chronic Health Evaluation (APACHE II) and Sequential Organ Failure Assessment (SOFA) scores, appear to be limited for COVID-19 cases (16). Moreover, combinations of generic clinical readouts, e.g. blood oxygen saturation, interleukin-6 concentration, have been considered for COVID-19 outcome prediction at various disease severity stages (Vatansever and Becer 2020), however, several predictive models based on clinical parameters and routine tests were reported to be vulnerable to bias and not suitable for the clinic (17,18).

Protein biomarker signatures from plasma present a promising alternative. Proteomic datasets have repeatedly been successful to classify and predict COVID-19 severity and outcome (9, 10, 16, 19-21). For instance, we recently presented a proteomics biomarker signature that stratified COVID-19 severity grades and successfully predicted disease progression and outcome, such as survival of hospitalised COVID-19 patients (13, 22, 23). Furthermore, specifically early in the pandemic, proteomics successfully characterized the antiviral host response, which greatly improved our understanding of the COVID-19 disease (9, 10, 16, 19-21, 23).

The success of shotgun proteomics in the clinical routine is so far however limited for technical and economic reasons. Most of the research platforms require a high degree of expert knowledge to use and they are also susceptible to interference and batch effects. Moreover, with only recent exceptions (15,22), most discovery proteomics platforms make use of nano-flow chromatography, which limits the throughput of proteomics while creating high maintenance efforts. The objective of this study was to translate selected biomarkers from this discovery based approach into a routinely applicable proteomics platform that could be deployed for clinical use within existing regulatory frameworks and on broadly available analytical instruments. Triple quadrupole mass spectrometers coupled to high flow liquid chromatography were the optimal choice for rapid test development and deployment as they are used in the clinic in other areas, for instance rare diseases, newborn screening and steroid hormone analysis (24-27). Further, they are widely available in large hospital laboratories, diagnostic laboratories and contract research organizations. Biomarker tests developed on this platform can be accredited to existing regulatory standards in GCP, ISO:17025, ISO:15189 and CLIA environments, standardised and transferred across different instruments, manufacturers and laboratories, and thus deployed at scale rapidly. Triple quadrupole mass spectrometry-based tests are cost effective to run at scale as the sample preparation is amenable to automation, consumables costs are typically <£5 per test and the instrument uptime is typically >95%. The tests also integrate with existing workflows at clinical and analytical laboratories.

We have mined discovery proteomics data from COVID19 patients, and selected peptides that are informative about COVID19 disease progression and severity. The biomarkers were chosen for i) being prognostic of remaining duration of hospitalisation, disease aggravation, or being differentially concentrated in plasma depending on the treatment escalation level, used as a measure of disease severity, and ii) participating in biological processes that contribute to COVID-19 pathology, and iii) to be technically and analytically suitable for the assay. The COVID-19 severity biomarkers chosen include proteins that function in inflammation (e.g. C-reactive protein), coagulation and vascular dysfunction (e.g. von Willebrand factor), complement cascade (e.g. Complement C1q subcomponent subunit C) and diverse biological processes detected to be altered by COVID-19 (e.g. Cystatin C). We then established a targeted proteomics assay utilising multiple reaction monitoring (MRM) data acquisition mode. Employing calibration curves with synthetic reference and stable isotope labelled internal standards, the assay provides absolute quantification of 50 surrogate tryptic peptides arising from 30 plasma proteins (Table 5). These includes proteins that function in inflammation (e.g. C-reactive protein), coagulation, vascular dysfunction (e.g. von Willebrand factor), complement cascade (e.g. Complement C1q subcomponent subunit C) and other biological processes detected to be altered by COVID-19 (e.g. Cystatin C, which is a known marker of kidney dysfunction). We have validated the assay technically, and implemented it in two analytical laboratories employing two triple quadrupole LC-MS/MS platforms from different vendors. The assay was applied to two cohorts with a total of XX patients and XX samples. We demonstrate that the presented MRM assay captures host response to SARS-COV-2 and, based on it, classifies and predicts COVID-19 disease severity and is predictive about outcome in severely ill individuals. We found that the biomarker panel is predictive about survival weeks before outcome and outperforms several commonly used risk assessment scores.

Results

Peptide Selection and Optimisation of an MRM Based, Targeted COVID-19 Biomarker Assay.

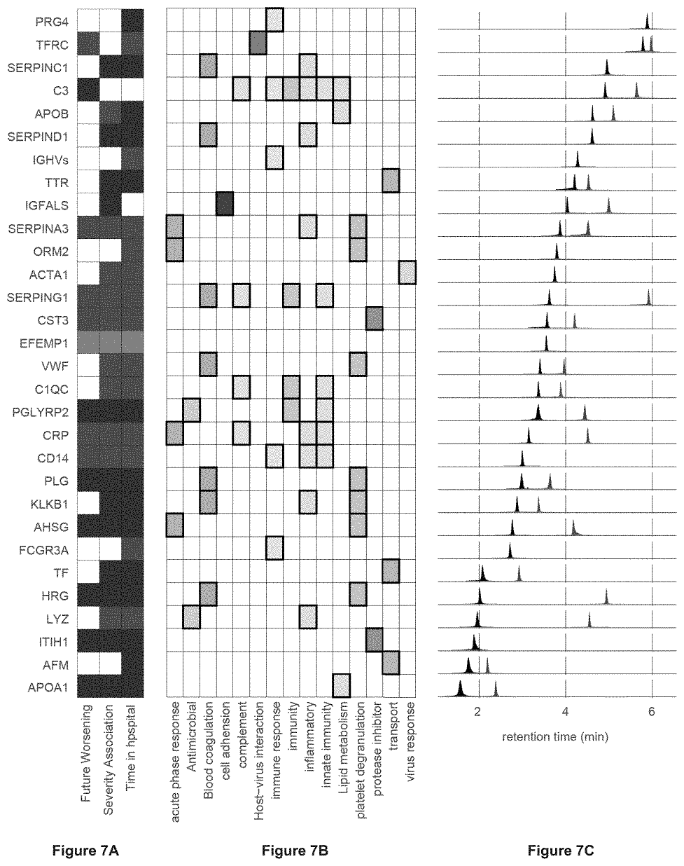

To select peptides for a COVID-19 biomarker panel assay, we used shotgun plasma proteomic data recorded on a deeply phenotyped COVID-19 patient cohort treated at Charit\tilde{e} Universitätsmedizin Berlin (13,22). On the basis of xx proteomes recorded for xx patients, we selected 50 proteotypic peptides that corresponded to 30 plasma proteins. These peptides were chosen based on their ability to predict the future worsening of COVID-19 over time, the remaining time in hospital for respective COVID-19 patients, which in turn serves as a treatment-insensitive proxy for COVID-19 severity (FIG. 7 a) (13, 28) (Table 5). Indeed the proteins chosen only over their predictive value, were found to be associated with functions associated with COVID-19, in particular the innate immune system and the coagulation cascade, and increased in abundance depending on the treatment escalation level, expressed on the WHO ordinal scale (FIG. 7b) (13, 16, 19, 22, 29).

To quantify the 50 proteotypic peptides, we obtained synthetic reference standards. For each peptide 2 synthetic standards were manufactured: 1) with a natural isotope distribution (‘native’) and 2) with a C-terminal stable isotope-labelled (SIL) amino acid (either [13C6,15N2]-lysine or [13C6,15N4]-arginine) to act as an internal standard. Internal standards contained a short tryptic tag to account for the digestion efficiency (Table 5). The native peptides were employed to optimise liquid chromatography-mass spectrometry (LC-MS/MS) data acquisition method and quality of the Q1/Q3 (MRM) transitions. To select optimal MRM transitions for each peptide, we first predicted the transitions (consisting of several precursor ion charge states and respective product ions) using Skyline v21.1.0.146 (30). We infused each native peptide solution into the LC-MS/MS system and selected 1 precursor ion per peptide with the highest relative intensity and 5 most abundant product ions for collision energy optimization which was also provided by Skyline. From these 5 product ions, we ultimately selected 2-5 experimentally optimised ion transitions per native peptide based on i) highest relative signal intensity, ii) optimal chromatographic peak shape and iii) absence of interfering signals. Product ions of <300 Da were excluded where possible to ensure specificity. Precursor and product ion-matched SIL internal standard transitions were included. Lastly, all selected transitions were combined into one dynamic MRM method, which was subsequently analytically validated. In the designed method, the most abundant transition for each peptide is used for quantification and 1-4 less abundant transitions are used as qualifiers (Tables 6 & 7). In order to establish the assay on a chromatographic system that could be run in a routine setting, we chose analytical flow-rate (800 μl/min) reversed phase chromatography using an 1290 Infinity II (Agilent) binary pump. The selected 50 peptides were well distributed along a 8.6 minute linear gradient, and quantifiable with the chosen total runtime of 10 minutes (FIG. 7c). For data acquisition, the LC was online-coupled to a triple quadrupole mass spectrometer (Agilent 6495C). For a cross platform comparison, the assay was also established on another triple quadrupole MS (Sciex 7500) coupled to ExionLC AD UHPLC system (SCIEX, UK; Methods; Table 7).

Analytical Validation of the Peptide Panel MRM Assay.

To evaluate the general applicability of the method for a clinical assay we tested performance of the selected peptides/transitions with respect to intra- and inter-batch repeatability, linearity, limits of quantification, accuracy, and potential matrix effects.

First, we determined the intra- and inter-batch repeatability. We calculated the coefficient of variation (CV) between three independently prepared replicate calibration curves.

These were constructed from serial dilutions of native peptide standards in BSA, covering a concentration range ˜5×105, and measured in technical pentuplicates (N=15). We used BSA as a surrogate matrix to test the analytical performance achieved on the standards in the absence of the endogenous plasma peptides (31) and achieved a median inter-batch CV of 10.9% across low (LLOQ), medium ((LLOQ+ULOQ)/2) and high (ULOQ) concentration points (Table 8). CV was determined from the response ratios, calculated by dividing native peptide area by internal standard peptide area. Additionally, we determined the limits of quantification as the highest and lowest concentration points on the linear calibration curve where the CV of the inter-batch repeatability was 520%. 37/50 peptides fulfilled the ≤20% CV cutoff requirement. Since analytical validation requirements for clinical assays are purpose and context dependent, and are influenced by the magnitude of change of target analyte levels in control versus disease samples, we subsequently expanded the CV cutoff to 540%. This enabled us to determine LOQ of 10 additional peptides and prevented missing values in downstream data analysis. Thus, we eventually determined limits of quantification and generated calibration curves for 47 peptides, with a median LLOQ of 1.6 ng/ml. Obtained calibration curves also showed excellent linearity within the determined LOQs (R2>0.99) and typically spanned 3-4 orders of magnitude (Table 8).

As LC-MS measurements can suffer from matrix effects (32), we next evaluated how quantification in surrogate matrix (BSA) compares to human plasma. We compared the slopes obtained from calibration samples measured in commercial human plasma samples (manufacturer), with those measured in the surrogate matrix. 41/47 quantified peptide biomarkers that passed the above described validation showed no statistically significant matrix effects when comparing the slopes between matrices (P>0.05) as expected for an assay using SIL internal standards (33,34). 6 peptides (AADDTWEPFASGK (SEQ ID No: 35), ASDTAMYYCAR (SEQ ID No: 50), ATEHLSTLSEK (SEQ ID No: 25), EQLSLLDR (SEQ ID No: 12), GDVAFVK (SEQ ID No: 13), IADAHLDR (SEQ ID No: 29)) differed significantly and matrix factor (slope plasma/slope BSA×100%) was calculated. The matrix factor was within the acceptable +/−20% limits and these peptides were included in the final method.

To test if peptide quantities from actual patient samples would be covered within the (linear) range of the calibration curves, we performed absolute quantification in plasma samples obtained from COVID-19 patients (pooled COVID-19 patient samples of different WHO severity grades; see methods). Absolute quantification was performed by calculating the relative response ratio (peak area ratio of native peptide over its corresponding ISTD peptide) in pooled samples, followed by absolute concentration interpolation from the above described BSA calibration curve. The peptide concentrations obtained from patient samples were covered within the determined linear range of the assay and were measured with a median accuracy of 97.3% across low (LLOQ), medium ((LLOQ+ULOQ)/2) and high (ULOQ) concentration points (Methods; Table 8).

Diagnostic Performance of the Peptide Panel Assay in an Early COVID-19 Cohort.

Next, we assessed how the absolute concentration of the selected potential biomarkers changed as a function of the COVID-19 treatment escalation level, a proxy for disease severity, as expressed by the WHO ordinal severity scale. To establish this relationship, we applied the MRM assay on a COVID-19 cohort that was also analysed with a discovery proteomics platform (23). This cohort was sampled as citrate plasma (in contrast to EDTA plasma used in the cohorts employed to select the peptides), and included healthy controls, as well as samples from patients hospitalized during the first wave of the pandemic between March 2020 and XX with mild to severe forms of the disease (23).

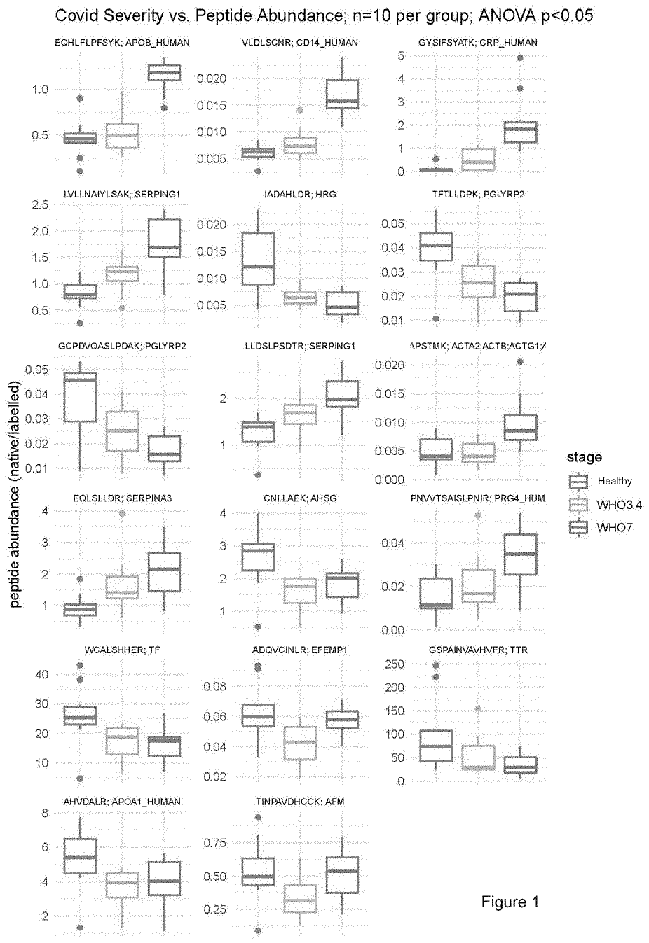

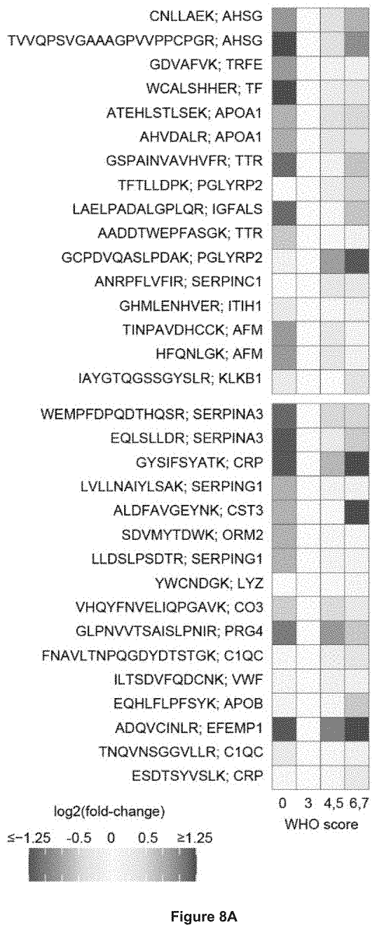

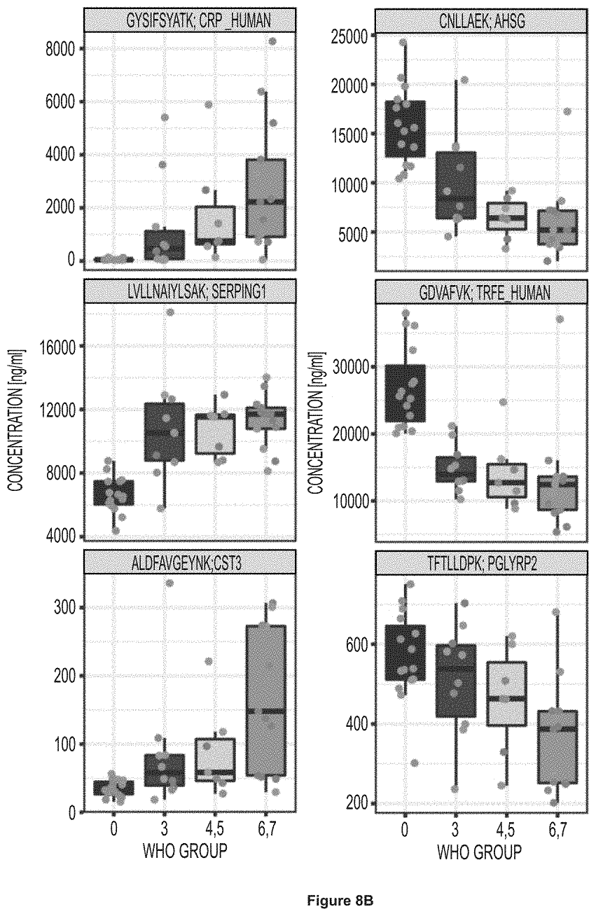

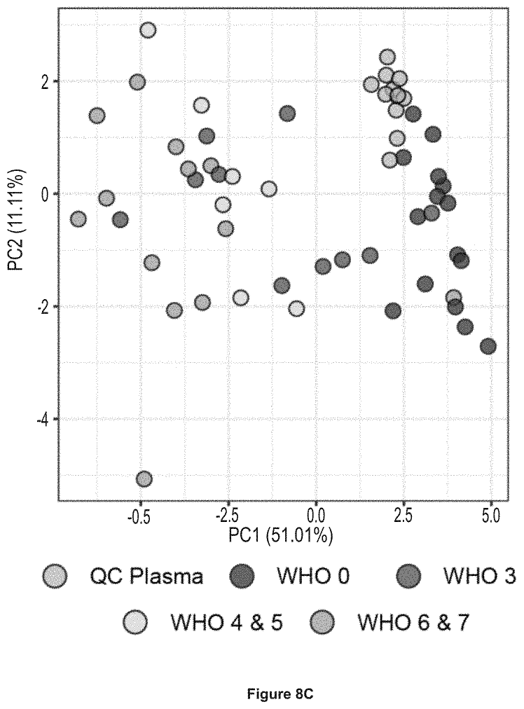

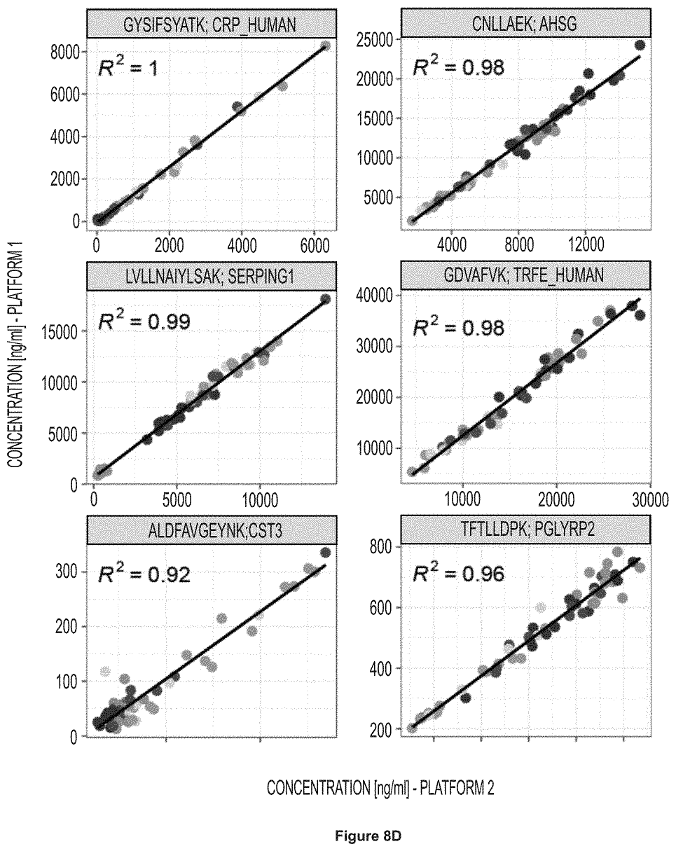

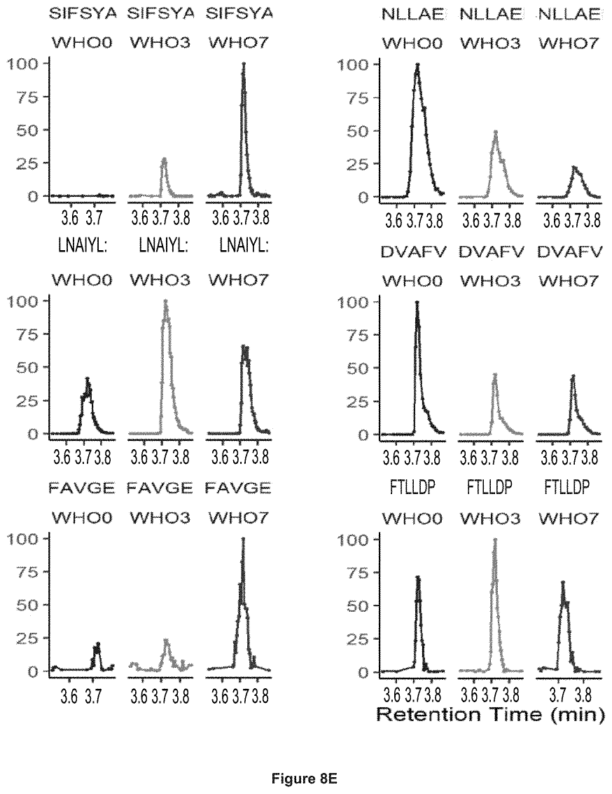

For robust and reliable sample preparation, we employed the recently presented procedure that enables tryptic digest and solid phase extraction in a semi-automated way (22). 40/50 peptides could be reliably detected and quantified on the Agilent 6495c LC-MS/MS platform (Table 9). The concentration of 32 of these both up- and down-regulated peptides changed with the severity of the COVID-19 disease according to treatment escalation: i.e. from uninfected (WHO 0) to mild (WHO3), moderate (WHO 4, 5) and severely (WHO 6, 7) COVID-19 affected individuals (FIG. 8a, Supplementary FIG. 1, P<0.05). Peptide abundance profiles could be roughly split into three groups (FIG. 7a). In group i) markers where levels are different between healthy and COVID-19 infected individuals, and further follow their respective trend with increasing disease severity such as peptides derived from the acute phase proteins CRP and AHSG (FIG. 7a, FIG. 7b, top panel), ii) peptides that mainly changed between infected and uninfected individuals, such as peptides from the complement related protein SERPING1 or the iron-binding protein TF (FIG. 7a, FIG. 7b, middle panel), and iii) markers that are particularly changing in severe forms of COVID19, such as the kidney- and inflammation-marker CST3 and the innate immune response protein PGLYRP2 (FIG. 7a, FIG. 7b, bottom panel). Furthermore, despite the small scale of this cohort, obtained peptide-abundance profiles could overall stratify COVID-19 severity using principal component analysis (FIG. 7c). Thus, we successfully quantified 40 peptides in COVID-19 patient plasma samples by high-flow LC-MRM, of which 32 were differentially concentrated in COVID-19 severity grades from healthy controls to increasing treatment escalation levels.

Analytical Cross-Platform and Cross-Laboratory Validation.

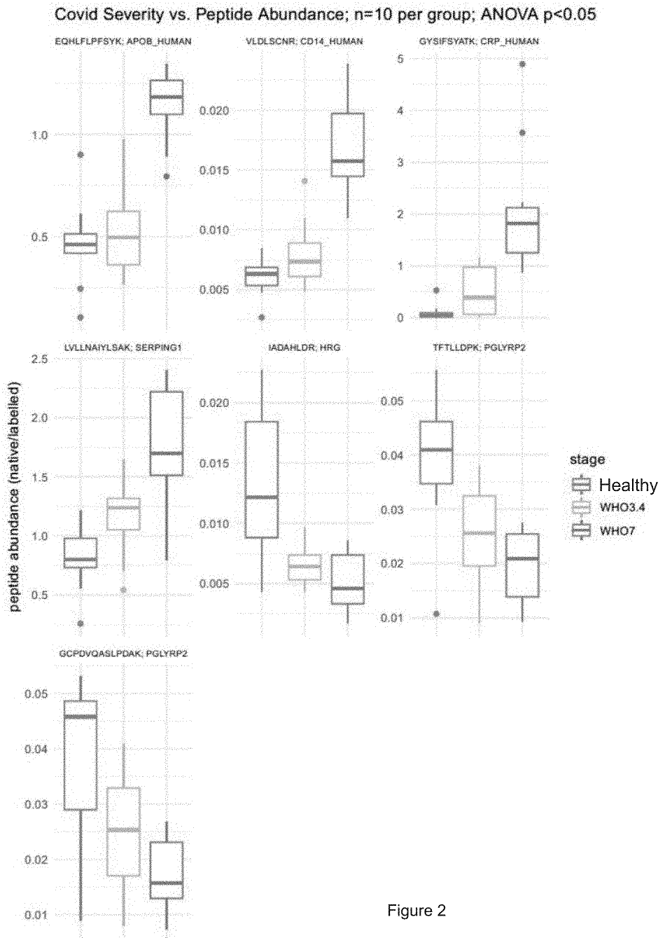

To evaluate transferability of the assay, samples from the above described cohort were further employed for a cross-platform, cross-laboratory validation of the LC-MS/MS assay. Post-digested samples were analyzed on a triple-quadrupole platform from a different vendor, with independently optimised MRM transitions (Table 7), in a different laboratory. For the majority of selected peptides, we obtained an excellent correlation between the concentration measured in respective samples on both instruments (FIG. 7d, Supplementary FIG. 2). 6 peptides were close to the quantification limit in this cohort and sample matrix (citrate plasma), leading to higher variance that translated into a poor correlation (R2<0.6) between both platforms. Additionally, the peptide ESDTSYVSLK (SEQ ID No: 20) suffered from one outlier, while the overall correlation between both measurements was high.

Similarly, the absolute quantities obtained for individual peptides were highly similar, with some exceptions where there was a high correlation, but some discrepancies in the obtained absolute concentration, suggesting differences in the employed calibration curves (see for instance the peptide CNLLAEK (SEQ ID No: 27) in FIG. 7d).

Overall, as we detected and quantified the majority of peptides on both platforms with high precision, the peptides that differentiate between different stages of COVID-19 can be quantified on both platforms, demonstrating the general applicability of the assay independent of the employed analytical platform.

Severity Stratification and Prediction of Disease Progression in a Longitudinal COVID-19 Cohort.

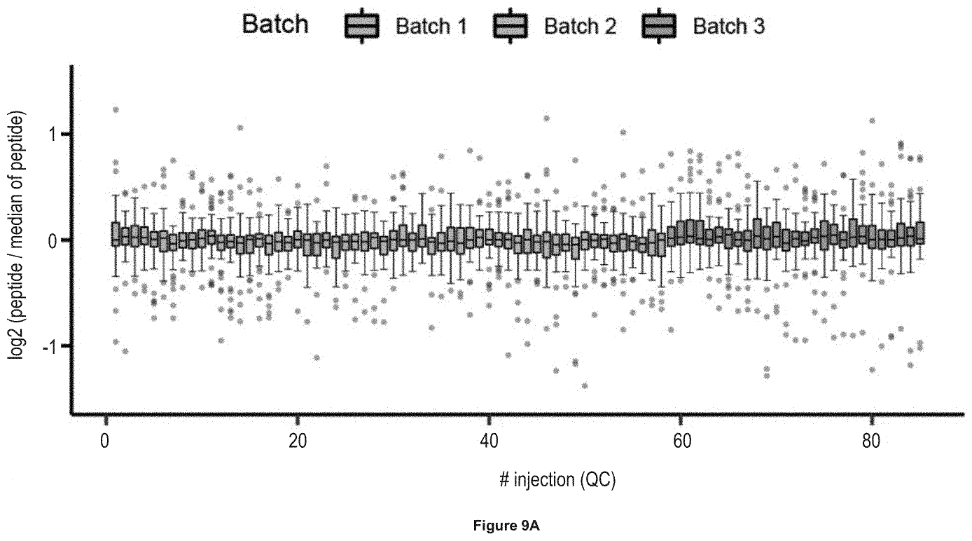

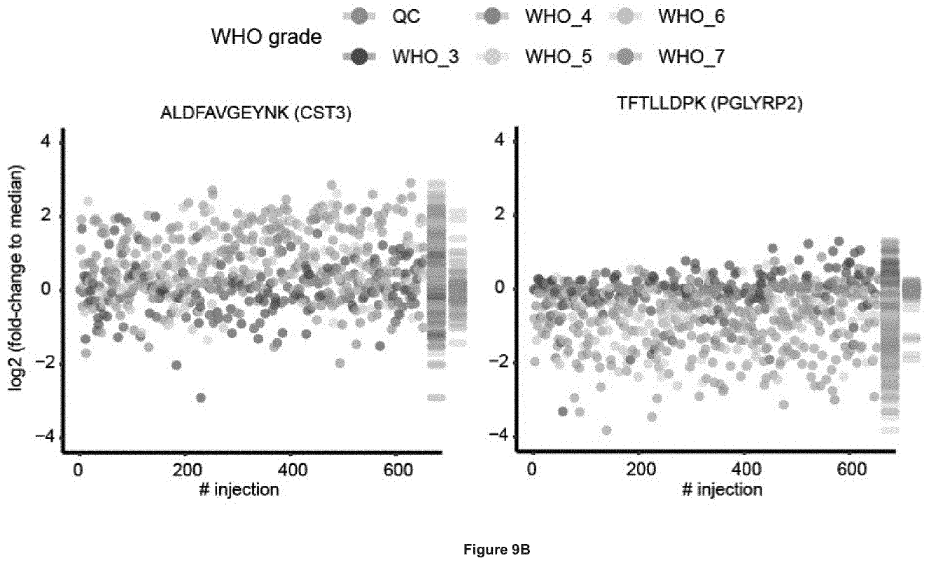

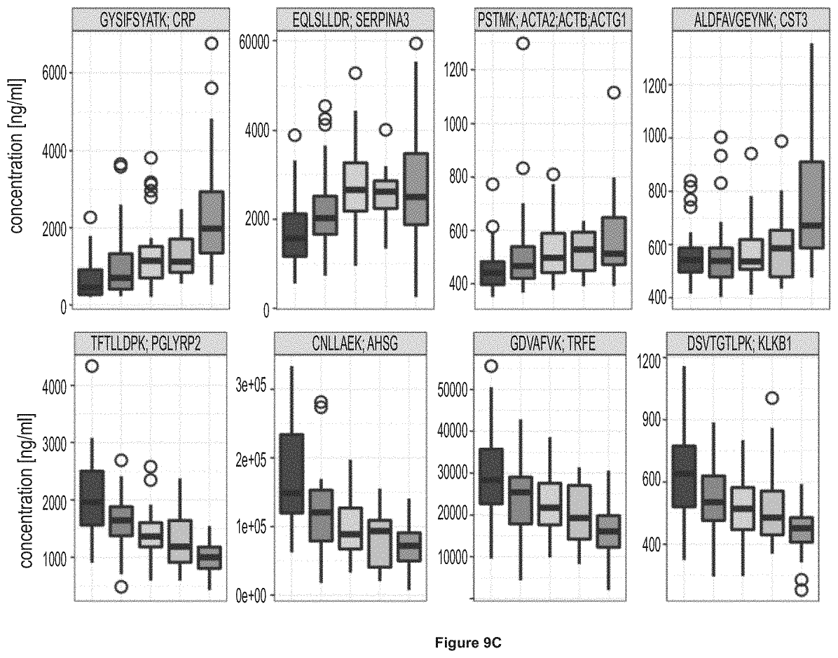

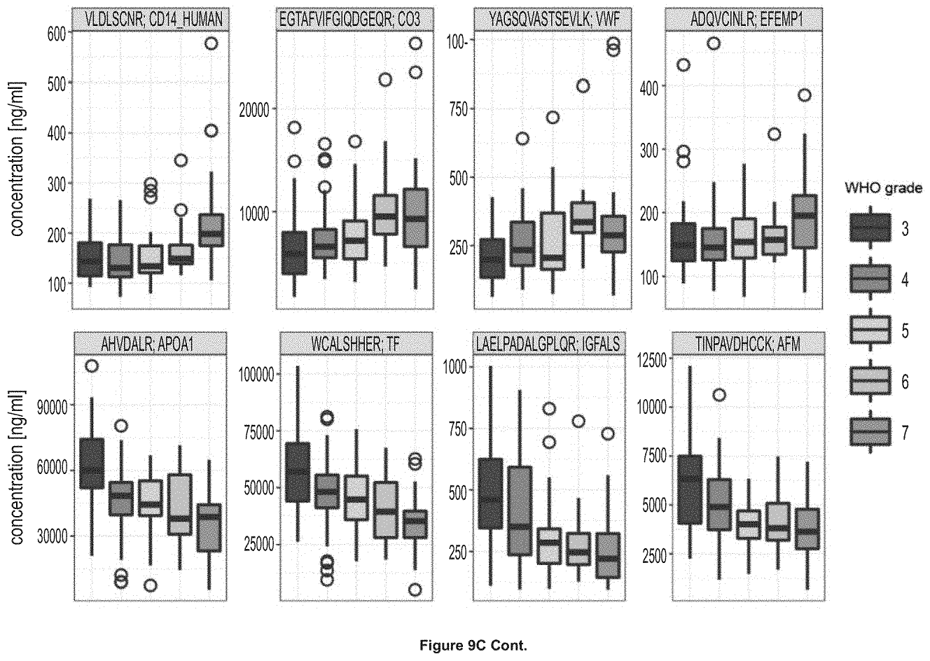

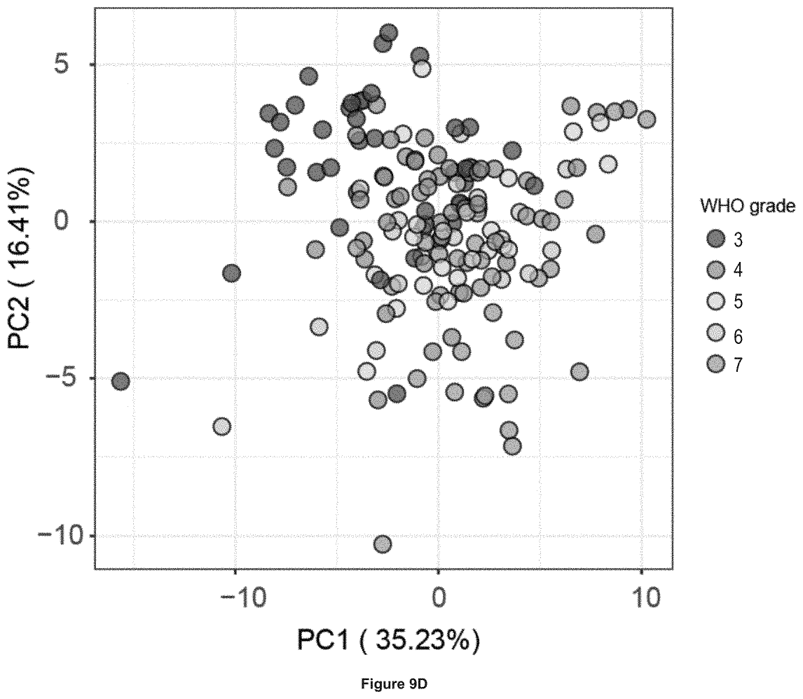

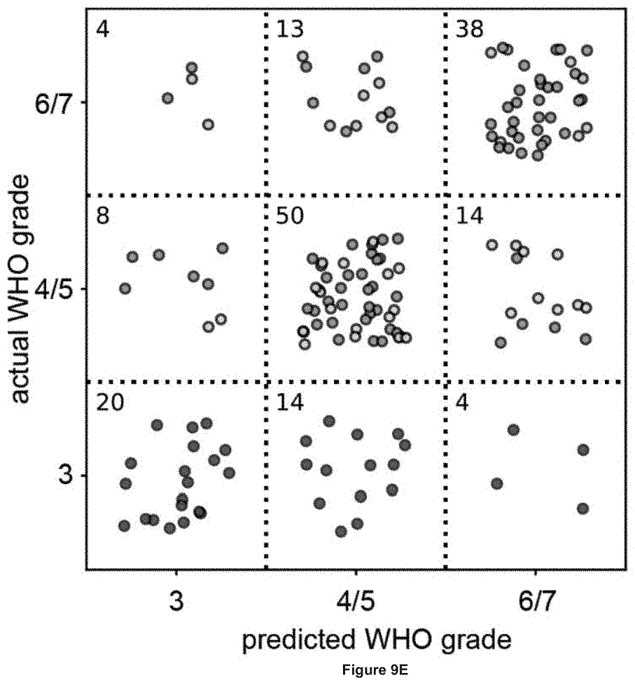

After establishing that the assay successfully captured hallmarks of COVID-19 disease in a small patient cohort, we next measured a large, longitudinal collection of samples obtained during the second wave of the pandemic in Germany. This cohort was selected as i) it provided a large number of samples, and thus more statistical power to evaluate the potential of the MRM assay to stratify disease severity, and ii) the longitudinal nature of the study, which allowed us to assess the predictive value of the MRM panel. Reassuringly, despite the large number of samples acquired (n=655 including quality controls) split over three batches and measured over 10 days, variation was low and no significant batch-effect was observed (FIG. 9a). Moreover, the abundance of peptides corresponding to CST3 and PGLYRP2, two markers we found characteristic for severe forms of the disease in the first analyzed cohort (FIG. 7b, bottom), again clearly differed between different WHO grades (FIG. 8b).

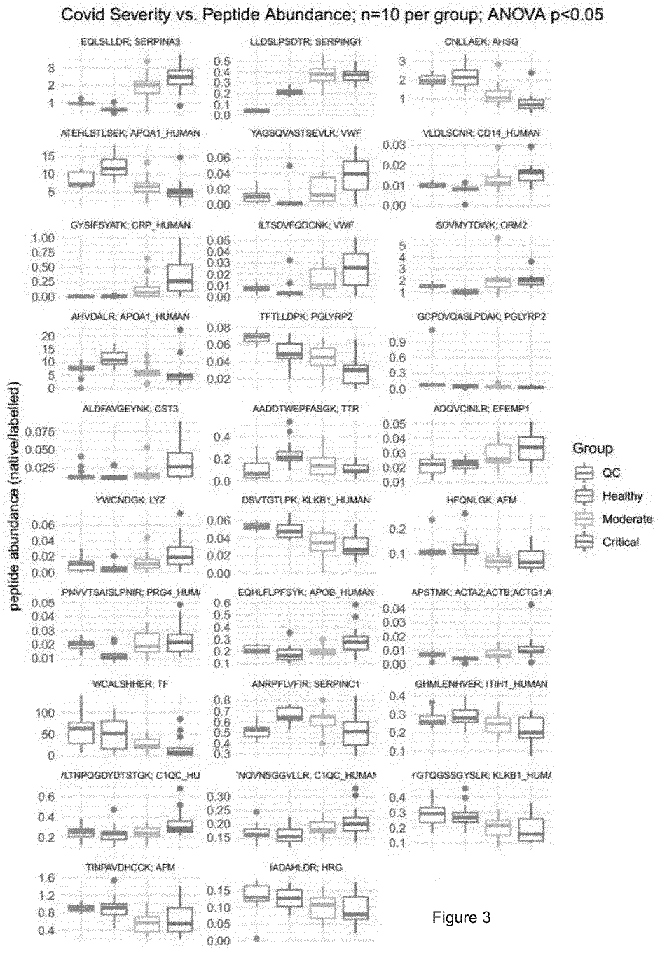

To evaluate the ability of the whole MRM panel to differentiate COVID-19 severity, we first analyzed the earliest sample obtained for each patient of this cohort (n=165). We were able to reliably detect and quantify 47/50 peptides, and the majority (33 peptides, 11 up-regulated, 22 downregulated) had a significant trend between patients according to the WHO ordinal outcome scale for clinical improvement with patients capturing a WHO score from relatively mild (WHO 3) to very severe cases (WHO 7) (Supplementary FIG. 3). Considering the most differentially up- and down-regulated peptides (FIG. 8c, only one peptide per protein considered), the assay clearly captures differences between different WHO grades, used as a proxy for disease severity. Similarly, dimensionality reduction of all reliably detected peptide abundance profiles using principal component analysis shows that the assay stratifies individuals according to their WHO score (FIG. 8d).

To further explore the possibilities to classify COVID-19 severity with the MRM assay, we constructed a support vector machine trained to differentiate between three different treatment groups: WHO grade 3 (mild COVID-19, hospitalised, but no oxygen therapy necessary), WHO grade 4/5 (severe COVID-19, hospitalised, non-invasive oxygen therapy) and WHO grade 6/7 (severe COVID-19, hospitalised, mechanical ventilation).

The data was split in a training and a validation set in a cross-validated manner. The SVM successfully predicted the WHO grade in the prediction validation set (FIG. 9). This performance strengthens the observed significant differentiation of WHO grades based on single peptides and shows that combinations of these peptides are capable of separating WHO severity groups.

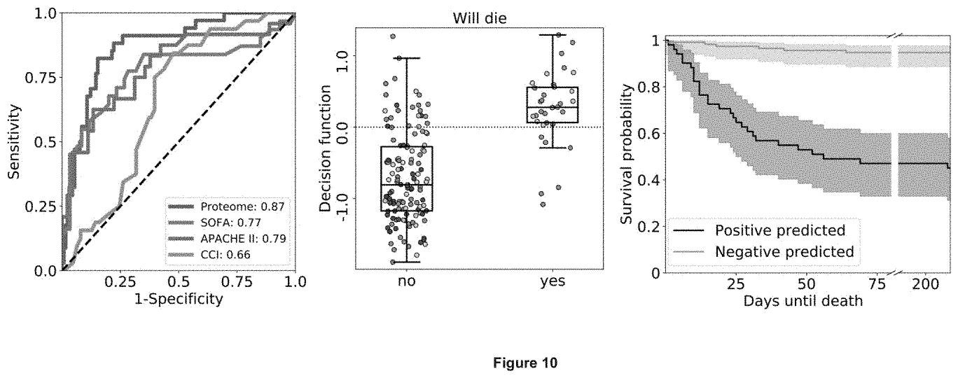

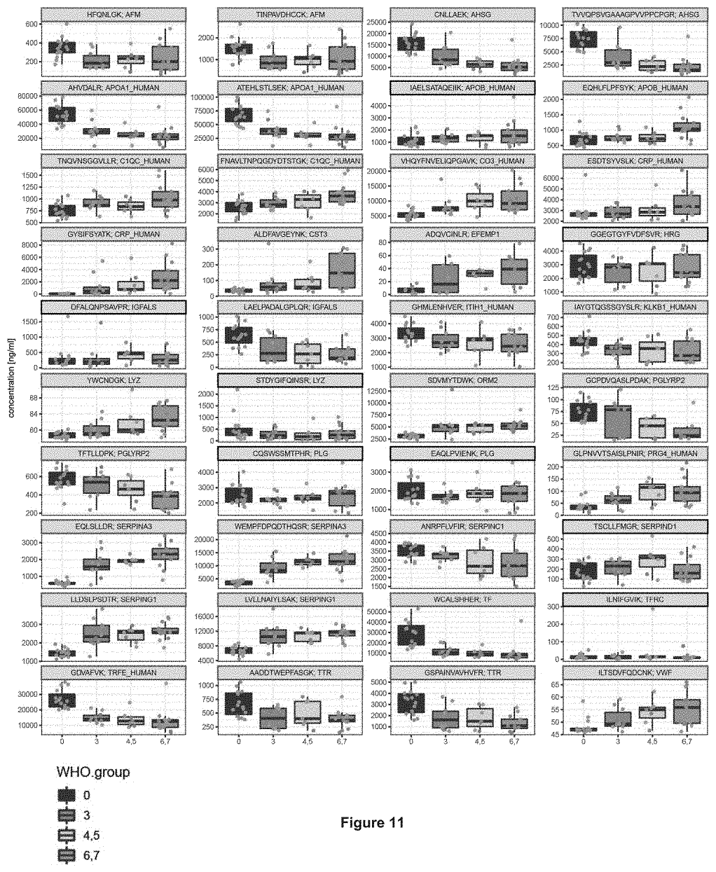

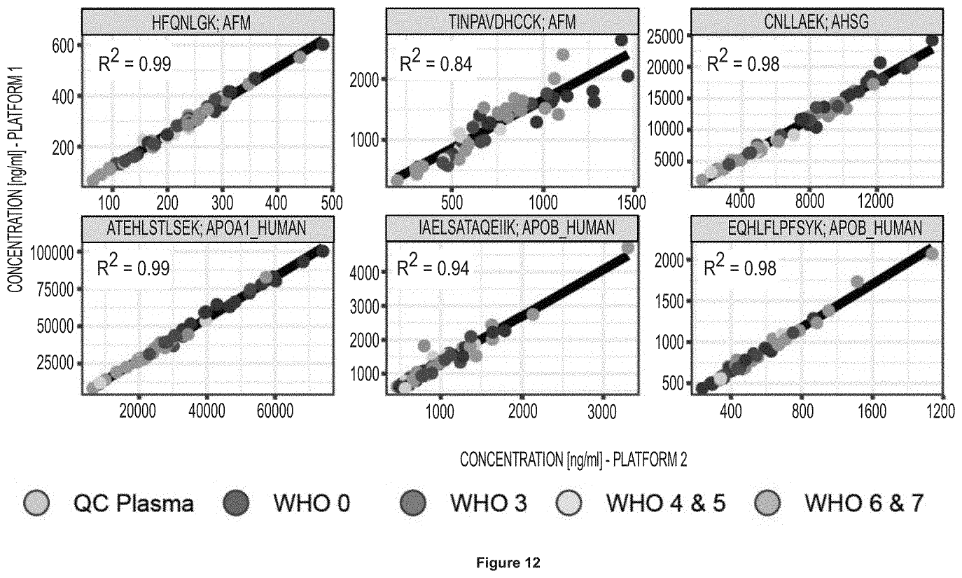

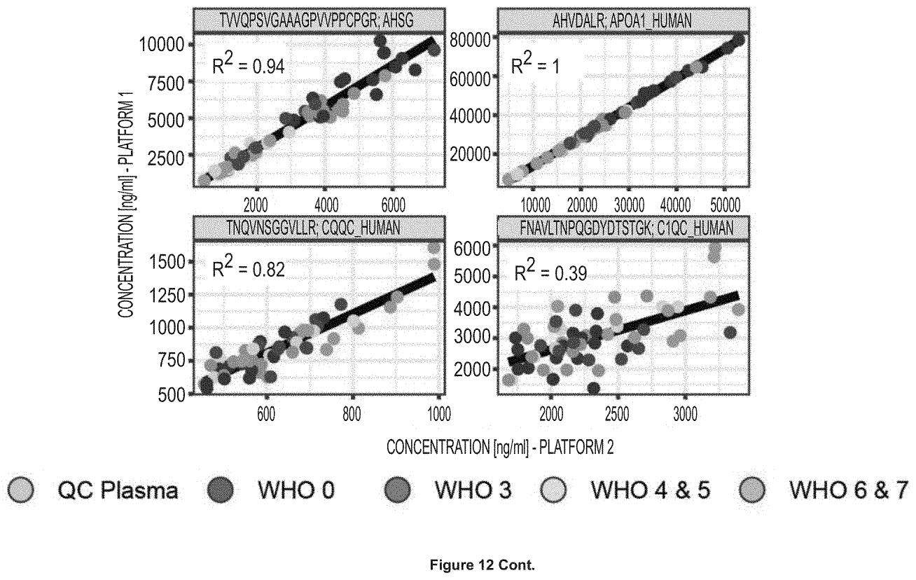

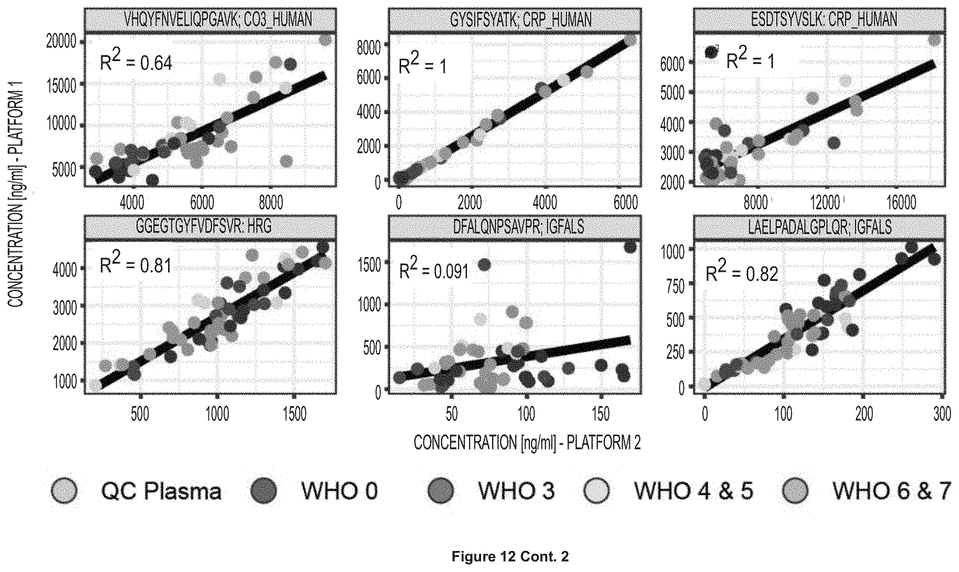

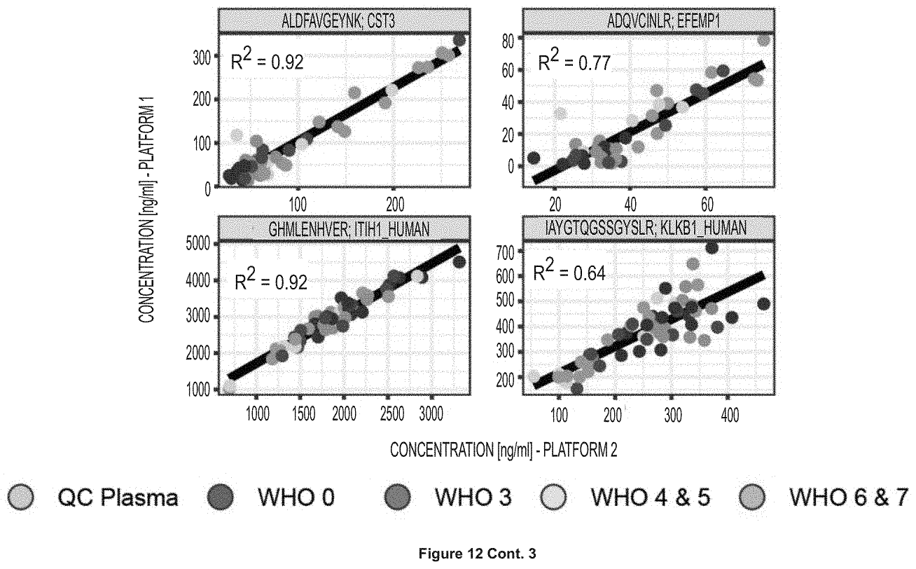

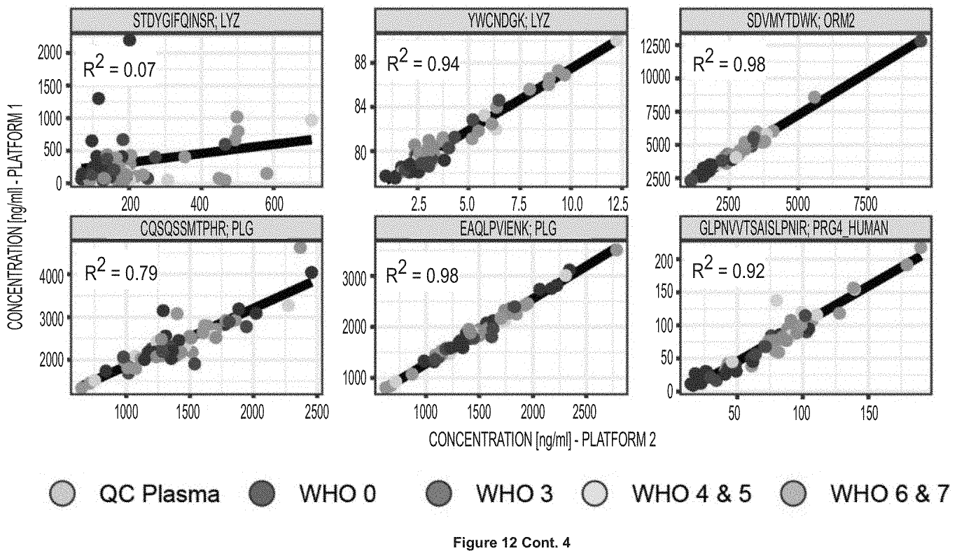

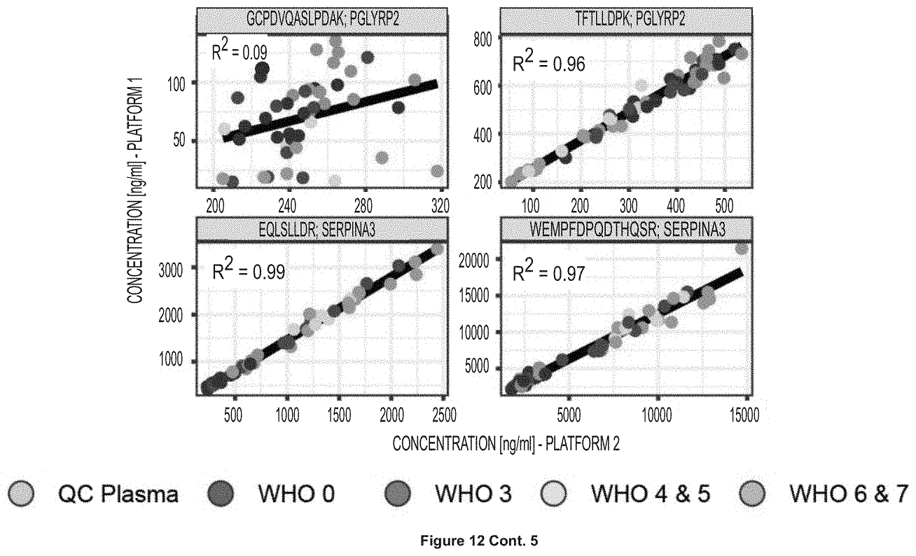

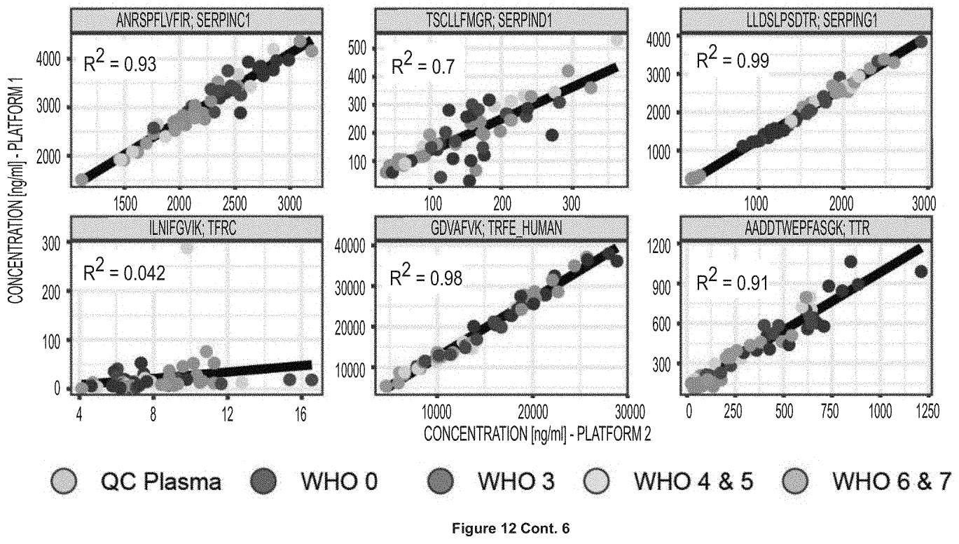

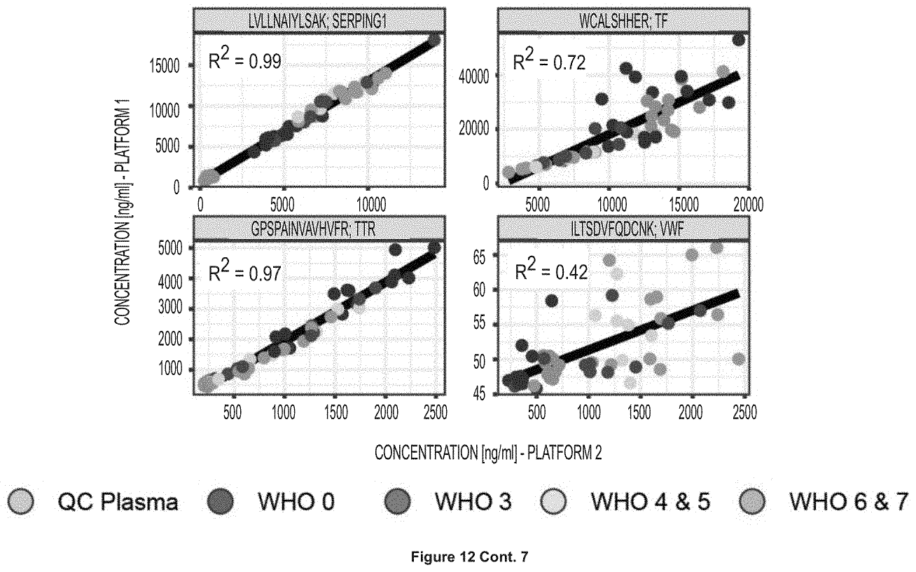

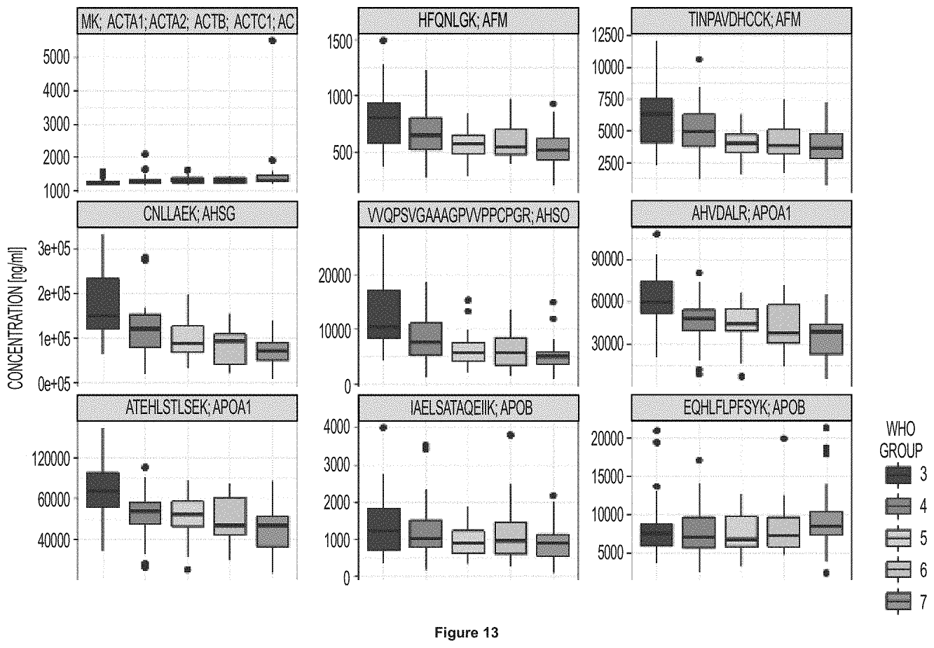

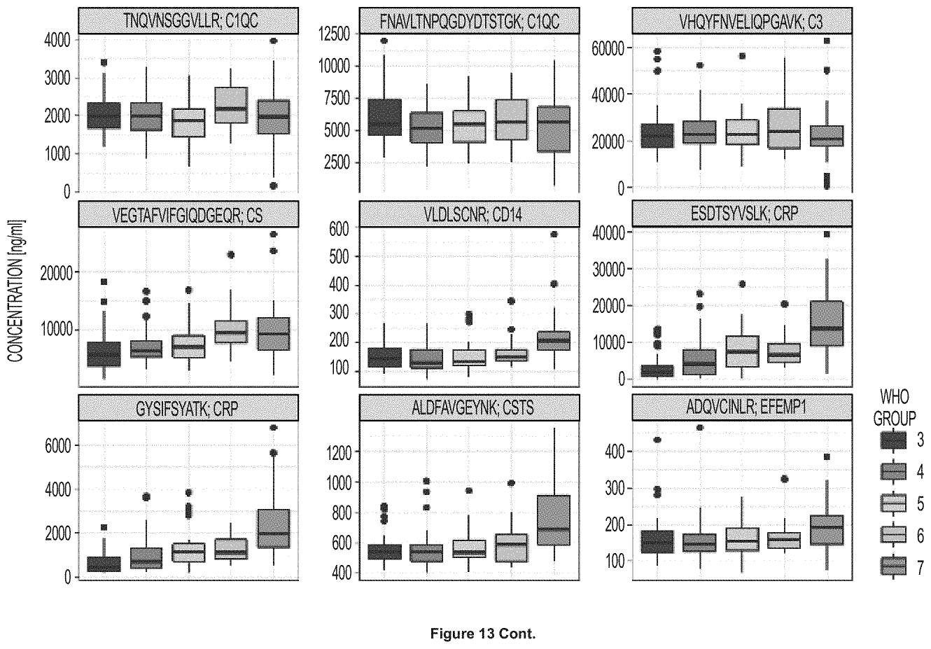

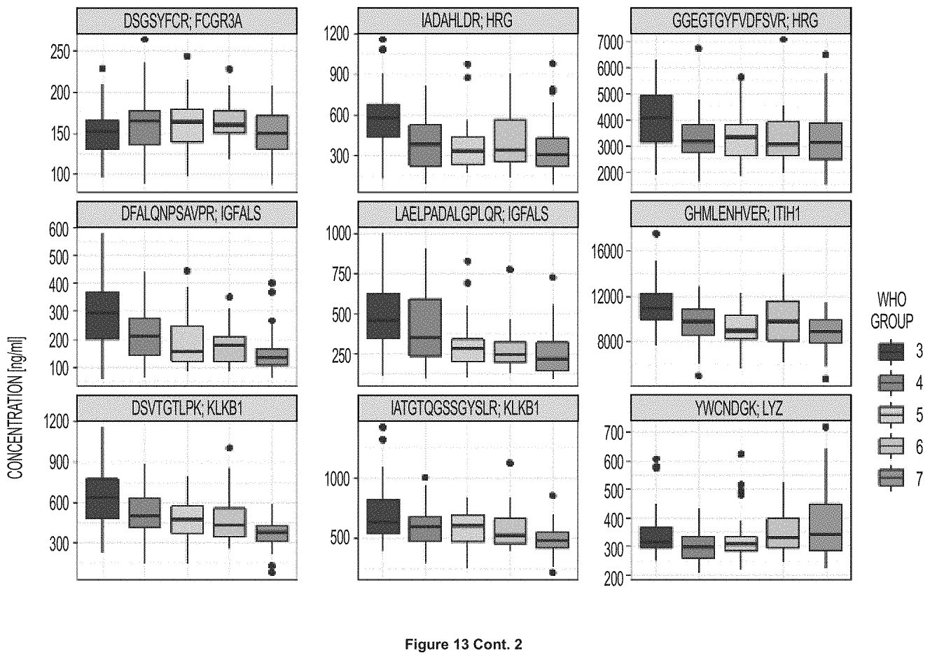

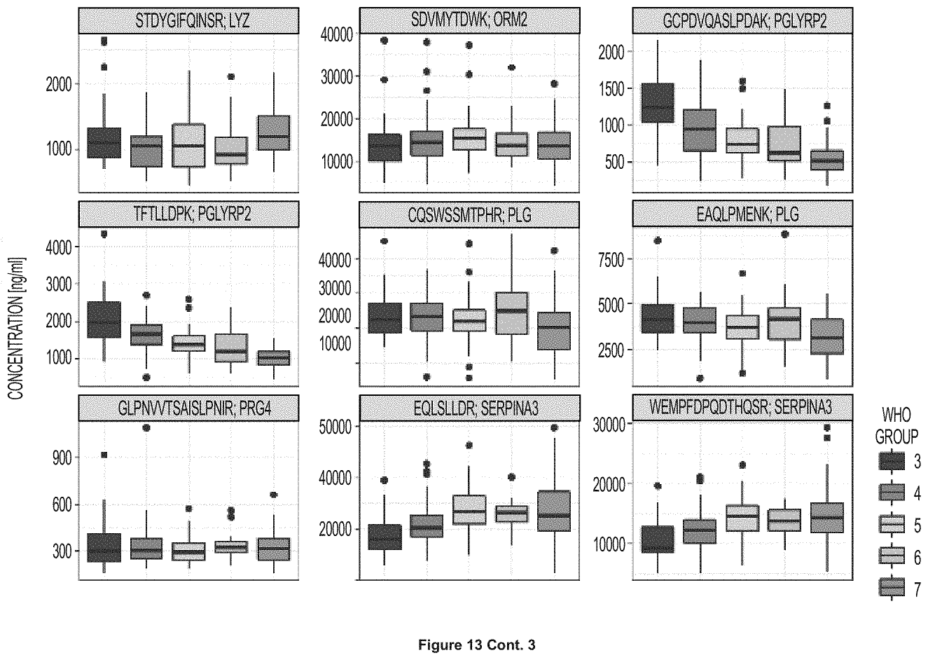

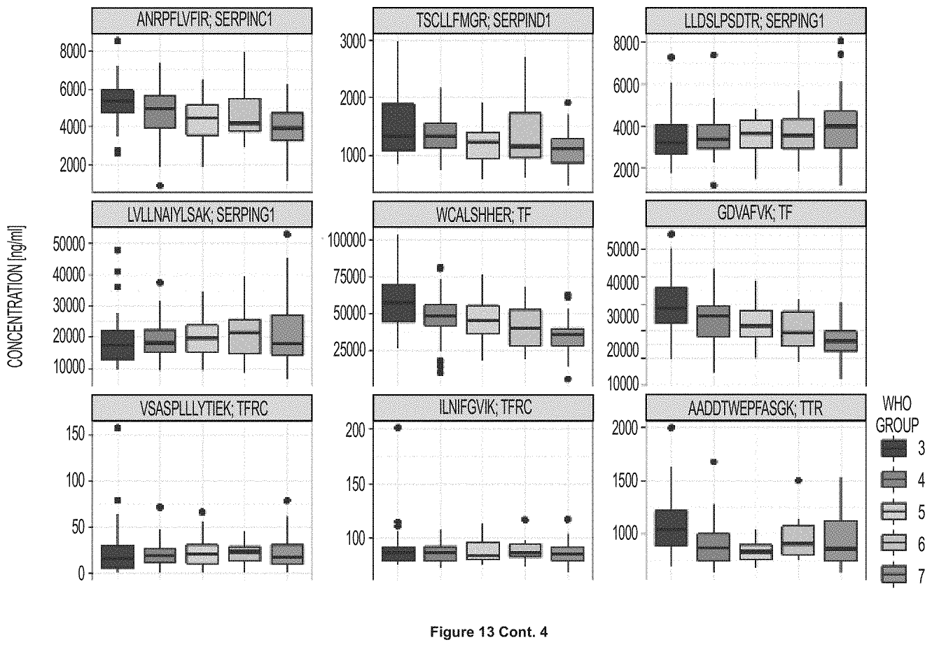

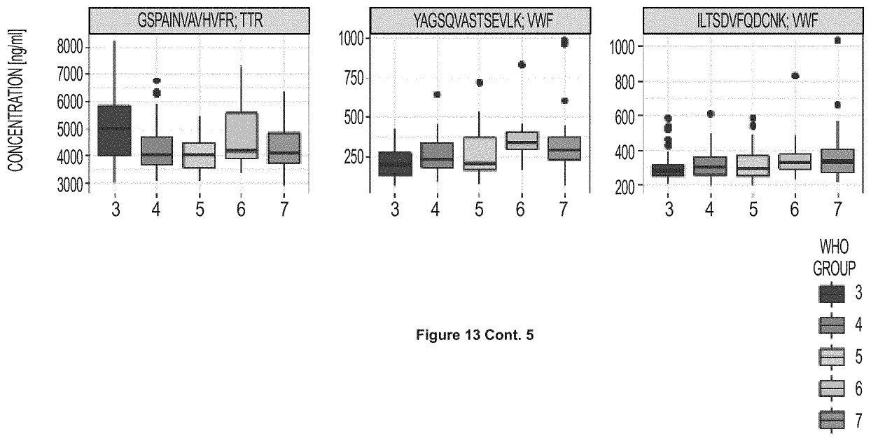

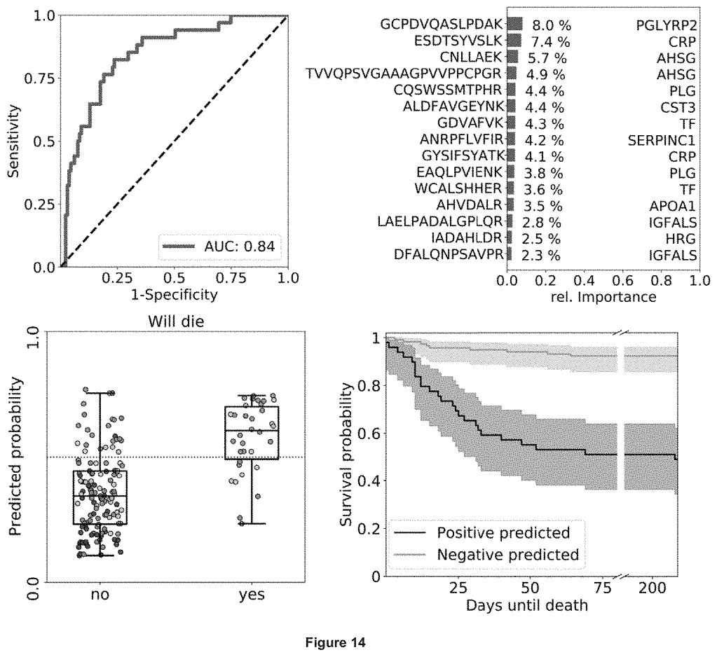

Finally, we examined the capability of the measured peptides for predictions. An outcome predictor (SVM) was trained in a cross-validated manner to differentiate patients who survived COVID-19, from patients who had a fatal disease (First sample taken for each patient, n=165, n_died=34) (FIG. 10). The trained death-predictor was able to correctly classify 82.4% of the patients (sensitivity=0.824, specificity=0.824, AUROC=0.872) that were withheld while training the model. Of note, the three samples with the smallest decision function across patients with fatal outcome did all belong to WHO 4 patients; i.e. corresponds to patients that deteriorated despite presenting with a relatively mild phenotype. Two out of these three false negative predicted cases were kept in hospital for a very long period of 42 and 64 days, respectively, after the sample was taken so that the time between sample and outcome was much longer than for most patients in the training and validation set. The false positive predictions show half of the WHO 7 patients (critical patients with additional organ-support) that will survive the disease. Nonetheless, for the other half the correct negative outcome can be predicted. Across the other WHO grades a good predictability can be observed.

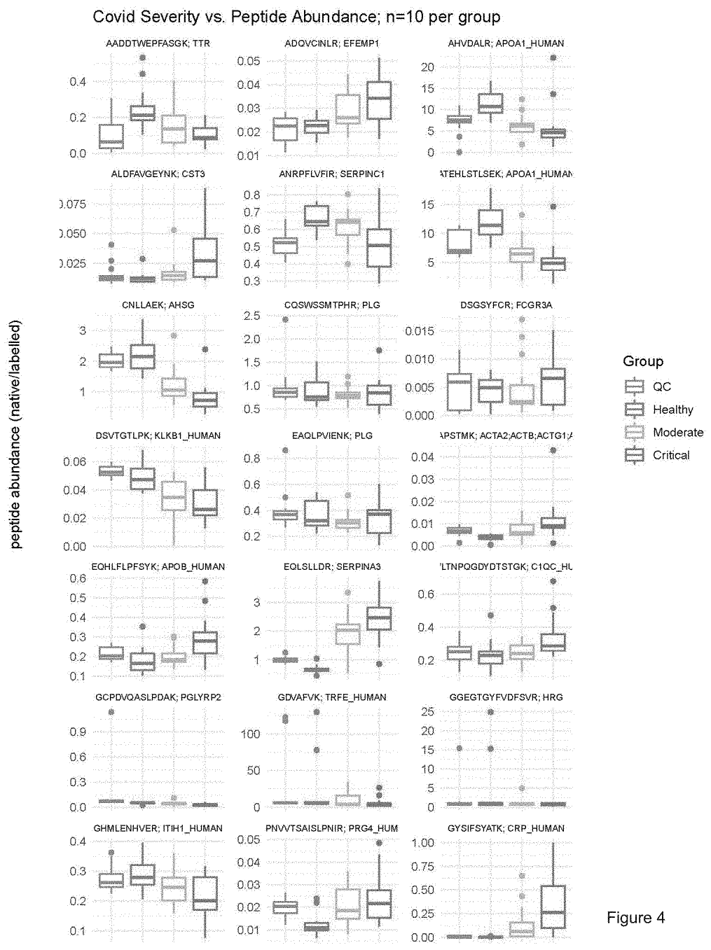

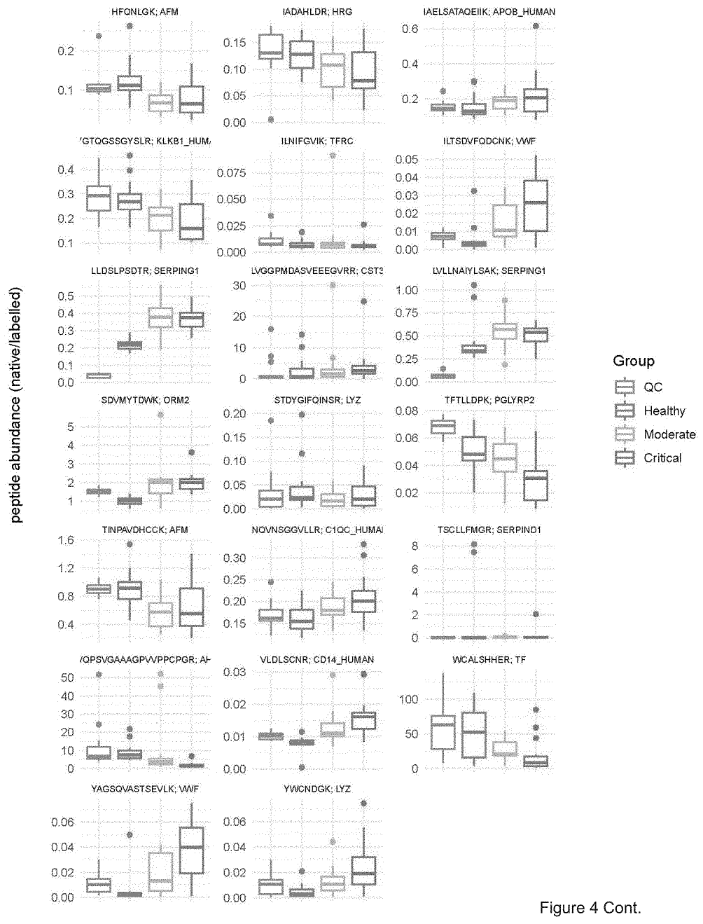

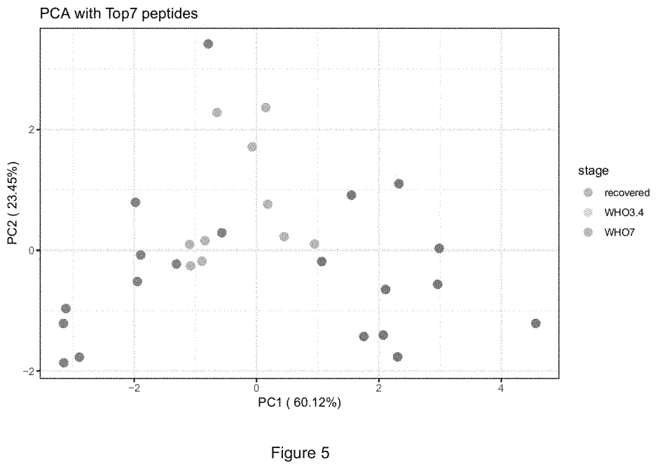

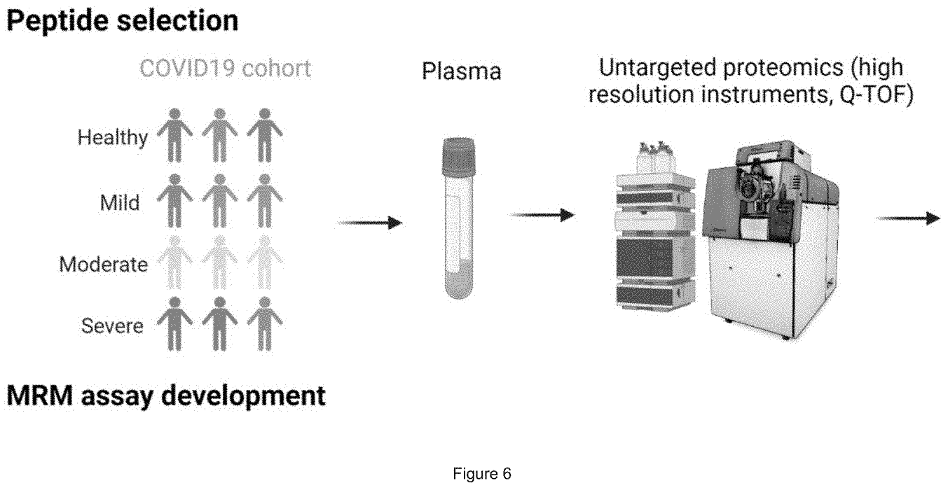

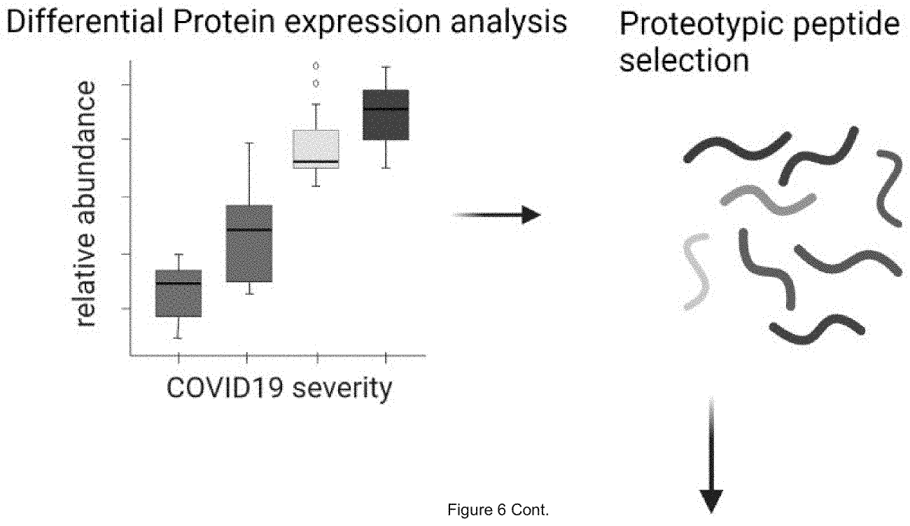

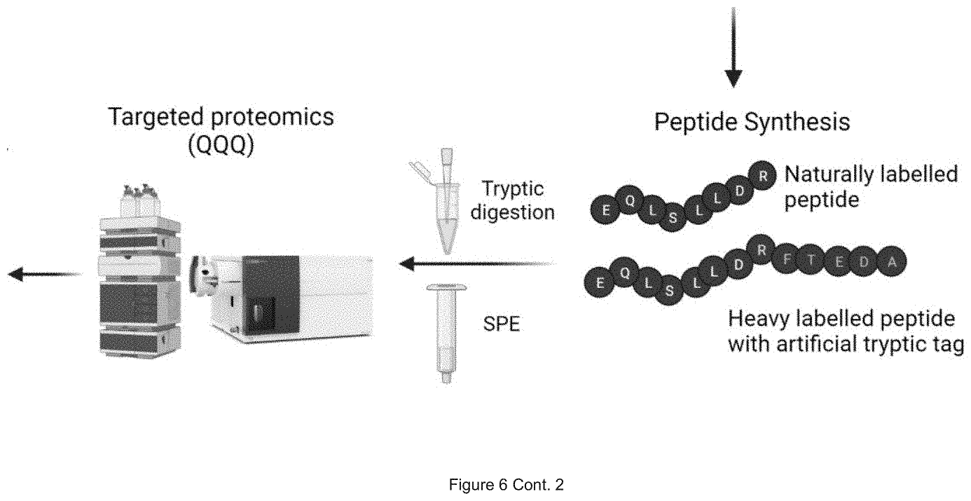

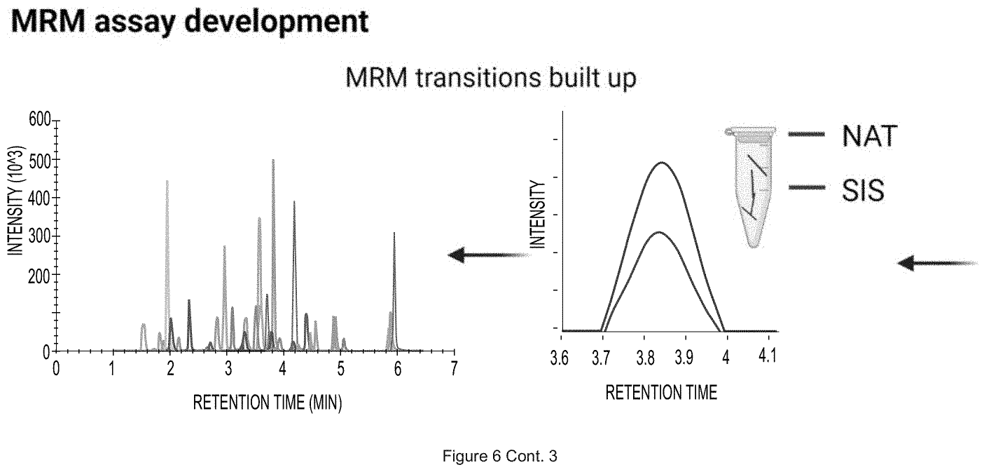

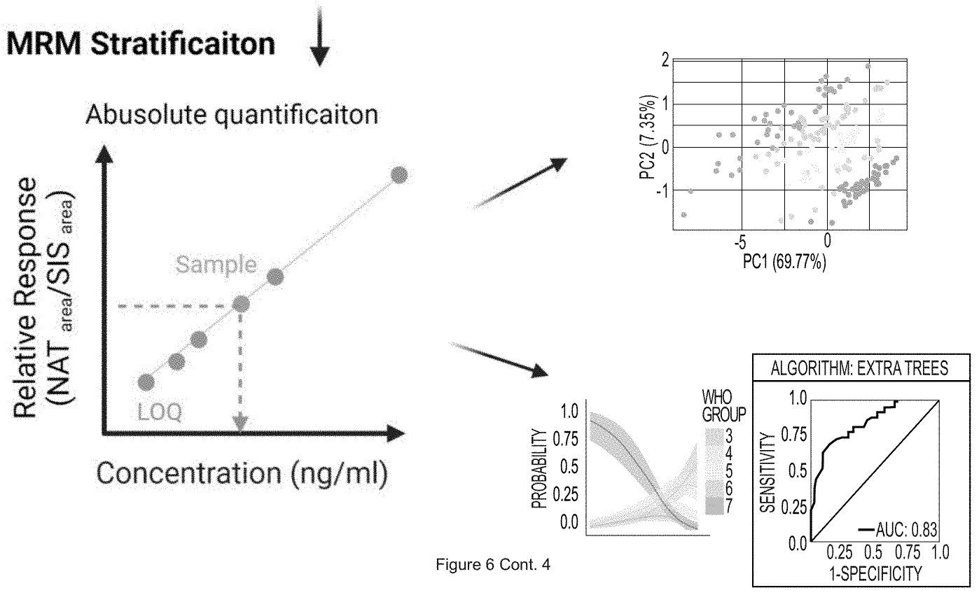

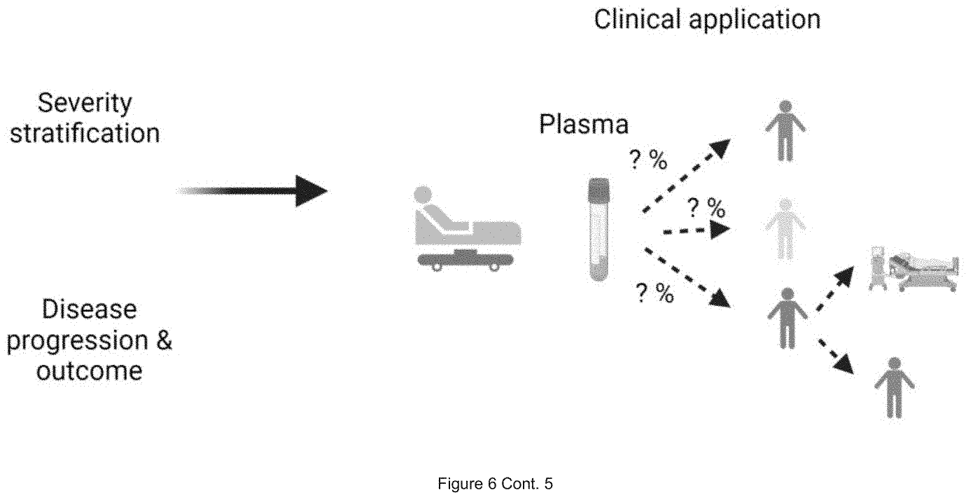

Plotting the predicted outcome with respect to the time until the death (Kaplan-Meier survival analysis, FIG. 10) denotes no clear tendency for the correct and false predictions. To show that the predicting capabilities aren't limited to the method another predictor (extra-trees) using the same setup was evaluated (Supplementary FIG. 4). This predictor, although the metrics are slightly worse (sensitivity=0.735, specificity=0.817, AUROC=0.844), shows a comparable performance to the presented one and enables a ranking of the extracted features. Notably, for several proteins (CRP, AHSG, PLG, TF, IGFALS) more than one peptide was used as an important feature (Top 15 features) by the predictor.

To evaluate how well the predictor performs compared to the clinically established scores, we determined the Sepsis-related Organ Failure Assessment score (SOFA), Acute Physiology And Chronic Health Evaluation (APACHE II) and Charlson Comorbidity Index (CCI), all which are in clinical use. The proteomics predictor significantly outperformed all three clinical scores (AUC of the ROC-curves). The SOFA-score and the APACHE II-score, which are directly linked to the severity of the patient, performed the best among the three “conventional”/established scores tested. For both SOFA and APACHE II scores one can observe an early enrichment of critical cases who will die. However, due to only capturing the current state of the disease, a future prediction especially for non-critical patients can not be performed reliably.

In conclusion, we demonstrate the development of a fast, multiplexed, sensitive LC-MS peptide assay that captures hallmarks of COVID19 disease severity and progression. The assay is easily translatable, sensitive, and specific, and thus well suited for rapid translation into a clinical test. Further, the fast and multiplexed nature of the presented method gives suitable throughput for large patient cohorts or routine diagnostic applications.

Discussion

Novel infectious diseases such as COVID-19 that lack immediate treatment options can quickly challenge health systems on a global scale. Considering the enormous pressure on intensive care units, there is an urgent clinical need for assays that capture and monitor the individual response of patients. Such personalized tests can support an objective clinical decision making that otherwise depends on confounders like age, and could guide development of novel treatments. While a range of such assays and clinical scoring systems have been applied, they often rely on a limited number of biomarkers that do not necessarily capture all major features associated with complex diseases such as COVID-19.

Here, we presented how the combination of high-throughput proteomics for identification of biomarker signatures, followed by the development and validation of a targeted, clinically translatable and scalable LC-MS/MS based assay is a powerful strategy to rapidly transition from a discovery approach to a platform that can be deployed in a clinical setup.

Targeted mass spectrometry using multiple reaction monitoring (MRM) is a method of choice for quantification of multiplex or multiparametric marker panels in biofluids. This is because LC-MRM i) provides excellent sensitivity and specificity, ii) has the possibility to easily include internal standards that give the assay precision and enable control over potential matrix effects (33, 34), iii) facilitates absolute quantification, enabling cross-platform transferability (35, 36). iv) has a large dynamic range (4 orders of magnitude of linear range in the presented assay) (37), which makes it possible to compare biomarkers with large abundance differences within one run, facilitating multiplexing of many biomarkers in parallel; and further, it facilitates sample preparation as matrix depletion is not necessarily required (38). Given the simplicity, flexibility, and multiplexability of LC-MS based MRM assays, the initial panel of peptides can be large, increasing the chance of finding a reliable set of reproducible biomarkers based on hierarchical filtering and selection of the most suitable markers during establishment of the targeted assay (39,40). Finally, although LC-MRM assays require analytical expertise and are labor-intensive to set up, they are highly cost-effective to run. In our study, we also overcome a common limitation of proteomic assays for their routine use—their dependency on low flow rate-chromatography (Bian et al. 2020; Song et al. 2017; Gao et al. 2020). Exploiting the high sensitivity of contemporary triple-quadrupole mass spectrometers, we demonstrate the accurate quantification of the peptide panel using analytical flow rate chromatography, which not only is robust and fast, but also available in typical clinical laboratories, greatly simplifying the application our assay in the routine.

The developed biomarker panel includes 50 proteotypic peptides derived from 30 plasma proteins. The peptides were selected from discovery proteomic data (13, 22, 23, 28) for their ability to predict the patient stay in hospital and to indicate the likelihood of future worsening, and were found to change in abundance depending on the treatment escalation level according to the WHO ordinal scale, introduced as of April 2020 (41), used as a measure of COVID19 disease severity. These proteins were all found to belong to biological processes important for the COVID19 host response, like the coagulation system, the complement cascade or metabolism. The assay is hence monitoring processes causal to the disease progression. In this study, we established the assay on two routine-laboratory compatible LC-MRM platforms, and performed an elaborate analytical validation procedure of the technical aspects of the assay. We demonstrate excellent reproducibility across two different mass spectrometric technologies, and reveal for each peptide the optimal measurement parameters, dynamic range, as well as sensitivity to matrix effects. As such, the assay could easily be translated into a multiplexed, high-throughput clinical assay that captures hallmarks of the COVID-19 pathophysiology.

We confirm in two independent patient cohorts, that the panel assay captures the severity of the COVID19 affected individual, and is discriminatory about the treatment levels. Moreover, the assay is prognostic about the outcome for the most severely affected COVID19 patients. Thus, the panel assay parameters could be used to assess the current state of the patient, help to monitor efficacy of novel treatments, or stratify patients based on their responsiveness to novel clinical interventions for COVID-19 therapies. Furthermore, the assay can be employed to predict the progression of COVID-19 disease, as exemplified by the performed prediction of disease outcome weeks into the future. Such knowledge can help guide clinical decision making, and optimise hospital resource planning, specifically in critical situations of the pandemic that threatens the capacities of hospitals.
Proteomics can be prognostic for outcome in patients with similar disease severity, e.g. among severely affected patients, that are difficult to distinguish by clinical parameters. This means that the prognosis of survival using targeted proteomics could be improved beyond what was shown in this study for ‘within-severity-group’ prognosis, if biomarker panels specifically for stratification within the respective COVID-19 severity group would be selected. Indeed, particularly within the group of severely affected individuals, some patients were predicted incorrectly, i.e. survived despite being predicted as non-survivors, or vice versa. We assessed on a patient-by-patient basis whether there are medical reasons that could explain wrong predictions. Plotting outcome with respect to the time until death denotes no clear tendency for the correct and false predictions. However, we noted that the three samples with the smallest decision function across wrongly predicted patients with fatal outcomes belonged to WHO 4 patients that had DNI (‘do not intubate’) orders in place. It is hence plausible that the assay correctly identified a milder form of COVID-19 in these three individuals; i.e. that without a strong comorbidity or a DNI order in place, these might have had a good chance to survive COVID-19. In a similar fashion, we recently reported two cases where the proteomic signatures of patients correctly distinguished an Influenza B from a COVID-19 infection, and that highlighted a patient that had to undergo chemotherapeutic cancer treatment just days before a COVID-19 infection. Thus, protein signatures could in principle distinguish different (respiratory) infections or comorbidities, while additional research will be required to establish this for the presented protein panel. COVID-19 will remain a central public health issue for the foreseeable future as new variants of concern with capacity to evade vaccine induced immunity continue to emerge. The underpinning targeted proteomics platform supports rapid iteration of the panel composition in case additional prognostic biomarkers are discovered. Taken together, this peptide panel and the underlying analytical platform, holds potential to support a broader, continuous pandemic response in addition to its utility in hospitalised patient cohorts which we demonstrate in the present study

Methods

Patient Cohorts

Patient samples were collected as described previously (13, 22, 23, 28) as part of a prospective observational cohort study Pa-COVID-19 at Charité—Universitätsmedizin Berlin. The study protocol has been described in detail before (Kurth et al., 2020). The study is registered in the German and the WHO international registry for clinical studies (DRKS00021688).

Reagents and Peptide Standards

Synthetic reference peptides were from Pepmic (Suzhou, China). Native peptides were synthesised at ≥95% purity and internal standard peptides—at ≥70% purity. Internal standards contained 4-6 amino acid tryptic tags mimicking the sequence in a corresponding human plasma protein and were labelled on C-terminal lysine (K) or arginine (R) amino acids with stable isotopes (K(U-13C6,15N2) or R(U-13C6,15N4)). Water was from Merck (LiChrosolv LC-MS grade; Cat #115333), acetonitrile was from Biosolve (LC-MS grade; Cat #012078), trypsin (Sequence grade; Cat #V511X) was from Promega, 1,4-Dithiothreitol (DTT; Cat #6908.2) from Carl-Roth, iodoacetamide (IAA; Bioultra; Cat #11149) and urea (puriss. P.a., reag. Ph. Eur.; Cat #33247) were from Sigma-Aldrich, ammonium bicarbonate (Eluent additive for LC-MS; Cat #40867) and Dimethyl sulfoxide (DMSO; Cat #41648) were from Fluka, formic acid (LC-MS Grade; Eluent additive for LC-MS; Cat #85178) was from Thermo Scientific™, bovine serum albumin (BSA) (Albumin Bovine Fraction V, Very Low Endotoxin, Fatty Acid-free; Cat #47299) was from Serva, commercial human plasma samples (Human Source Plasma, LOT #20CILP1034) was from zenbio.

All peptide stock solutions were prepared at 1 mg/ml concentration in 50:50 ddH2O: acetonitrile mix, except for STDYGIFQINSR (SEQ ID No: 22) and VEGTAFVIFGIQDGEQR (SEQ ID No: 51) where 200 μl of DMSO were added to solubilise the peptides at 5 mg/ml which were then diluted to small aliquot of 1 mg/ml with 50:50 ddH2O: acetonitrile mix before each sample preparation). Internal standard mix was prepared by pooling 20 μl of each heavy isotope-labeled peptide, evaporating 200 μl of this mix to dryness and reconstituting in a denaturation buffer to the final concentration of 1.4 μg/ml for each peptide. Cassetted calibration curves were prepared by serial dilution of pooled native reference peptide standards as described in Analytical method validation. After serial dilution, these samples were treated identically to respective clinical samples.

Peptide Selection

Peptides were selected based on discovery LC-MS/MS data from our prior studies (Messner et al., 2020; Demichev et al., 2021), where we identified a protein biomarker signature predictive of COVID-19 outcome. We selected a total of 50 proteotypic reference peptides from 30 human plasma proteins (1-2 peptides per protein) that had the highest predictive power in previous studies. The predictive performance in the following statistical tests was used for selection: (i) prediction of the remaining time in hospital for mild patients, (ii) prediction of future worsening for patients of all severity grades and (iii) stratification of patients of different severity grades. For each protein, only peptides that showed a good signal in the SWATH data as well as were predicted to be suitable for MRM by the Peptide Analyzing Tool (Thermo) were selected. Each native reference peptide was unique to a corresponding protein within the human proteome in a Uniprot BLAST search. Isotope-labelled internal standards were designed based on the selected native reference peptides.

Sample Preparation

Samples were prepared as described previously (Messner et al., 2020) with minor modifications. Briefly, clinical samples and calibration lines in Cohort 1 were prepared as follows: 5 μl of plasma or serum sample were added to 55 μl of denaturation buffer, composed of 5 μl 8M Urea, 100 mM ammonium bicarbonate, 5 μl 50 mM dithiothreitol (DTT) and peptide internal standard mix. The samples were incubated for 1 h at room temperature before addition of 5 μl of 100 mM iodoacetamide (IAA). After a 30 min incubation at room temperature the samples were diluted with 340 μl of 100 mM ammonium bicarbonate and digested overnight with 23 μl of 0.1 μg/μl trypsin at 37° C. The digestion was quenched by adding 50 μl of 10% v/v formic acid. The resulting tryptic peptides were purified on a 96-well C18-based solid phase extraction (SPE) plate (BioPureSPE Macro 96-well, 100 mg PROTO C18, The Nest Group). The purified samples were resuspended in 120 μl of 0.1% formic acid and 20 μl were injected into the LC-MS/MS system.

Samples in cohort 2 were prepared as described above, with one modification. As these samples had already been prepared for a discovery proteomics study, internal standards were digested separately and added to pre-digested clinical and calibration line samples before their injection into the LC-MS/MS system. Quality control (QC) samples consisted of pooled commercial control and COVID-19 human plasma samples (as described in a previous publication (Messner et al. 2020)), and were prepared alongside clinical and calibration curve samples in each cohort.

The COVID-19 sample pools used for the analytical validation were generated by pooling 5 μl of patient plasma from cohort 2 according to their WHO treatment severity score. Only samples of patients that had not received dexamethasone were used.

Liquid Chromatography-Tandem Mass Spectrometry

Tryptic peptides were quantified on 2 liquid chromatography-triple quadrupole mass spectrometry (LC-MS/MS) platforms—7500 (Sciex) and 6495c (Agilent).

6495c (Agilent) LC-MS/MS Method

All clinical samples were analysed on the Agilent 6495c mass spectrometer, coupled to Agilent 1290 Infinity UHPLC system. Prior to MS analysis, samples were chromatographically separated on Agilent InfinityLab Poroshell 120 EC-C18 1.9 μm, 2.1×50 mm column heated to 45° C. and with a flow rate of 800 μl/min. Linear gradients employed were as follows (time, % of mobile phase B): 0 min, 3%; 1 min, 3%; 7.5 min, 35%; 8 min 98%; 8.5 min, 98%; 8.6 min, 3%; 10 min, 3% where mobile phase A & B are 0.1% formic acid in water and 0.1% formic acid in acetonitrile respectively.

The 6495c mass spectrometer was controlled by Agilent's MassHunter Workstation software (LC-MS/MS Data Acquisition for 6400 series Triple Quadrupole, Version 10.1) and was operated in positive electrospray ionisation mode with the following parameters: 3500 V capillary voltage (positive), 0 V nozzle voltage (positive), 12 L/min sheath gas flow at a temperature of 280° C., 17 L/min gas flow at a temperature of 170° C., 40 psi nebulizer gas flow, 166 V default fragmentor voltage, 5 V default cell accelerator potential. Samples were analysed in dynamic MRM mode with both quadrupoles operated in unit resolution. All other MRM parameters, including monitored transitions and scheduling are provided in the Table 6.

7500 (Sciex) LC-MS/MS Method

Samples from the Kubler cohort (cohort 2) were analysed on a SCIEX 7500 mass spectrometer coupled to an ExionLC AD UHPLC system (SCIEX, UK) in addition to the analysis on the Agilent platform. Prior to MS analysis, samples were chromatographically separated on a Phenomenex Luna Omega Polar 3 μm, 100×2.1 mm column heated to 40° C. and with a flow rate of 500 μl/min. Linear gradients employed were as follows (time, % of mobile phase B): 0 min, 3%; 1 min, 3%; 7.5 min, 30%; 8 min 95%; 8.5 min, 95%; 8.6 min, 3%; 10 min, 3% where mobile phase A & B are 0.1% formic acid in water and 0.1% formic acid in acetonitrile respectively.

The 7500 triple quadrupole mass spectrometer was operated in positive electrospray ionisation mode with the following ion source parameters: 1750 V Ionspray voltage, 40 psi curtain gas, 40 psi Ion source gas 1, 70 psi ion source gas 2 and 500° C. temperature. Samples were analysed in MRM mode with both quadrupoles operated in unit resolution. All other MRM parameters, including monitored transitions and scheduling are provided in the Table 7.

Establishment of the MRM Based Assay

The assay was first set up on the 6495C (Agilent) system. Preliminary transitions for the 50 selected putative biomarkers (consisting of several precursor ion charge states and respective product ions) were predicted by Skyline v21.1.0.146. The native peptide standard solution was then infused into LC-MS/MS system and 1 precursor ion per peptide with the highest relative intensity and 5 most abundant product ions were selected for collision energy optimisation as provided by Skyline. From these 5 product ions, 2-5 experimentally optimised ion transitions per native peptide were ultimately selected in the panel based on the following criteria: i) highest relative signal intensity, ii) optimal chromatographic peak shape and iii) absence of interfering signals. Product ions of <300 m/z were excluded where possible to ensure specificity. Precursor and product ion-matched ISTD transitions were also included. Lastly, all selected transitions were combined into one scheduled MRM method, which was subsequently analytically validated. In the designed method, the most abundant transition for each peptide was used for quantification and 1-4 less abundant transitions were used as qualifiers (data not shown). For analytical cross-platform and cross-laboratory validation, the assay was set up on the 7500 (SCIEX) system in parallel following that approach (data not shown).

Mass Spectrometry Data Processing

Mass spectrometry data processing was performed with vendor-specific software: MassHunter Quantitative Analysis, v0.1, Agilent Technologies and SCIEX OS Software v2.0.1. Peak selection and integration were manually assessed before exporting the peak area values to .csv for further analysis. Peptide absolute concentration was determined from calibration curves, constructed with native and heavy isotope-labelled synthetic reference standards. Of note, SIL internal standards for 5 corresponding native peptides could not be detected on the 6495C system. To quantify these native peptides, we used other, closely eluting SIL internal standards in the assay: AADDTWEPFASGK(U-¹³C₆,¹⁵N₂ (SEQ ID No: 87) was used for ASDTAMYYCAR (SEQ ID No. 50), GYSIFSYATK(U-¹³C₆,¹⁵N₂) (SEQ ID No: 73) for GSPAINVAVHVFR (SEQ ID No: 34) and WEMPFDPQDTHQSR (SEQ ID No: 11), ANRPFLVFIR(U-¹³C₆,¹⁵N₄) (SEQ ID No: 90) for LAELPADALGPLQR (SEQ ID No: 49) and VSASPLLYTLIEK(U-¹³C₆,¹⁵N₂) (SEQ ID No: 97) for VEGTAFVIFGIQDGEQR (SEQ ID No: 51). In addition, due to low signal intensity of pre-assigned quantifier transitions (transitions with matched precursor and product ions across native and SIL peptides), we chose other transitions with higher signal intensity for 4 SIL peptides, even if they did not match the fragmentation pattern of their respective native peptides. The transitions used for quantification on both 6495C and 7500 LC-MS/MS platforms are shown in Tables 6 and 7 respectively. Linear regression analysis of each calibration curve was performed in Rstudio or Sciex OS (with 1/x weighting) and the respective peptide concentration in patient samples was expressed in ng/ml.

Analytical Method Validation

Method analytical validation was performed based on FDA Bioanalytical Method Validation criteria (42) where sensitivity, specificity, intra, inter-repeatability, accuracy and matrix effects have been assessed. A total of 5 independent calibration curves were prepared by serial dilution of native reference peptide standards in assay buffer (1), surrogate matrix (3) and pooled human plasma (1) across the final sample peptide concentration range of 0-1.63 μg/ml. Surrogate matrix (40 mg/ml BSA) calibration curves were prepared and analysed across 3 separate batches and all calibration curve samples were analysed in quintuplets. Linear 1/x weighted calibration curves were obtained for all native peptide reference standards in order to check the linearity of the response. Lower limit of quantification (LLOQ) was defined as the lowest calibration sample on the linear curve with a CV 20%. Ten peptides where low endogenous concentrations were observed in a pooled clinical subset of samples, LLOQ criteria were expanded to CV≤40% to prevent missing values where these peptides that were highly differentially expressed between COVID-19 severity groups were successfully quantified. Upper limit of quantification (ULOQ) was defined as the highest calibration sample on the linear curve with a CV≤20%. Accuracy was assessed by treating 1 of the 5 replicates in each calibration curve in the surrogate matrix as pseudo-unknown samples and quantifying with the curve generated from the remaining 4 replicates. The final accuracy was determined as the median of all calculated accuracy of each peptide. Matrix effects were measured by comparing the slopes of curves from calibration samples prepared in a BSA matrix and pooled human plasma. Here an Extra Sum of Square F test was used for statistical comparison with a p-value<0.05 indicating potential matrix effects.

Data Analysis and Visualisation, Statistics

Significance testing of the trend between absolute peptide concentrations and the ordinal classification as provided by the WHO treatment escalation scale (levels as indicated) was performed using Kendall's tau (KT) statistics as implemented in the “EnvStats v2.4.0” R package “kendallTrendTest” function. For cohort 1 the KT statistics was calculated as the trend of peptide quantities against the following WHO groups: 0, 3, 4, 5, 6, 7; for cohort 2, peptide quantities were tested against the following WHO groups: 3, 4, 5, 6, 7. A full summary of statistical test results is provided in Table 9. Multiple testing correction was performed by controlling for false discovery rate using the Benjamini-Hochberg procedure (43) as provided by the R package “stats v4.1.0”-“p.adjust” function. Principal component analysis was performed using the R function “prcomp” from the “stats 4.1.0” package and visualized using “ggplot2 v3.3.5”.

Prediction of WHO Grade and Disease Outcome

Clinical scores (CCI [39], SOFA [40], APACHE II [41], and ABCS [22]) were extracted from the clinical information system or, where missing, manually calculated. CCI and APACHE II were determined at time of admission, while SOFA was calculated for time of sampling. ABCS was calculated for admission and time of sampling. For ABCS, up to two missing laboratory values (either lymphocytes, blood urea nitrogen (BUN) or aspartate aminotransferase (ASAT)) were imputed by using the median value of patients within the same maximum WHO severity group. Note that due to imputation of the ABCS score memory leakage between training and test data for the ABCS score models can not be excluded. For the prediction of the current WHO grade and for the outcome prediction a Support Vector Machine was used as implemented in scikit-learn 0.23.2 (sklearn.svm.SVC) (44) using default parameters (rbf-kernel) and balanced class weights (class_weight=“balanced”). For one peptide (VSASPLLYTLIEK (SEQ ID No: 45)) negative values were present after external calibration. Those values were replaced by the minimal positive value of the respective peptide measured over all samples. Two peptides were removed (ASDTAMYYCAR (SEQ ID No: 50) and LVGGPMDASVEEEGVRR (SEQ ID No: 9)) as they were not reliably quantified leaving 48 peptides for the analysis. For every patient the first sample measured was selected (n=165). All patients with unknown WHO grade/outcome were neglected. All data were log 2-transformed and scaled to 0 mean and 1 variance fitted on the training data (sklearn.preprocessing.StandardScaler). The model was trained and validated using a shuffled stratified 5-fold cross-validation (sklearn.model_selection.StratifiedKFold) to assure that every split has a comparable case-to-control ratio and that every sample was used in 4 runs for training and in the remaining run for validating the trained model not including this sample. For reproducibility the seed was fixed to 42. For models trained on severity scores, only samples for which the respective score was determined were included in model construction and testing.

Decision function, ROC-Curve, accuracy, sensitivity and specificity were calculated using scikit-learn 0.23.2. For the Kaplan-Meier estimate lifelines 0.26.0 (45) was used. The data were divided in positive and negative predicted cases. For the true positive and false negative predicted cases, the days until death were included in the model. The samples for people who left the hospital alive were censored. Samples with missing time until outcome for patients who died were neglected. The confidence intervals were calculated using Greenwood's Exponential formula as implemented in lifelines 0.26.0 (alpha=0.05).

In addition, a predictor based on the extra-trees algorithm implemented in scikit-learn 0.23.2 (sklearn.ensemble.ExtraTreesClassifier) was evaluated. The same approach as described above was applied with the differences that the data weren't log 2-transformed and scaled as this isn't needed for a tree-based classifier. In addition, the maximal depth of the trees was set to 3 (max depth=3) to compensate for overfitting issues due to limited data set size.

### REFERENCES

1. Dong E, Du H, Gardner L. An interactive web-based dashboard to track COVID-19 in real time. Lancet Infect Dis. 2020; 20:533-4.
2. Pouwels K B, Pritchard E, Matthews P C, Stoesser N, Eyre D W, Vihta K-D, et al. Effect of Delta variant on viral burden and vaccine effectiveness against new SARS-CoV-2 infections in the U K. Nat Med [Internet]. 2021; Available from: http://dx.doi.org/10.1038/s41591-021-01548-7
3. Kearns P, Siebert S, Willicombe M, Gaskell C, Kirkham A, Pirrie S, et al. Examining the Immunological Effects of COVID-19 Vaccination in Patients with Conditions Potentially Leading to Diminished Immune Response Capacity—The OCTAVE Trial [Internet]. 2021 [cited 2021 Nov. 16]. Available from: https://papers.ssrn.com/abstract=3910058
4. Chemaitelly H, Tang P, Hasan M R, AlMukdad S, Yassine H M, Benslimane F M, et al. Waning of BNT162b2 Vaccine Protection against SARS-CoV-2 Infection in Qatar. N Engl J Med [Internet]. 2021; Available from: http://dx.doi.org/10.1056/NEJMoa2114114
5. Levin E G, Lustig Y, Cohen C, Fluss R, Indenbaum V, Amit S, et al. Waning Immune Humoral Response to BNT162b2 Covid-19 Vaccine over 6 Months. N Engl J Med [Internet]. 2021; Available from: http://dx.doi.org/10.1056/NEJMoa2114583
6. Bian L, Gao F, Zhang J, He Q, Mao Q, Xu M, et al. Effects of SARS-CoV-2 variants on vaccine efficacy and response strategies. Expert Rev Vaccines. 2021; 20:365-73.
7. Twohig K A, Nyberg T, Zaidi A, Thelwall S, Sinnathamby M A, Aliabadi S, et al. Hospital admission and emergency care attendance risk for SARS-CoV-2 delta (B.1.617.2) compared with alpha (B.1.1.7) variants of concern: a cohort study. Lancet Infect Dis [Internet]. 2021; Available from: http://dx.doi.org/10.1016/S1473-3099(21)00475-8
8. Tao K, Tzou P L, Nouhin J, Gupta R K, de Oliveira T, Kosakovsky Pond S L, et al. The biological and clinical significance of emerging SARS-CoV-2 variants. Nat Rev Genet [Internet]. 2021; Available from: http://dx.doi.org/10.1038/s41576-021-00408-x
9. Overmyer K A, Shishkova E, Miller I J, Balnis J, Bernstein M N, Peters-Clarke™, et al. Large-Scale Multi-omic Analysis of COVID-19 Severity. Cell Syst. 2021; 12:23-40.e7.
10. D'Alessandro A, Thomas T, Dzieciatkowska M, Hill R C, Francis R O, Hudson K E, et al. Serum Proteomics in COVID-19 Patients: Altered Coagulation and Complement Status as a Function of IL-6 Level. J Proteome Res. 2020; 19:4417-27.
11. Vllmy F, van den Toorn H, Zenezini Chiozzi R, Zucchetti O, Papi A, Volta C A, et al. A serum proteome signature to predict mortality in severe COVID-19 patients. Life Sci Alliance [Internet]. 2021; 4. Available from: http://dx.doi.org/10.26508/lsa.202101099
12. Couzin-Frankel J. The mystery of the pandemic's “happy hypoxia.” Science. 2020; 368:455-6.
13. Demichev V, Tober-Lau P, Lemke O, Nazarenko T, Thibeault C, Whitwell H, et al. A time-resolved proteomic and prognostic map of COVID-19. Cell Syst. 2021; 12:780-94.e7.
14. Vegvari C, Caust E, Hadjichrysanthou C, Lawrence E, Weverling G-J, de Wolf F, et al. Using Clinical Trial Simulators to Analyse the Sources of Variance in Clinical Trials of Novel Therapies for Acute Viral Infections. PLoS One. 2016; 11:e0156622.
15. Shen C, Ferro E G, Xu H, Kramer D B, Patell R, Kazi D S. Underperformance of Contemporary Phase III Oncology Trials and Strategies for Improvement. J Natl Compr Canc Netw. 2021; 19:1072-8.
16. Gutmann C, Takov K, Burnap S A, Singh B, Ali H, Theofilatos K, et al. SARS-CoV-2 RNAemia and proteomic trajectories inform prognostication in COVID-19 patients admitted to intensive care. Nat Commun. 2021; 12:3406.
17. Wynants L, Van Calster B, Collins G S, Riley R D, Heinze G, Schuit E, et al. Prediction models for diagnosis and prognosis of covid-19: systematic review and critical appraisal. BMJ. 2020; 369:m1328.
18. Gupta R K, Marks M, Samuels T H A, Luintel A, Rampling T, Chowdhury H, et al. Systematic evaluation and external validation of 22 prognostic models among hospitalised adults with COVID-19: an observational cohort study. Eur Respir J [Internet]. 2020; 56. Available from: http://dx.doi.org/10.1183/13993003.03498-2020
19. Park J, Kim H, Kim S Y, Kim Y, Lee J-S, Dan K, et al. In-depth blood proteome profiling analysis revealed distinct functional characteristics of plasma proteins between severe and non-severe COVID-19 patients. Sci Rep. 2020; 10:22418.
20. Ignjatovic V, Geyer P E, Palaniappan K K, Chaaban J E, Omenn G S, Baker M S, et al. Mass Spectrometry-Based Plasma Proteomics: Considerations from Sample Collection to Achieving Translational Data. J Proteome Res. 2019; 18:4085-97.
21. Filbin M R, Mehta A, Schneider A M, Kays K R, Guess J R, Gentili M, et al. Plasma proteomics reveals tissue-specific cell death and mediators of cell-cell interactions in severe COVID-19 patients. bioRxiv [Internet]. 2020; Available from: http://dx.doi.org/10.1101/2020.11.02.365536
22. Messner C B, Demichev V, Wendisch D, Michalick L, White M, Freiwald A, et al. Ultra-high-throughput clinical proteomics reveals classifiers of COVID-19 infection. Cell Systems [Internet]. 2020; Available from: http://www.sciencedirect.com/science/article/pii/S2405471220301976
23. Messner C B, Demichev V, Bloomfield N, Yu J S L, White M, Kreidl M, et al. Ultra-fast proteomics with Scanning SWATH. Nat Biotechnol [Internet]. 2021; Available from: http://dx.doi.org/10.1038/s41587-021-00860-4
24. Keevil B G. LC-MS/MS analysis of steroids in the clinical laboratory. Clin Biochem. 2016; 49:989-97.
25. Gaudl A, Kratzsch J, Ceglarek U. Advancement in steroid hormone analysis by LC-MS/MS in clinical routine diagnostics—A three year recap from serum cortisol to dried blood 17α-hydroxyprogesterone. J Steroid Biochem Mol Biol. 2019; 192:105389.
26. Ma S, Guo Q, Zhang Z, He Z, Yue A, Song Z, et al. Expanded newborn screening for inborn errors of metabolism by tandem mass spectrometry in newborns from Xinxiang city in China. J Clin Lab Anal. 2020; 34:e23159.
27. Zhu Z, Gu J, Genchev G Z, Cai X, Wang Y, Guo J, et al. Improving the Diagnosis of Phenylketonuria by Using a Machine Learning-Based Screening Model of Neonatal MRM Data. Front Mol Biosci. 2020; 7:115.
28. Demichev V, Tober-Lau P, Nazarenko T, Aulakh S K, Whitwell H, Lemke O, et al. A proteomic survival predictor for COVID-19 patients in intensive care [Internet]. bioRxiv. medRxiv; 2021. Available from: http://medrxiv.org/lookup/doi/10.1101/2021.06.24.21259374
29. Shen B, Yi X, Sun Y, Bi X, Du J, Zhang C, et al. Proteomic and Metabolomic Characterization of COVID-19 Patient Sera. Cell. 2020; 182:59-72.e15.
30. MacLean B, Tomazela D M, Shulman N, Chambers M, Finney G L, Frewen B, et al. Skyline: an open source document editor for creating and analyzing targeted proteomics experiments. Bioinformatics. 2010; 26:966-8.
31. Jones B R, Schultz G A, Eckstein J A, Ackermann B L. Surrogate matrix and surrogate analyte approaches for definitive quantitation of endogenous biomolecules. Bioanalysis. 2012; 4:2343-56.
32. Pino L K, Searle B C, Yang H-Y, Hoofnagle A N, Noble W S, MacCoss M J. Matrix-Matched Calibration Curves for Assessing Analytical Figures of Merit in Quantitative Proteomics. J Proteome Res. 2020; 19:1147-53.
33. Hall T G, Smukste I, Bresciano K R, Wang Y, McKearn D, Savage R E. Identifying and overcoming matrix effects in drug discovery and development. Tandem mass spectrometry—applications and principles. Intech Open, London, U K; 2012; 18:390-419.
34. Xu R N, Fan L, Rieser M J, E I-Shourbagy T A. Recent advances in high-throughput quantitative bioanalysis by LC-MS/MS. J Pharm Biomed Anal. 2007; 44:342-55.
35. Gerber S A, Rush J, Stemman O, Kirschner M W, Gygi S P. Absolute quantification of proteins and phosphoproteins from cell lysates by tandem M S. Proc Natl Acad Sci USA. 2003; 100:6940-5.
36. Yang J J, Han Y, Mah C H, Wanjaya E, Peng B, Xu T F, et al. Streamlined MRM method transfer between instruments assisted with HRMS matching and retention-time prediction. Anal Chim Acta. 2020; 1100:88-96.
37. Anderson L, Hunter C L. Quantitative Mass Spectrometric Multiple Reaction Monitoring Assays for Major Plasma Proteins*. Mol Cell Proteomics. 2006; 5:573-88.
38. Fountoulakis M, Juranville J-F, Jiang L, Avila D, Roder D, Jakob P, et al. Depletion of the high-abundance plasma proteins. Amino Acids. 2004; 27:249-59.
39. Krieg L, Schaffert A, Kern M, Landgraf K, Wabitsch M, Beck-Sickinger A G, et al. An MRM-Based Multiplexed Quantification Assay for Human Adipokines and Apolipoproteins. Molecules [Internet]. 2020; 25. Available from: http://dx.doi.org/10.3390/molecules25040775
40. Brioschi M, Gianazza E, Agostoni P, Zoanni B, Mallia A, Banfi C. Multiplexed MRM-Based Proteomics Identified Multiple Biomarkers of Disease Severity in Human Heart Failure. Int J Mol Sci [Internet]. 2021; 22. Available from: http://dx.doi.org/10.3390/ijms22020838
41. World Health Organization [Internet]. R&D blueprint novel coronavirus COVID-19 therapeutic trial synopsis (WHO). 2020. Available from: https://www.who.int/teams/blueprint/covid-19
42. Bioanalytical-Method-Validation-Guidance-for-Industry.pdf. Available from: https://www.fda.gov/files/drugs/published/Bioanalytical-Method-Validation-Guidance-for-Industry.pdf
43. Benjamini Y, Hochberg Y. Controlling the False Discovery Rate: A Practical and Powerful Approach to Multiple Testing. J R Stat Soc Series B Stat Methodol. [Royal Statistical Society, Wiley]; 1995; 57:289-300.
44. Pedregosa F, Varoquaux G, Gramfort A, Michel V, Thirion B, Grisel O, et al. Scikit-learn: Machine Learning in Python. J Mach Learn Res. 2011; 12:2825-30.
45. Davidson-Pilon C. lifelines: survival analysis in Python. J Open Source Softw. The Open Journal; 2019; 4:1317.

Reference is made herein to the peptides and proteins of Table 1 which are as follows:

TABLE 1

| | | | | Corresponding heavy |
|---|---|---|---|---|
| | Protein Name | Protein Uniprot | Corresponding reference | isotope-labelled peptide |
| Protein Name | Abbreviation | Accesion Number | peptide sequence | internal standard sequence |
| Proteoglycan 4 | PRG4 | Q92954 | GLPNVVTSAISLPNIR | SILWRGLPNVVTSAISLPNIR(U-13C6, 15N4) |
| | | | (SEQ ID No.: 1) | (SEQ ID No.: 53) |
| Inter-alpha-trypsin | ITIH1 | P19827 | GHMLENHVER (SEQ ID | GHMLENHVER(U-13C6, 15N4)LWAYL |
| inhibitor heavy | | | No.: 2) | (SEQ ID No.: 54) |
| chain H1 | | | | |
| Plasminogen, | PLG | P00747 | EAQLPVIENK (SEQ ID | AGLLKEAQLPVIENK(U-13C6, 15N2) |
| EC 3.4.21.7 | | | No.: 3) | (SEQ ID No.: 55) |
| Plasminogen, | PLG | P00747 | CQSWSSMTPHR (SEQ ID | CQSWSSMTPHR(U-13C6, 15N4)HQK |
| EC 3.4.21.7 | | | No.: 4) | (SEQ ID No.: 56) |
| Actin, aortic smooth | ACTA2; | P62736; | EITALAPSTMK (SEQ ID | MQKEITALAPSTMK(U-13C6, 15N2) |
| muscle; Actin, | ACTB; | P60709; | No.: 5) | (SEQ ID No.: 57) |
| cytoplasmic 1; | ACTG1; | P63261; | | |
| Actin, cytoplasmic 2; | ACTG2 | P63267 | | |
| Actin, gamma-enteric | | | | |
| smooth muscle | | | | |
| Complement C1q | C1QC | P02747 | FNAVLTNPQGDYDTSTGK | NSLIRFNAVLTNPQGDYDTSTGK(U-13C6, |
| subcomponent | | | (SEQ ID No.: 6) | 15N2) (SEQ ID No.: 58) |
| subunit C | | | | |
| Complement C1q | C1QC | P02747 | TNQVNSGGVLLR (SEQ ID | TNQVNSGGVLLR(U-13C6, 15N4)LQVGE |
| subcomponent | | | No.: 7) | (SEQ ID No.: 59) |
| subunit C | | | | |
| Cystatin-C | CST3 | P01034 | ALDFAVGEYNK (SEQ ID | ALDFAVGEYNK(U-13C6, 15N2)ASNDM |
| | | | No.: 8) | (SEQ ID No.: 60) |
| Cystatin-C | CST3 | P01034 | LVGGPMDASVEEEGVRR | LVGGPMDASVEEEGVR(U-13C6, |
| | | | (SEQ ID No.: 9) | 15N4)RALDFA (SEQ ID No.: 61) |
| Protein ORM2 | ORM2 | Q06144 | SDVMYTDWK (SEQ ID No.: | SDVMYTDWK(U-13C6, 15N2)KDK |
| | | | 10) | (SEQ ID No.: 62) |
| Alpha-1- | SERPINA3 | P01011 | WEMPFDPQDTHQSR (SEQ ID | WEMPFDPQDTHQSR(U-13C6, 15N4)FYLSK |
| antichymotrypsin | | | No.: 11) | (SEQ ID No.: 63) |
| Alpha-1- | SERPINA3 | P01011 | EQLSLLDR (SEQ ID No.: 12) | EQLSLLDR(U-13C6, 15N4)FTEDA |
| antichymotrypsin | | | | (SEQ ID No.: 64) |
| Serotransferrin | TF | P02787 | GDVAFVK (SEQ ID No.: 13) | GDVAFVK(U-13C6, 15N2)HSTIF (SEQ ID |
| | | | | No.: 65) |
| Serotransferrin | TF | P02787 | WCALSHHER (SEQ ID No.: | PVKWCALSHHER(U-13C6, 15N4) (SEQ ID |
| | | | 14) | No.: 66) |
| Apolipoprotein B-100 | APOB | P04114 | IAELSATAQEIIK (SEQ ID | IAELSATAQEIIK(U-13C6, 15N2)SQAIA |
| | | | No.: 15) | (SEQ ID No.: 67) |
| Apolipoprotein B-100 | APOB | P04114 | EQHLFLPFSYK (SEQ ID No.: | EAICKEQHLFLPFSYK(U-13C6, 15N2) |
| | | | 16) | (SEQ ID No.: 68) |
| EGF-containing fibulin- | EFEMP1 | Q12805 | ADQVCINLR (SEQ ID | ADQVCINLR(U-13C6, 15N4)GSFAC |
| like extracellular | | | No.: 17) | (SEQ ID No.: 69) |
| matrix protein 1 | | | | |
| von Willebrand factor | VWF | P04275 | ILTSDVFQDCNK (SEQ ID | ILTSDVFQDCNK(U-13C6, 15N2)LVDPE |
| | | | No.: 18) | (SEQ ID No.: 70) |
| von Willebrand factor | VWF | P04275 | YAGSQVASTSEVLK (SEQ ID | YAGSQVASTSEVLK(U-13C6, 15N2)YTLFQ |
| | | | No.: 19) | (SEQ ID No.: 71) |
| C-reactive protein | CRP | P02741 | ESDTSYVSLK (SEQ ID | ESDTSYVSLK(U-13C6, 15N2)APLTK |
| | | | No.: 20) | (SEQ ID No.: 72) |
| C-reactive protein | CRP | P02741 | GYSIFSYATK (SEQ ID | GYSIFSYATK(U-13C6, 15N2)RQDNE |
| | | | No.: 21) | (SEQ ID No.: 73) |
| Lysozyme C, | LYZ | P61626 | STDYGIFQINSR (SEQ ID | STDYGIFQINSR(U-13C6, 15N4)YWCND |
| EC 3.2.1.17 | | | No.: 22) | (SEQ ID No.: 74) |
| Lysozyme C, | LYZ | P61626 | YWCNDGK (SEQ ID No.: 23) | YWCNDGK(U-13C6, 15N2)TPGAV |
| EC 3.2.1.17 | | | | (SEQ ID No.: 75) |
| Apolipoprotein A-I | APOA1 | P02647 | AHVDALR (SEQ ID No.: 24) | AHVDALR(U-13C6, 15N4)THLAP |
| | | | | (SEQ ID No.: 76) |
| Apolipoprotein A-I | APOA1 | P02647 | ATEHLSTLSEK (SEQ ID | EYHAKATEHLSTLSEK(U-13C6, 15N2) |
| | | | No.: 25) | (SEQ ID No.: 77) |
| Alpha-2-HS-glycoprotein | AHSG | P02765 | TVVQPSVGAAAGPVVPPCPGR | TVVQPSVGAAAGPVVPPCPGR(U-13C6, |
| | | | (SEQ ID No.: 26) | 15N4)IR (SEQ ID No.: 78) |
| Alpha-2-HS-glycoprotein | AHSG | P02765 | CNLLAEK (SEQ ID No.: 27) | CNLLAEK(U-13C6, 15N2)QYGFC |
| | | | | (SEQ ID No.: 79) |
| Histidine-rich | HRG | P04196 | GGEGTGYFVDFSVR (SEQ ID | GGEGTGYFVDFSVR(U-13C6, 15N4)NCPR |
| glycoprotein | | | No.: 28) | (SEQ ID No.: 80) |
| Histidine-rich | HRG | P04196 | IADAHLDR (SEQ ID No.: 29) | IADAHLDR(U-13C6, 15N4)VENTT |
| glycoprotein | | | | (SEQ ID No.: 81) |
| Beta-2-microglobulin | B2M | P61769 | VEHSDLSFSK (SEQ ID No.: | VEHSDLSFSK(U-13C6, 15N2)DWSFY |
| | | | 30) | (SEQ ID No.: 82) |
| Beta-2-microglobulin | B2M | P61769 | VNHVTLSQPK (SEQ ID No.: | EYACRVNHVTLSQPK(U-13C6, 15N2) |
| | | | 31) | (SEQ ID No.: 83) |
| N-acetylmuramoyl-L- | PGLYRP2 | Q96PD5 | GCPDVQASLPDAK (SEQ ID | PDATKGCPDVQASLPDAK(U-13C6, 15N2) |
| alanine amidase, EC | | | No.: 32) | (SEQ ID No.: 84) |
| 3.5.1.28 | | | | |
| N-acetylmuramoyl-L- | PGLYRP2 | Q96PD5 | TFTLLDPK (SEQ ID | TFTLLDPK(U-13C6, 15N2)ASLLT |
| alanine amidase, EC | | | No.: 33) | (SEQ ID No.: 85) |
| 3.5.1.28 | | | | |
| Transthyretin | TTR | P02766 | GSPAINVAVHVFR (SEQ ID | GSPAINVAVHVFR(U-13C6, 15N4)KAADD |
| | | | No.: 34) | (SEQ ID No.: 86) |
| Transthyretin | TTR | P02766 | AADDTWEPFASGK (SEQ ID | AADDTWEPFASGK(U-13C6, 15N2)TSESG |
| | | | No.: 35) | (SEQ ID No.: 87) |
| Plasma kallikrein, EC | KLKB1 | P03952 | DSVTGTLPK (SEQ ID | GCFLKDSVTGTLPK(U-13C6, 15N2) |
| 3.4.21.34 | | | No.: 36) | (SEQ ID No.: 88) |
| Plasma kallikrein, EC | KLKB1 | P03952 | IAYGTQGSSGYSLR (SEQ ID | IAYGTQGSSGYSLR(U-13C6, 15N4)LCNTG |
| 3.4.21.34 | | | No.: 37) | (SEQ ID No.: 89) |
| Antithrombin-III | SERPINC1 | P01008 | ANRPFLVFIR (SEQ ID | ANRPFLVFIR(U-13C6, 15N4)EVPLN |
| | | | No.: 38) | (SEQ ID No.: 90) |
| Heparin cofactor 2 | SERPIND1 | P05546 | TSCLLFMGR (SEQ ID | TSCLLFMGR(U-13C6, 15N4)VANPS |
| | | | No.: 39) | (SEQ ID No.: 91) |
| Afamin | AFM | P43652 | HFQNLGK (SEQ ID | QQECKHFQNLGK(U-13C6, 15N2) |
| | | | No.: 40) | (SEQ ID No.: 92) |
| Afamin | AFM | P43652 | TINPAVDHCCK (SEQ ID | TINPAVDHCCK(U-13C6, 15N2)TNFAF |
| | | | No.: 41) | (SEQ ID No.: 93) |
| Plasma protease C1 | SERPING1 | P05155 | LLDSLPSDTR (SEQ ID | LLDSLPSDTR(U-13C6, 15N4)LVLLN |
| inhibitor | | | No.: 42) | (SEQ ID No.: 94) |
| Plasma protease C1 | SERPING1 | P05155 | LVLLNAIYLSAK (SEQ ID | PSDTRLVLLNAIYLSAK(U-13C6, 15N2) |
| inhibitor | | | No.: 43) | (SEQ ID No.: 95) |
| Transferrin receptor | TFRC | P02786 | ILNIFGVIK (SEQ ID | ILNIFGVIK(U-13C6, 15N2)GFVEP |
| protein 1 | | | No.: 44) | (SEQ ID No.: 96) |
| Transferrin receptor | TFRC | P02786 | VSASPLLYTLIEK (SEQ ID | VSASPLLYTLIEK(U-13C6, 15N2)TMQNV |
| protein 1 | | | No.: 45) | (SEQ ID No.: 97) |
| Low affinity | FCGR3A | P08637 | DSGSYFCR (SEQ ID | DSGSYFCR(U-13C6, 15N4)GLFGS |
| immunoglobulin gamma | | | No.: 46) | (SEQ ID No.: 98) |
| Fc region receptor III-A | | | | |
| Monocyte differentiation | CD14 | P08571 | VLDLSCNR (SEQ ID | VLDLSCNR(U-13C6, 15N4)LNR |
| antigen CD14 | | | No.: 47) | (SEQ ID No.: 99) |
| Insulin-like growth | IGFALS | P35858 | DFALQNPSAVPR (SEQ ID | DFALQNPSAVPR(U-13C6, 15N4)FVQAI |
| factor-binding | | | No.: 48) | (SEQ ID No.: 100) |
| protein complex | | | | |
| acid labile subunit | | | | |
| Insulin-like growth | IGFALS | P35858 | LAELPADALGPLQR (SEQ ID | LAELPADALGPLQR(U-13C6, 15N4)AFWLD |
| factor-binding | | | No.: 49) | (SEQ ID No.: 101) |
| protein complex | | | | |
| acid labile subunit | | | | |
| Immunoglobulin heavy | IGHV5-51 | A0A0C4DH38 | ASDTAMYYCAR (SEQ ID | WSSLKASDTAMYYCAR(U-13C6, 15N4) |
| variable 5-51 | | | No.: 50) | (SEQ ID No.: 102) |
| Complement C3 | C3 | P01024 | VEGTAFVIFGIQDGEQR (SEQ | VEGTAFVIFGIQDGEQR(U-13C6, 15N4)ISLPE |
| | | | ID No.: 51) | (SEQ ID No.: 103) |
| Complement C3 | C3 | P01024 | VHQYFNVELIQPGAVK (SEQ | VHQYFNVELIQPGAVK(U-13C6, 15N2)VYAYY |
| | | | ID No.: 52) | (SEQ ID No.: 104) |

Table 2 shows experimentally optimised targeted LC-MS/MS conditions to monitor native peptides listed in Table 1. The data in Table 2 is based on AB Sciex triple quadruple LC-MS/MS platforms but is equally transferable to platforms manufactured by other companies. In Table 2, the following abbreviations are used: EP—Entrance Potential, CE—Collision Energy, CXP—Collision Cell Exit Potential, DP—Declustering Potential, and RT—retention time.

TABLE 2

| Native | Precursor | Product | Dwell | | | | | |
|---|---|---|---|---|---|---|---|---|
| Peptide | (Q1) | (Q3) | time | | | | | RT |
| Sequence | m/z | (ms) | (ms) | EP | CE | CXP | DP | (min) |
| VEHSDLSFSK | 383.52 | 581.33 | 3.5 | 10 | 19 | 25 | 120 | 3.44 |
| SEQ ID No.: 30) | 383.52 | 468.25 | 3.5 | 10 | 19 | 25 | 120 | 3.44 |
| VNHVTLSQPK | 374.88 | 673.39 | 3.5 | 10 | 18.6 | 25 | 120 | 3.08 |
| (SEQ ID No.: 31) | 374.88 | 572.34 | 3.5 | 10 | 18.6 | 25 | 120 | 3.08 |
| | 374.88 | 459.26 | 3.5 | 10 | 18.6 | 25 | 120 | 3.08 |
| GLPNVVTSAISLPNIR | 825.98 | 1170.68 | 3.5 | 10 | 43.3 | 25 | 176 | 6.58 |
| (SEQ ID No.: 1) | 825.98 | 1071.62 | 3.5 | 10 | 43.3 | 25 | 176 | 6.58 |
| | 825.98 | 499.30 | 3.5 | 10 | 31.3 | 25 | 176 | 6.58 |
| GHMLENHVER | 407.86 | 783.37 | 3.5 | 10 | 20.2 | 25 | 132 | 2.9 |
| (SEQ ID No.: 2) | 407.86 | 654.33 | 3.5 | 10 | 24.2 | 25 | 132 | 2.9 |
| | 407.86 | 403.23 | 3.5 | 10 | 24.2 | 25 | 132 | 2.9 |
| EAQLPVIENK | 570.82 | 699.40 | 3.5 | 10 | 26.5 | 25 | 142 | 4.38 |
| (SEQ ID No.: 3) | 570.82 | 503.28 | 3.5 | 10 | 34.5 | 25 | 142 | 4.38 |
| | 570.82 | 390.20 | 3.5 | 10 | 34.5 | 25 | 142 | 4.38 |
| CQSQSSMTPHR | 459.53 | 815.38 | 3.5 | 10 | 22.6 | 25 | 124 | 3.81 |
| (SEQ ID NO.: 4) | 459.53 | 510.28 | 3.5 | 10 | 22.6 | 25 | 124 | 3.81 |
| | 459.23 | 406.23 | 3.5 | 10 | 22.6 | 25 | 124 | 3.81 |
| EITALAPSTMK | 581.31 | 747.41 | 3.5 | 10 | 27.1 | 25 | 140 | 4.49 |
| (SEQ ID No.: 5) | 581.31 | 634.32 | 3.5 | 10 | 23.1 | 25 | 140 | 4.49 |
| | 581.31 | 563.29 | 3.5 | 10 | 23.1 | 25 | 140 | 4.49 |
| FNAVLTNPQGDYDTSTGK | 643.31 | 1168.51 | 3.5 | 10 | 46.2 | 25 | 142 | 4.67 |
| (SEQ ID No.: 6) | 643.31 | 584.76 | 3.5 | 10 | 54.2 | 25 | 142 | 4.67 |
| | 643.31 | 493.26 | 3.5 | 10 | 58.2 | 25 | 142 | 4.67 |
| TNQVNSGGVLLR | 629.35 | 914.54 | 3.5 | 10 | 33.5 | 25 | 152 | 4.16 |
| (SEQ ID No.: 7) | 629.35 | 815.47 | 3.5 | 10 | 33.5 | 25 | 152 | 4.16 |
| | 629.35 | 701.43 | 3.5 | 10 | 33.5 | 25 | 152 | 4.16 |
| ALDFAVGEYNK | 613.81 | 610.28 | 3.5 | 10 | 24..7 | 25 | 140 | 5.05 |
| (SEQ ID No.: 8) | 613.81 | 709.35 | 3.5 | 10 | 28.7 | 25 | 140 | 5.05 |
| | 613.81 | 610.28 | 3.5 | 10 | 24.7 | 25 | 140 | 5.05 |
| LVGGPMDASVEEEGVRR | 600.97 | 745.40 | 3.5 | 10 | 37.2 | 25 | 142 | 4.38 |
| (SEQ ID No.: 9) | 600.97 | 794.87 | 3.5 | 10 | 25.2 | 25 | 142 | 4.38 |
| | 600.97 | 566.29 | 3.5 | 10 | 37.2 | 25 | 142 | 4.38 |
| SDVMYTDWK | 572.75 | 843.37 | 3.5 | 10 | 26.6 | 25 | 140 | 4.65 |
| (SEQ ID No.: 10) | 572.75 | 712.33 | 3.5 | 10 | 26.6 | 25 | 140 | 4.65 |
| | 572.75 | 549.27 | 3.5 | 10 | 26.6 | 25 | 140 | 4.65 |
| WEMPFDPQDTHQSR | 591.93 | 968.45 | 3.5 | 10 | 32.8 | 25 | 138 | 5.34 |
| (SEQ ID No.: 11) | 591.93 | 628.32 | 3.5 | 10 | 40.8 | 25 | 138 | 5.34 |
| | 591.93 | 729.83 | 3.5 | 10 | 24.8 | 25 | 138 | 5.34 |
| EQLSLLDR | 487.27 | 603.35 | 3.5 | 10 | 22.4 | 25 | 134 | 4.69 |
| (SEQ ID No.: 12) | 487.27 | 603.35 | 3.5 | 10 | 22.4 | 25 | 134 | 4.69 |
| | 487.27 | 403.23 | 3.5 | 10 | 22.4 | 25 | 134 | 4.69 |
| GDVAFK | 368.21 | 563.36 | 3.5 | 10 | 16.4 | 25 | 112 | 3.69 |
| (SEQ ID No.: 13) | 368.21 | 464.29 | 3.5 | 10 | 16.4 | 25 | 112 | 3.69 |
| | 368.21 | 393.25 | 3.5 | 10 | 20.4 | 25 | 112 | 3.69 |
| WCALSHHER | 399.19 | 665.31 | 3.5 | 10 | 23.8 | 25 | 118 | 3.16 |
| (SEQ ID No.: 14) | 399.19 | 578.28 | 3.5 | 10 | 27.8 | 25 | 118 | 3.16 |
| | 399.19 | 441.22 | 3.5 | 10 | 27.8 | 25 | 118 | 3.16 |
| IAELSATAQEIIK | 693.90 | 960.54 | 3.5 | 10 | 32.7 | 25 | 164 | 5.32 |
| (SEQ ID No.: 15) | 693.90 | 873.50 | 3.5 | 10 | 32.7 | 25 | 164 | 5.32 |
| | 693.90 | 802.47 | 3.5 | 10 | 32.7 | 25 | 164 | 5.32 |
| EQHLFLPFSYK | 470.25 | 641.33 | 3.5 | 10 | 19.1 | 25 | 120 | 5.97 |
| (SEQ ID No. 16) | 470.25 | 544.28 | 3.5 | 10 | 23.1 | 25 | 10 | 5.97 |
| | 470.25 | 397.21 | 3.5 | 10 | 27.1 | 25 | 120 | 5.97 |
| ADQVCINLR | 544.78 | 774.43 | 3.5 | 10 | 29.2 | 25 | 134 | 4.32 |
| (SEQ ID No.: 17) | 544.78 | 675.36 | 3.5 | 10 | 29.2 | 25 | 134 | 4.32 |
| | 544.78 | 515.33 | 3.5 | 10 | 29.2 | 25 | 134 | 4.32 |
| ILTSDVFQDCNK | 720.35 | 811.34 | 3.5 | 10 | 34 | 25 | 162 | 4.74 |
| (SEQ ID No.: 18) | 720.35 | 664.27 | 3.5 | 10 | 34 | 25 | 162 | 4.74 |
| | 720.35 | 663.80 | 3.5 | 10 | 42 | 25 | 162 | 4.74 |
| ESDTSYVSLK | 564.77 | 696.39 | 3.5 | 10 | 26.2 | 25 | 144 | 3.89 |
| (SEQ ID No.: 20) | 564.77 | 609.36 | 3.5 | 10 | 26.2 | 25 | 144 | 3.89 |
| | 564.77 | 446.30 | 3.5 | 10 | 26.2 | 25 | 144 | 3.89 |
| GYSIFSYATK | 568.78 | 716.36 | 3.5 | 10 | 26.4 | 25 | 136 | 5.4 |
| (SEQ ID No.: 21) | 568.78 | 569.29 | 3.5 | 10 | 18.4 | 25 | 136 | 5.4 |
| | 568.78 | 482.26 | 3.5 | 10 | 38.4 | 25 | 136 | 5.4 |
| DTSYGIFQINSR | 700.84 | 934.51 | 3.5 | 10 | 37 | 25 | 86 | 5.37 |
| (SEQ ID No.: 22) | 700.84 | 764.40 | 3.5 | 10 | 33 | 25 | 86 | 5.37 |
| | 700.84 | 617.34 | 3.5 | 10 | 33 | 25 | 86 | 5.37 |
| YAGSQVASTSEVLK | 720.37 | 933.53 | 3.5 | 10 | 38 | 25 | 172 | 4.11 |
| (SEQ ID No.: 19) | 720.37 | 834.46 | 3.5 | 10 | 38 | 25 | 172 | 4.11 |
| | 720.37 | 763.42 | 3.5 | 10 | 38 | 25 | 172 | 4.11 |
| YWCNDGK | 471.69 | 779.31 | 3.5 | 10 | 21.6 | 25 | 132 | 2.88 |
| (SEQ ID No.: 23) | 471.69 | 593.23 | 3.5 | 10 | 21.6 | 25 | 132 | 2.88 |
| | 471.69 | 433.20 | 3.5 | 10 | 25.6 | 25 | 132 | 2.88 |
| AHVDALR | 391.22 | 573.34 | 3.5 | 10 | 21.6 | 25 | 132 | 2.39 |
| (SEQ ID No.: 24) | 391.22 | 474.27 | 3.5 | 10 | 25.6 | 25 | 132 | 2.39 |
| | 391.22 | 359.24 | 3.5 | 10 | 29.6 | 25 | 132 | 2.39 |
| ATEHLSTLSEK | 405.88 | 777.44 | 3.5 | 10 | 24.1 | 25 | 120 | 3.11 |
| (SEQ ID No.: 25) | 405.88 | 664.35 | 3.5 | 10 | 24.1 | 25 | 120 | 3.11 |
| | 405.88 | 363.13 | 3.5 | 10 | 24.1 | 25 | 120 | 3.11 |
| TVVQPSVGAAAGPVVPPCPGR | 672.69 | 978.52 | 3.5 | 10 | 24.6 | 25 | 146 | 4.96 |
| (SEQ ID No.: 26) | 672.69 | 683.33 | 3.5 | 10 | 28.6 | 25 | 146 | 4.96 |
| | 672.69 | 586.28 | 3.5 | 10 | 40.6 | 25 | 146 | 4.69 |
| GGEGTGYFVDFSVR | 745.85 | 1089.54 | 3.5 | 10 | 39.3 | 25 | 172 | 5.76 |
| (SEQ ID No.: 28) | 745.85 | 869.45 | 3.5 | 10 | 39.3 | 25 | 172 | 5.76 |
| | 745.85 | 508.29 | 3.5 | 10 | 39.3 | 25 | 172 | 5.76 |
| IADAHLDR | 455.74 | 797.39 | 3.5 | 10 | 24.8 | 25 | 144 | 2.69 |
| (SEQ ID No. 29) | 455.74 | 726.35 | 3.5 | 10 | 24.8 | 25 | 144 | 2.69 |
| | 455.74 | 611.33 | 3.5 | 10 | 28.58 | 25 | 144 | 2.69 |
| CNLLAEK | 424.22 | 687.40 | 3.5 | 10 | 23.2 | 25 | 128 | 3.55 |
| (SEQ IS No.: 27) | 424.22 | 573.36 | 3.5 | 10 | 23.2 | 25 | 128 | 3.55 |
| | 424.22 | 460.28 | 3.5 | 10 | 23.2 | 25 | 128 | 3.55 |
| GCPDVQASLPDAK | 453.22 | 630.35 | 3.5 | 10 | 28 | 25 | 120 | 4.06 |
| (SEQ ID No.: 32) | 453.22 | 430.23 | 3.5 | 10 | 32 | 25 | 120 | 4.06 |
| | 453.22 | 430.14 | 3.5 | 10 | 44 | 25 | 120 | 4.06 |
| TFTLLDPK | 467.77 | 686.41 | 3.5 | 10 | 21.4 | 25 | 132 | 5.26 |
| (SEQ ID No.: 33) | 467.77 | 585.36 | 3.5 | 10 | 21.4 | 25 | 132 | 5.26 |
| | 467.77 | 472.28 | 3.5 | 10 | 21.4 | 25 | 132 | 5.26 |
| GSPAINVAVHVFR | 456.26 | 728.42 | 3.5 | 10 | 26.4 | 25 | 122 | 5.4 |
| (SEQ ID No.: 34) | 456.26 | 558.31 | 3.5 | 10 | 18.4 | 25 | 122 | 5.4 |
| | 456.26 | 611.86 | 3.5 | 10 | 18.4 | 25 | 122 | 5.4 |
| AADDTWEPFASGK | 697.81 | 921.45 | 3.5 | 10 | 32.9 | 25 | 156 | 5.04 |
| (SEQ ID No.: 35) | 697.81 | 735.37 | 3.5 | 10 | 32.9 | 25 | 156 | 5.04 |
| | 697.81 | 606.32 | 3.5 | 10 | 40.9 | 25 | 156 | 5.04 |
| DSVTGTLPK | 459.25 | 616.37 | 3.5 | 10 | 21 | 25 | 128 | 3.56 |
| (SEQ ID No.: 36) | 459.25 | 515.32 | 3.5 | 10 | 21 | 25 | 128 | 3.56 |
| | 459.25 | 357.25 | 3.5 | 10 | 29 | 25 | 128 | 3.56 |
| IATGTQGSSGYSLR | 730.36 | 1112.53 | 3.5 | 10 | 38.5 | 25 | 166 | 4.19 |
| (SEQ ID No.: 37) | 730.36 | 954.46 | 3.5 | 10 | 38.5 | 25 | 166 | 4.19 |
| | 730.36 | 826.41 | 3.5 | 10 | 42.5 | 25 | 166 | 4.19 |
| ANRPFLVFIR | 411.58 | 647.42 | 3.5 | 10 | 20.3 | 25 | 190 | 6 |
| (SEQ ID No.: 38) | 411.58 | 534.34 | 3.5 | 10 | 20.3 | 25 | 190 | 6 |
| | 411.58 | 435.27 | 3.5 | 10 | 20.3 | 25 | 190 | 6 |
| TSCLLFMGR | 542.77 | 736.42 | 3.5 | 10 | 29.1 | 25 | 134 | 5.48 |
| (SEQ ID No.: 39) | 542.77 | 623.33 | 3.5 | 10 | 29.1 | 25 | 134 | 5.48 |
| | 542.77 | 510.25 | 3.5 | 10 | 25.1 | 25 | 134 | 5.48 |
| HFQNLGK | 422.23 | 706.39 | 3.5 | 10 | 23.1 | 25 | 126 | 2.54 |
| (SEQ ID No.: 40) | 422.23 | 559.32 | 3.5 | 10 | 23.1 | 25 | 126 | 2.54 |
| | 422.23 | 431.26 | 3.5 | 10 | 27.1 | 25 | 126 | 2.54 |
| TINPAVDHCCK | 438.87 | 719.26 | 3.5 | 10 | 25.6 | 25 | 120 | 2.92 |
| (SEQ ID No.: 41) | 438.87 | 604.23 | 3.5 | 10 | 25.6 | 25 | 120 | 2.92 |
| | 438.87 | 467.17 | 3.5 | 10 | 33.6 | 25 | 120 | 2.92 |
| LLDSLPSDTR | 558.80 | 890.42 | 3.5 | 10 | 25.9 | 25 | 140 | 4.38 |
| (SEQ ID No.: 42) | 558.80 | 575.28 | 3.5 | 10 | 25.9 | 25 | 140 | 4.38 |
| | 558.80 | 575.28 | 3.5 | 10 | 25.9 | 25 | 140 | 4.38 |
| LVLLNAIYLSAK | 659.41 | 879.49 | 3.5 | 10 | 31 | 25 | 148 | 6.82 |
| (SEQ ID No.: 43) | 659.41 | 765.45 | 3.5 | 10 | 31 | 25 | 148 | 6.82 |
| | 659.41 | 694.41 | 3.5 | 10 | 43 | 25 | 148 | 6.82 |
| ILNIFGVIK | 508.83 | 790.48 | 3.5 | 10 | 23.4 | 25 | 132 | 6.8 |
| (SEQ ID No.: 44) | 508.83 | 676.44 | 3.5 | 10 | 23.4 | 25 | 132 | 6.8 |
| | 508.83 | 563.36 | 3.5 | 10 | 23.4 | 25 | 132 | 6.8 |
| VSASPLLYTLIEK | 717.42 | 1089.66 | 3.5 | 10 | 29.9 | 25 | 156 | 6.66 |
| (SEQ ID No.: 45) | 717.42 | 879.52 | 3.5 | 10 | 37.9 | 25 | 156 | 6.66 |
| | 717.42 | 766.43 | 3.5 | 10 | 33.9 | 25 | 156 | 6.66 |
| DGSYFCR | 496.20 | 789.33 | 3.5 | 10 | 22.8 | 25 | 84 | 3.58 |
| (SEQ ID No.: 46) | 496.20 | 645.28 | 3.5 | 10 | 22.8 | 25 | 84 | 3.58 |
| | 496.20 | 482.22 | 3.5 | 10 | 22.8 | 25 | 84 | 3.58 |
| VLDLSCNR | 488.75 | 764.34 | 3.5 | 10 | 22.4 | 25 | 128 | 3.8 |
| (SEQ ID No.: 47) | 488.75 | 649.31 | 3.5 | 10 | 26.4 | 25 | 128 | 3.8 |
| | 488.75 | 536.22 | 3.5 | 10 | 22.4 | 25 | 128 | 3.8 |
| DFALQNPSAVPR | 657.84 | 868.46 | 3.5 | 10 | 34.9 | 25 | 110 | 4.84 |
| (SEQ ID No.: 48) | 657.84 | 740.40 | 3.5 | 10 | 30.9 | 25 | 110 | 4.84 |
| | 657.84 | 626.36 | 3.5 | 10 | 34.9 | 25 | 110 | 4.84 |
| LAELPADALGLQR | 732.41 | 10.37.57 | 3.5 | 10 | 34.6 | 25 | 88 | 5.77 |
| (SEQ ID No.: 49) | 732.41 | 754.46 | 3.5 | 10 | 50.6 | 25 | 88 | 5.77 |
| | 732.41 | 570.34 | 3.5 | 10 | 34.6 | 25 | 88 | 5.77 |
| ASDTAMYYCAR | 654.77 | 934.39 | 3.5 | 10 | 34.7 | 25 | 148 | 5.18 |
| (SEQ ID No.: 50) | 654.77 | 863.35 | 3.5 | 10 | 30.7 | 25 | 148 | 5.18 |
| | 654.77 | 732.31 | 3.5 | 10 | 30.7 | 25 | 148 | 5.18 |
| VEGTAFVIFGIQDGEQR | 622.65 | 732.33 | 3.5 | 10 | 26.3 | 25 | 142 | 6.46 |
| (SEQ ID No.: 51) | 622.65 | 604.27 | 3.55 | 10 | 30.3 | 25 | 142 | 6.46 |
| | 622.65 | 525.25 | 3.5 | 10 | 22.3 | 25 | 142 | 6.46 |
| VHQYFNVELIQPGAVK | 614.67 | 712.44 | 3.5 | 10 | 21.9 | 25 | 132 | 5.7 |
| (SEQ ID No.: 52) | 614.67 | 471.29 | 3.5 | 10 | 33.9 | 25 | 132 | 5.7 |
| | 614.67 | 509.24 | 3.5 | 10 | 21.9 | 25 | 132 | 5.7 |

Table 3 shows experimentally optimised targeted LC-MS/MS conditions to monitor heavy isotope-labelled peptide internal standards listed in Table 1. This is based on AB Sciex triple quadruple LC-MS/MS platforms.

TABLE 3

| Internal standard | Precusor | Product | Dwell | | | | | RT |
|---|---|---|---|---|---|---|---|---|
| peptide sequence | (Q1) m/z | (Q3) m/z | time (ms) | EP | CE | CXP | DP | (min) |
| VEHSDLSFSK(U-13C6, | 386.20 | 589.34 | 3.5 | 10 | 19 | 25 | 120 | 3.44 |
| 15N2)DWSFY | 386.20 | 476.25 | 3.5 | 10 | 19 | 25 | 120 | 3.44 |
| (SEQ ID No.: 82) | | | | | | | | |
| EYACRVNHVTLSQPK | 377.55 | 580.35 | 3.5 | 10 | 16.8 | 25 | 120 | 3.08 |
| (U-13C6, 15N2) | 377.55 | 467.27 | 3.5 | 10 | 18.6 | 25 | 120 | 3.08 |
| (SEQ ID No.: 83) | | | | | | | | |
| SILBRGLPNVVTSAISLPNIR | 830.98 | 1180.69 | 3.5 | 10 | 43.3 | 25 | 176 | 6.58 |
| (U-13C6, 15N4) | 830.98 | 1081.62 | 3.5 | 10 | 43.3 | 25 | 176 | 6.58 |
| (SEQ ID No.: 53) | | | | | | | | |
| GHMLENHVER | 411.20 | 493.38 | 3.5 | 10 | 20.2 | 25 | 132 | 2.9 |
| (U-13C6, 15N4)LWAYL | 411.20 | 413.23 | 3.5 | 10 | 24.2 | 25 | 132 | 2.9 |
| (SEQ ID No.: 54) | | | | | | | | |
| AGLLKEAQLPVIENK | 574.84 | 707.41 | 3.5 | 10 | 26.5 | 25 | 142 | 4.38 |
| (U-13C6, 15N4) | 574.84 | 511.30 | 3.5 | 10 | 34.5 | 25 | 142 | 4.38 |
| (SEQ ID No.: 55) | | | | | | | | |
| CQSWSSMTPHR | 462.86 | 520.28 | 3.5 | 10 | 22.6 | 25 | 124 | 3.81 |
| (U-13C6, 15N4)HQK | 462.86 | 419.23 | 3.5 | 10 | 22.6 | 25 | 124 | 3.81 |
| (SEQ ID No.: 56) | | | | | | | | |
| MQKEITALAPSTMK | 585.31 | 755.42 | 3.5 | 10 | 27.1 | 25 | 140 | 4.49 |
| (U-13C6, 15N4) | 585.31 | 642.33 | 3.5 | 10 | 23.1 | 25 | 140 | 4.49 |
| (SEQ ID No.: 57) | | | | | | | | |
| NSLIRFNAVLTNPQGDYDTSTGK | 645.97 | 1176.52 | 3.5 | 10 | 46.2 | 25 | 142 | 4.67 |
| (U-13C6, 15N4) | 645.97 | 588.76 | 3.5 | 10 | 54.2 | 25 | 142 | 4.67 |
| (SEQ ID No.: 58) | | | | | | | | |
| TNQVNSGGVLLR | 634.35 | 924.55 | 3.5 | 10 | 33.5 | 25 | 152 | 4.16 |
| (U-13C6, 15N4)LQVGE | 634.35 | 825.48 | 3.5 | 10 | 33.5 | 25 | 152 | 4.16 |
| (SEQ ID No.: 59) | | | | | | | | |
| ALDFAVGEYNK | 617.81 | 717.36 | 3.5 | 10 | 28.7 | 25 | 140 | 5.05 |
| (U-13C6, 15N4)ASNDM | 617.81 | 618.29 | 3.5 | 10 | 24.7 | 25 | 140 | 5.05 |
| (SEQ ID No.: 60) | | | | | | | | |
| LVGGPMDASVEEEGVR | 604.30 | 799.88 | 3.5 | 10 | 25.2 | 25 | 142 | 4.38 |
| (U-13C6, 15N4)RALDFA | 604.30 | 571.30 | 3.5 | 10 | 37.2 | 25 | 142 | 4.38 |
| (SEQ ID No.: | | | | | | | | |
| SDVMYTDWK | 576.75 | 851.38 | 3.5 | 10 | 26.6 | 25 | 140 | 4.65 |
| (U-13C6, 15N4)KDK | 576.75 | 720.34 | 3.5 | 10 | 26.6 | 25 | 140 | 4.65 |
| (SEQ ID No.: | | | | | | | | |
| WEMPFDPQDTHQSR | 595.26 | 978.46 | 3.5 | 10 | 32.8 | 25 | 138 | 5.34 |
| (U-13C6, 15N4)FYLSK | 595.26 | 734.82 | 3.5 | 10 | 24.8 | 25 | 138 | 5.34 |
| (SEQ ID No.: 63) | | | | | | | | |
| EQLSLLDR | 492.27 | 726.43 | 3.5 | 10 | 22.4 | 25 | 134 | 4.69 |
| FTEDA | 492.27 | 613.35 | 3.5 | 10 | 22.4 | 25 | 134 | 4.69 |
| (SEQ ID No.: 64) | | | | | | | | |
| GDVAFU | 372.21 | 571.36 | 3.5 | 10 | 16.4 | 25 | 112 | 3.69 |
| (U-13C6, 15N4)HSTIF | 372.21 | 472.30 | 3.5 | 10 | 16.4 | 25 | 112 | 3.69 |
| (SEQ ID No.: 65) | | | | | | | | |
| PVKWCALSHHER | 402.52 | 675.31 | 3.5 | 10 | 23.8 | 25 | 118 | 3.16 |
| (U-13C6, 15N4) (SEQ ID No.: 66) | 402.52 | 451.22 | 3.5 | 10 | 27.8 | 25 | 118 | 3.16 |
| IAELSATAQEIIK | 697.90 | 968.55 | 3.5 | 10 | 32.7 | 25 | 164 | 5.32 |
| (U-13C6, 15N4)SQAIA | 697.90 | 810.48 | 3.5 | 10 | 32.7 | 25 | 164 | 5.32 |
| (SEQ ID No.: 67) | | | | | | | | |
| EAICKEQHLFLPFSYK | 472.91 | 649.34 | 3.5 | 10 | 19.1 | 25 | 120 | 5.97 |
| (U-13C6, 15N4) (SEQ ID No.: 68) | 472.91 | 552.29 | 3.5 | 10 | 23.1 | 25 | 120 | 5.97 |
| ADQVCINLR | 549.78 | 784.43 | 3.5 | 10 | 29.2 | 25 | 134 | 4.32 |
| (U-13C6, 15N4)GSFAC | 549.78 | 685.36 | 3.5 | 10 | 29.2 | 25 | 134 | 4.32 |
| (SEQ ID No.: 69) | | | | | | | | |
| ILTSDVFQDCNK | 724.35 | 819.35 | 3.5 | 10 | 34 | 25 | 162 | 4.74 |
| (U-13C6, 15N4)LVDPE | 724.35 | 677.81 | 3.5 | 10 | 42 | 25 | 162 | 4.74 |
| (SEQ ID No.: 70) | | | | | | | | |
| ESDTSYVSLK | 568.78 | 704.40 | 3.5 | 10 | 26.2 | 25 | 144 | 3.89 |
| (U-13C6, 15N4)APLTK | 568.78 | 617.37 | 3.5 | 10 | 26.2 | 25 | 144 | 3.89 |
| (SEQ ID No.: 72) | | | | | | | | |
| GYSIFSYATK | 572.79 | 724.37 | 3.5 | 10 | 26.4 | 25 | 136 | 5.4 |
| (U-13C6, 15N4)RQDNE | 572.79 | 577.30 | 3.5 | 10 | 18.4 | 25 | 136 | 5.4 |
| (SEQ ID No.: 73) | | | | | | | | |
| STDYGIFQINSR | 705.84 | 944.51 | 3.5 | 10 | 37 | 25 | 86 | 5.37 |
| (U-13C6, 15N4)YWCND | 705.84 | 774.41 | 3.5 | 10 | 33 | 25 | 86 | 5.37 |
| (SEQ ID No.: 74) | | | | | | | | |
| YAGSQVASTSEVLK | 724.37 | 941.53 | 3.5 | 10 | 38 | 25 | 172 | 4.11 |
| (U-13C6, 15N4)YTLFQ | 724.37 | 842.47 | 3.5 | 10 | 38 | 25 | 172 | 4.11 |
| (SEQ ID No.: 71) | | | | | | | | |
| YWCNDGK | 475.70 | 787.32 | 3.5 | 10 | 21.6 | 25 | 132 | 2.88 |
| (U-13C6, 15N4)TPGAV | 475.70 | 601.24 | 3.5 | 10 | 21.6 | 25 | 132 | 2.88 |
| (SEQ ID No.: 75) | | | | | | | | |
| AHVDAR | 396.22 | 583.34 | 3.5 | 10 | 21.6 | 25 | 132 | 2.39 |
| (U-13C6, 15N4)THLAP | 396.22 | 484.27 | 3.5 | 10 | 25.6 | 25 | 132 | 2.39 |
| (SEQ ID No.: 76) | | | | | | | | |
| EYHAKATEHLSTLSEK | 408.55 | 672.36 | 3.5 | 10 | 24.1 | 25 | 120 | 3.11 |
| (U-13C6, 15N4) | | | | | | | | |
| (SEQ ID No.: 77) | | | | | | | | |
| TVVQPSVGAAAGPVVPPCPGR | 676.03 | 988.52 | 3.5 | 10 | 24.6 | 25 | 146 | 4.96 |
| (U-13C6, 15N4)IR | 676.03 | 693.33 | 3.5 | 10 | 28.6 | 25 | 146 | 4.96 |
| (SEQ ID No.: 78) | | | | | | | | |
| GGEGTGYFVDFSVR | 750.86 | 1099.54 | 3.5 | 10 | 39.3 | 25 | 172 | 5.76 |
| (U-13C6, 15N4)NCPR | 750.86 | 879.45 | 3.5 | 10 | 39.3 | 25 | 172 | 5.76 |
| (SEQ ID No.: 80) | | | | | | | | |
| IADAHLDR | 460.74 | 807.40 | 3.5 | 10 | 24.8 | 25 | 144 | 2.69 |
| (U-13C6, 15N4)VENTT | 460.74 | 736.36 | 3.5 | 10 | 24.8 | 25 | 144 | 2.69 |
| (SEQ ID No.: 81) | | | | | | | | |
| CNLLAEK | 428.22 | 581.37 | 3.5 | 10 | 23.2 | 25 | 128 | 3.55 |
| (U-13C6, 15N4)QYGFC | 428.22 | 468.30 | 3.5 | 10 | 23.2 | 25 | 128 | 3.55 |
| (SEQ ID No.: 79) | | | | | | | | |
| PDATKGCPDVQASLPDAK | 455.89 | 638.35 | 3.5 | 10 | 28 | 25 | 120 | 4.06 |
| (U-13C6, 15N4) | 455.89 | 438.24 | 3.5 | 10 | 32 | 25 | 120 | 4.06 |
| (SEQ ID No.: 84) | | | | | | | | |
| TFTLLDPK | 471.77 | 694.42 | 3.5 | 10 | 21.4 | 25 | 132 | 5.26 |
| (U-13C6, 15N4)ASLLT | 471.77 | 593.37 | 3.5 | 10 | 21.4 | 25 | 132 | 5.26 |
| (SEQ ID No.: 85) | | | | | | | | |
| GSPAINVAVHVFR | 459.59 | 738.42 | 3.5 | 10 | 26.4 | 25 | 122 | 5.4 |
| (U-13C6, 15N4)KAADD | 459.59 | 616.86 | 3.5 | 10 | 18.4 | 25 | 122 | 5.4 |
| (SEQ ID No.: 86) | | | | | | | | |
| AADDTWEPFASGK | 701.82 | 929.46 | 3.5 | 10 | 32.9 | 25 | 156 | 5.04 |
| (U-13C6, 15N4)TSESG | 701.82 | 614.33 | 3.5 | 10 | 40.9 | 25 | 156 | 5.04 |
| (SEQ ID No.: 87) | | | | | | | | |
| GCFLKDSVTGTLPK | 463.25 | 624.38 | 3.5 | 10 | 21 | 25 | 128 | 3.56 |
| (U-13C6, 15N4) | 463.25 | 523.33 | 3.5 | 10 | 21 | 25 | 128 | 3.56 |
| (SEQ ID No.: 88) | | | | | | | | |
| IAYGTQGSSGYSLR | 735.36 | 1122.54 | 3.5 | 10 | 38.5 | 25 | 166 | 4.19 |
| (U-13C6, 15N4)LCNTG | 735.36 | 836.41 | 3.5 | 10 | 42.5 | 25 | 166 | 4.19 |
| (SEQ ID No.: 89) | | | | | | | | |
| ANRPFLVFIR | 414.91 | 544.34 | 3.5 | 10 | 20.3 | 25 | 190 | 6 |
| (U-13C6, 15N4)EVPLN | 414.91 | 445.27 | 3.5 | 10 | 20.3 | 25 | 190 | 6 |
| (SEQ ID No.: 90) | | | | | | | | |
| TSCLLFMGR | 547.77 | 633.34 | 3.5 | 10 | 29.1 | 25 | 134 | 5.48 |
| (U-13C6, 15N4)VANPS | 547.77 | 520.25 | 3.5 | 10 | 25.1 | 25 | 134 | 5.48 |
| (SEQ ID No.: 91) | | | | | | | | |
| QQECKHFQNLGK | 426.23 | 714.40 | 3.5 | 10 | 23.1 | 25 | 126 | 2.54 |
| (U-13C6, 15N4) | 426.23 | 567.33 | 3.5 | 10 | 23.1 | 25 | 126 | 2.54 |
| (SEQ ID No.: 92) | | | | | | | | |
| TINPAVDHCCK | 441.54 | 727.27 | 3.5 | 10 | 25.6 | 25 | 120 | 2.92 |
| (U-13C6, 15N4)TNFAF | 441.54 | 612.24 | 3.5 | 10 | 25.6 | 25 | 120 | 2.92 |
| (SEQ ID No.: 93) | | | | | | | | |
| LLDSLPSDTR | 563.80 | 900.42 | 3.5 | 10 | 25.9 | 25 | 140 | 4.38 |
| (U-13C6, 15N4)LVLLN | 563.80 | 585.28 | 3.5 | 10 | 25.9 | 25 | 140 | 4.38 |
| (SEQ ID No.: 94) | | | | | | | | |
| PSDTRLVLLNAIYLSAK | 663.41 | 887.50 | 3.5 | 10 | 31 | 25 | 148 | 6.82 |
| (U-13C6, 15N4) | 663.42 | 773.46 | 3.55 | 10 | 31 | 25 | 148 | 6.82 |
| (SEQ ID No.: 95) | | | | | | | | |
| ILNIFGVIK | 512.83 | 798.49 | 3.5 | 10 | 23.4 | 25 | 132 | 6.8 |
| (U-13C6, 15N4)GFVEP | 512.83 | 571.36 | 3.5 | 10 | 23.4 | 25 | 132 | 6.8 |
| (SEQ ID No.: 96) | | | | | | | | |
| VSASPLLTYLIEK | 721.42 | 1097.66 | 3.5 | 10 | 29.9 | 25 | 156 | 6.66 |
| (U-13C6, 15N4)TMQNV | 721.42 | 774.44 | 3.5 | 10 | 33.9 | 25 | 1556 | 6.66 |
| (SEQ ID No.: 97) | | | | | | | | |
| DSGSYFCR | 501.20 | 799.34 | 3.5 | 10 | 22.8 | 25 | 84 | 3.58 |
| (U-13C6, 15N4)GLFGS | 501.20 | 655.28 | 3.5 | 10 | 22.8 | 25 | 84 | 3.58 |
| (SEQ ID No.: 98) | | | | | | | | |
| VLDLSCNR | 493.75 | 774.34 | 3.5 | 10 | 22.4 | 25 | 128 | 3.8 |
| (U-13C6, 15N4)LNR | 493.75 | 546.23 | 3.5 | 10 | 22.4 | 25 | 128 | 3.8 |
| (SEQ ID No.: 99) | | | | | | | | |
| DFALQNPSAVPR | 662.84 | 750.41 | 3.5 | 10 | 30.9 | 25 | 110 | 4.84 |
| (U-13C6, 15N4)FVQAI | 662.84 | 636.37 | 3.5 | 10 | 34.9 | 25 | 110 | 4.84 |
| (SEQ ID No.: 100) | | | | | | | | |
| LAELPADALGPLQR | 737.41 | 1047.52 | 3.5 | 10 | 34.6 | 25 | 88 | 5.77 |
| (U-13C6, 15N4)AFWLD | 737.41 | 764.46 | 3.5 | 10 | 50.6 | 25 | 88 | 5.77 |
| (SEQ ID No.: 101) | | | | | | | | |
| WSSLKASDTAMYYCAR | 659.78 | 873.36 | 3.5 | 10 | 30.7 | 25 | 148 | 5.18 |
| (U-13C6, 15N4) | 659.78 | 742.32 | 3.5 | 10 | 30.7 | 25 | 148 | 5.18 |
| (SEQ ID No.: 102) | | | | | | | | |
| VEGTAFVIFGIQDGEQR | 625.98 | 742.33 | 3.5 | 10 | 26.3 | 25 | 142 | 6.46 |
| (U-13C6, 15N4)ISLPE | 625.98 | 530.25 | 3.5 | 10 | 22.3 | 25 | 142 | 6.46 |
| (SEQ ID No.: 103) | | | | | | | | |
| VHQYFNVELIQPGAVK | 617.33 | 720.44 | 3.5 | 10 | 21.9 | 25 | 132 | 5.7 |
| (U-13C6, 15N4)VYAYY | 617.33 | 479.30 | 3.5 | 10 | 33.9 | 25 | 132 | 5.7 |
| (SEQ ID No.: 104) | | | | | | | | |

Table 4 shows peptides in the targeted LC-MS/MS assay ranked by ANOVA p-value. Top 17 peptides are essential for the COVID-19 assay as they show statistically significant concentration changes at different severity grades of COVID-19 disease. The top 17 peptides can be further ranked according to their p-value where the smallest p-value indicates the most important contribution to COVID-19 disease severity classification and prediction. Other peptides in the table also contribute to the overall assay performance and can also be ranked based on their P-value.

TABLE 4

| | ANOVA |
|---|---|
| Peptide | p-value |
| EQHLFLPFSYK (SEQ ID No.: 16) | 0.0000 |
| VLDLSCNR (SEQ ID No.: 47) | 0.0000 |
| GYSIFSYATK (SEQ ID No.: 21) | 0.0000 |
| LVLLNAIYLSAK (SEQ ID No.: 43) | 0.0001 |
| IADAHLDR (SEQ ID No.: 29) | 0.0001 |
| TFTLLDPK (SEQ ID No.: 33) | 0.0005 |
| GCPDVQASLPDAK (SEQ ID No.: 32) | 0.0008 |
| EITALAPSTMK (SEQ ID No.: 5) | 0.0020 |
| LLDSLPSDTR (SEQ ID No.: 42) | 0.0020 |
| EQLSLLDR (SEQ ID No.: 12) | 0.0050 |
| CNLLAEK (SEQ ID No.: 27) | 0.0080 |
| GLPNVVTSAISLPNIR (SEQ ID No.: 1) | 0.0100 |
| WCALSHHER (SEQ ID No.: 14) | 0.0140 |
| ADQVCINLR (SEQ ID No.: 17) | 0.0180 |
| GSPAINVAVHVFR (SEQ ID No.: 34) | 0.0380 |
| AHVDALR (SEQ ID No.: 24) | 0.0450 |
| TINPAVDHCCK (SEQ ID No.: 41) | 0.0470 |
| TVVQPSVGAAAGPVVPPCPGR (SEQ ID No.: 26) | 0.0630 |
| ATEHLSTLSEK (SEQ ID No.: 25) | 0.0640 |
| TSCLLFMGR (SEQ ID No.: 39) | 0.0920 |
| GGEGTGYFVDFSVR (SEQ ID No.: 28) | 0.1190 |
| YWCNDGK (SEQ ID No.: 23) | 0.1400 |
| ALDFAVGEYNK (SEQ ID No.: 8) | 0.1890 |
| HFQNLGK (SEQ ID No.: 40) | 0.2470 |
| DSVTGTLPK (SEQ ID No.: 36) | 0.2840 |
| ILNIFGVIK (SEQ ID No.: 44) | 0.3130 |
| ILTSDVFQDCNK (SEQ ID No.: 18) | 0.3380 |
| AADDTWEPFASGK (SEQ ID No.: 35) | 0.3720 |
| TNQVNSGGVLLR (SEQ ID No.: 7) | 0.3740 |
| DSGSYFCR (SEQ ID No.: 46) | 0.4240 |
| GHMLENHVER (SEQ ID No.: 2) | 0.4730 |
| SDVMYTDWK (SEQ ID No.: | 0.4920 |
| CQSWSSMTPHR (SEQ ID No.: 4) | 0.6180 |
| ANRPFLVFIR (SEQ ID No.: 38) | 0.6510 |
| EAQLPVIENK (SEQ ID No.: 3) | 0.7330 |
| FNAVLTNPQGDYDTSTGK (SEQ ID No.: 6) | 0.8770 |

TABLE 5

| | | | | Corresponding heavy |
|---|---|---|---|---|
| | | Protein Uniprot | Corresponding native | isotope-labelled internal |
| Protein Name | Gene name | Accesion Number | peptide sequence | standard peptide sequence |
| Proteoglycan 4 | PRG4 | Q92954 | GLPNVVTSAISLPNIR | SILWRGLPNVVTSAISLPNIR(U-13C6, |
| | | | (SEQ ID No.: 1) | 15N4) (SEQ ID No.: 53) |
| Inter-alpha-trypsin | ITIH1 | P19827 | GHMLENHVER (SEQ ID No.: | GHMLENHVER(U-13C6, |
| inhibitor heavy chain H1 | | | 2) | 15N4)LWAYL (SEQ ID No.: 54) |
| Plasminogen, EC 3.4.21.7 | PLG | P00747 | EAQLPVIENK (SEQ ID No.: | AGLLKEAQLPVIENK(U-13C6, |
| | | | 3) | 15N2) (SEQ ID No.: 55) |
| | | | CQSWSSMTPHR (SEQ ID | CQSWSSMTPHR(U-13C6, |
| | | | No.: 4) | 15N4)HQK (SEQ ID No.: 56) |
| Actin, aortic smooth | ACTA2; | P62736; | EITALAPSTMK (SEQ ID | MQKEITALAPSTMK(U-13C6, |
| muscle; Actin, cytoplasmic | ACTB; | P60709; | No.: 5) | 15N2) (SEQ ID No.: 57) |
| 1; Actin, cytoplasmic 2; | ACTG1; | P63261; | | |
| Actin, gamma-enteric | ACTG2 | P63267 | | |
| smooth muscle | | | | |
| Complement C1q | C1QC | P02747 | FNAVLTNPQGDYDTSTGK | NSLIRFNAVLTNPQGDYDTSTGK(U- |
| subcomponent subunit C | | | (SEQ ID No.: 6) | 13C6, 15N2) (SEQ ID No.: 58) |
| | | | TNQVNSGGVLLR (SEQ ID | TNQVNSGGVLLR(U-13C6, |
| | | | No.: 7) | 15N4)LQVGE (SEQ ID No.: 59) |
| Cystatin-C | CST3 | P01034 | ALDFAVGEYNK (SEQ ID | ALDFAVGEYNK(U-13C6, |
| | | | No.: 8) | 15N2)ASNDM (SEQ ID No.: 60) |
| | | | LVGGPMDASVEEEGVRR | LVGGPMDASVEEEGVR(U-13C6, |
| | | | (SEQ ID No.: 9) | 15N4)RALDFA (SEQ ID No.: 61) |
| Protein ORM2 | ORM2 | Q06144 | SDVMYTDWK (SEQ ID No.: | SDVMYTDWK(U-13C6, |
| | | | 10) | 15N2)KDK (SEQ ID No.: 62) |
| Alpha-1-antichymotrypsin | SERPINA3 | P01011 | WEMPFDPQDTHQSR (SEQ | WEMPFDPQDTHQSR(U-13C6, |
| | | | ID No.: 11) | 15N4)FYLSK (SEQ ID No.: 63) |
| | | | EQLSLLDR (SEQ ID No.: 12) | EQLSLLDR(U-13C6, |
| | | | | 15N4)FTEDA (SEQ ID No.: 64) |
| Serotransferrin | TF | P02787 | GDVAFVK (SEQ ID No.: 13) | GDVAFVK(U-13C6, |
| | | | | 15N2)HSTIF (SEQ ID No.: 65) |
| | | | WCALSHHER (SEQ ID No.: | PVKWCALSHHER(U-13C6, |
| | | | 14) | 15N4) (SEQ ID No.: 66) |
| Apolipoprotein B-100 | APOB | P04114 | IAELSATAQEIIK (SEQ ID No.: | IAELSATAQEIIK(U-13C6, |
| | | | 15) | 15N2)SQAIA (SEQ ID No.: 67) |
| | | | EQHLFLPFSYK (SEQ ID No.: | EAICKEQHLFLPFSYK(U-13C6, |
| | | | 16) | 15N2) (SEQ ID No.: 68) |
| EGF-containing fibulin-like | EFEMP1 | Q12805 | ADQVCINLR (SEQ ID No.: | ADQVCINLR(U-13C6, |
| extracellular matrix | | | 17) | 15N4)GSFAC (SEQ ID No.: 69) |
| protein 1 | | | | |
| von Willebrand factor | VWF | P04275 | ILTSDVFQDCNK (SEQ ID | ILTSDVFQDCNK(U-13C6, |
| | | | No.: 18) | 15N2)LVDPE (SEQ ID No.: 70) |
| | | | YAGSQVASTSEVLK (SEQ ID | YAGSQVASTSEVLK(U-13C6, |
| | | | No.: 19) | 15N2)YTLFQ (SEQ ID No.: 71) |
| C-reactive protein | CRP | P02741 | ESDTSYVSLK (SEQ ID No.: | ESDTSYVSLKAPLTK(U-13C6, |
| | | | 20) | 15N2) (SEQ ID No.: 72) |
| | | | GYSIFSYATK (SEQ ID No.: | GYSIFSYATK(U-13C6, |
| | | | 21) | 15N2)RQDNE (SEQ ID No.: 73) |
| Lysozyme C, EC 3.2.1.17 | LYZ | P61626 | STDYGIFQINSR (SEQ ID No.: | STDYGIFQINSR(U-13C6, |
| | | | 22) | 15N4)YWCND (SEQ ID No.: 74) |
| | | | YWCNDGK (SEQ ID No.: 23) | YWCNDGK(U-13C6, |
| | | | | 15N2)TPGAV (SEQ ID No.: 75) |
| Apolipoprotein A-I | APOA1 | P02647 | AHVDALR (SEQ ID No.: 24) | AHVDALR(U-13C6, |
| | | | | 15N4)THLAP (SEQ ID No.: 76) |
| | | | ATEHLSTLSEK (SEQ ID No.: | EYHAKATEHLSTLSEK(U-13C6, |
| | | | 25) | 15N2) (SEQ ID No.: 77) |
| Alpha-2-HS-glycoprotein | AHSG | P02765 | TVVQPSVGAAAGPVVPPCPGR | TVVQPSVGAAAGPVVPPCPGR(U- |
| | | | (SEQ ID No.: 26) | 13C6, 15N4)IR (SEQ ID No.: 78) |
| | | | CNLLAEK (SEQ ID No.: 27) | CNLLAEK(U-13C6, |
| | | | | 15N2)QYGFC (SEQ ID No.: 79) |
| Histidine-rich glycoprotein | HRG | P04196 | GGEGTGYFVDFSVR (SEQ ID | GGEGTGYFVDFSVR(U-13C6, |
| | | | No.: 28) | 15N4)NCPR (SEQ ID No.: 80) |
| | | | IADAHLDR (SEQ ID No.: 29) | IADAHLDR(U-13C6, |
| | | | | 15N4)VENTT (SEQ ID No.: 81) |
| N-acetylmuramoyl-L- | PGLYRP2 | Q96PD5 | GCPDVQASLPDAK (SEQ ID | PDATKGCPDVQASLPDAK(U-13C6, |
| alanine amidase, EC | | | No.: 32) | 15N2) (SEQ ID No.: 84) |
| 3.5.1.28 | | | TFTLLDPK (SEQ ID No.: 33) | TFTLLDPK(U-13C6, |
| | | | | 15N2)ASLLT (SEQ ID No.: 85) |
| Transthyretin | TTR | P02766 | GSPAINVAVHVFR (SEQ ID | GSPAINVAVHVFR(U-13C6, |
| | | | No.: 34) | 15N4)KAADD (SEQ ID No.: 86) |
| | | | AADDTWEPFASGK (SEQ ID | AADDTWEPFASGK(U-13C6, |
| | | | No.: 35) | 15N2)TSESG (SEQ ID No.: 87) |
| Plasma kallikrein, EC | KLKB1 | P03952 | DSVTGTLPK (SEQ ID No.: | GCFLKDSVTGTLPK(U-13C6, |
| 3.4.21.34 | | | 36) | 15N2) (SEQ ID No.: 88) |
| | | | IAYGTQGSSGYSLR (SEQ ID | IAYGTQGSSGYSLR(U-13C6, |
| | | | No.: 37) | 15N4)LCNTG (SEQ ID No.: 89) |
| Antithrombin-III | SERPINC1 | P01008 | ANRPFLVFIR (SEQ ID No.: | ANRPFLVFIR(U-13C6, |
| | | | 38) | 15N4)EVPLN (SEQ ID No.: 90) |
| Heparin cofactor 2 | SERPIND1 | P05546 | TSCLLFMGR (SEQ ID No.: | TSCLLFMGR(U-13C6, |
| | | | 39) | 15N4)VANPS (SEQ ID No.: 91) |
| Afamin | AFM | P43652 | HFQNLGK (SEQ ID No.: 40) | QQECKHFQNLGK(U-13C6, |
| | | | | 15N2) (SEQ ID No.: 92) |
| | | | TINPAVDHCCK (SEQ ID No.: | TINPAVDHCCK(U-13C6, |
| | | | 41) | 15N2)TNFAF (SEQ ID No.: 93) |
| Plasma protease C1 | SERPING1 | P05155 | LLDSLPSDTR (SEQ ID No.: | LLDSLPSDTR(U-13C6, |
| inhibitor | | | 42) | 15N4)LVLLN (SEQ ID No.: 94) |
| | | | LVLLNAIYLSAK (SEQ ID No.: | PSDTRLVLLNAIYLSAK(U-13C6, |
| | | | 43) | 15N2) (SEQ ID No.: 95) |
| Transferrin receptor | TFRC | P02786 | ILNIFGVIK (SEQ ID No.: 44) | ILNIFGVIK(U-13C6, |
| protein 1 | | | | 15N2)GFVEP (SEQ ID No.: 96) |
| | | | VSASPLLYTLIEK (SEQ ID No.: | VSASPLLYTLIEK(U-13C6, |
| | | | 45) | 15N2)TMQNV (SEQ ID No.: 97) |
| Low affinity | FCGR3A | P08637 | DSGSYFCR (SEQ ID No.: 46) | DSGSYFCR(U-13C6, |
| immunoglobulin gamma Fc | | | | 15N4)GLFGS (SEQ ID No.: 98) |
| region receptor III-A | | | | |
| Monocyte differentiation | CD14 | P08571 | VLDLSCNR (SEQ ID No.: 47) | VLDLSCNR(U-13C6, |
| antigen CD14 | | | | 15N4)LNR (SEQ ID No.: 99) |
| Insulin-like growth factor- | IGFALS | P35858 | DFALQNPSAVPR (SEQ ID | DFALQNPSAVPR(U-13C6, |
| binding protein complex | | | No.: 48) | 15N4)FVQAI (SEQ ID No.: 100) |
| acid labile subunit | | | LAELPADALGPLQR (SEQ ID | LAELPADALGPLQR(U-13C6, |
| | | | No.: 49) | 15N4)AFWLD (SEQ ID No.: 101) |
| Immunoglobulin heavy | IGHV5-51 | A0A0C4DH38 | ASDTAMYYCAR (SEQ ID | WSSLKASDTAMYYCAR(U-13C6, |
| variable 5-51 | | | No.: 50) | 15N4) (SEQ ID No.: 102) |
| Complement C3 | C3 | P01024 | VEGTAFVIFGIQDGEQR (SEQ | VEGTAFVIFGIQDGEQR(U-13C6, |
| | | | ID No.: 51) | 15N4)ISLPE (SEQ ID No.: 103) |
| | | | VHQYFNVELIQPGAVK (SEQ | VHQYFNVELIQPGAVK(U-13C6, |
| | | | ID No.: 52) | 15N2)VYAYY (SEQ ID No.: 104) |

Table 6 shows native and labelled peptide MRM transitions on 6495c (Agilent) LC-MS/MS platform. Internal standard (labelled) peptide sequences are shown in their post-digestion form (without tryptic tags). In table 6 the following abbreviation is used: CE—collision energy.

TABLE 6

| Peptide | | | | Dwell | | | Cell | |
|---|---|---|---|---|---|---|---|---|
| Standard | Standard | Precursor | Product | time | CE | | Accelerator | RT |
| Sequence | type | (m/z) | (m/z) | (ms) | (eV) | Fragmentor | Voltage | (min) |
| GLPNVVTSAISLPNIR | Native | 825.98 | 740.93 | 12.92 | 26.60 | 166.00 | 5.00 | 1.60 |
| (SEQ ID No.: 1) | | 825.98 | 499.30 | 12.92 | 26.60 | 166.00 | 5.00 | 1.60 |
| | | 825.98 | 1170.68 | 12.92 | 26.60 | 166.00 | 5.00 | 1.60 |
| GLPNVVTSAISLPNIR | Labelled | 830.98 | 745.93 | 12.92 | 26.60 | 166.00 | 5.00 | 1.60 |
| (U-13C6, 15N4) | | 830.98 | 509.31 | 12.92 | 26.60 | 166.00 | 5.00 | 1.60 |
| (SEQ ID No.: 53) | | | | | | | | |
| GHMLENHVER | Native | 407.86 | 514.25 | 14.75 | 9.90 | 166.00 | 5.00 | 1.66 |
| (SEQ ID No.: 2) | | 407.86 | 448.73 | 14.75 | 9.90 | 166.00 | 5.00 | 1.66 |
| | | 407.86 | 410.18 | 14.75 | 9.90 | 166.00 | 5.00 | 1.66 |
| GHMLENHVER(U-13C6, 15N4) | Labelled | 411.20 | 519.26 | 14.75 | 9.90 | 166.00 | 5.00 | 1.66 |
| (SEQ ID No.: 54) | | 411.20 | 453.74 | 14.75 | 9.90 | 166.00 | 5.00 | 1.67 |
| EAQLPVIENK (SEQ ID No.: 3) | Native | 570.82 | 699.40 | 2.12 | 18.70 | 166.00 | 5.00 | 1.67 |
| | | 570.82 | 503.28 | 2.12 | 18.70 | 166.00 | 5.00 | 1.67 |
| | | 570.82 | 812.49 | 2.12 | 18.70 | 166.00 | 5.00 | 1.67 |
| EAQLPVIENK(U-13C6, 15N2) | Labelled | 574.82 | 707.42 | 2.12 | 18.70 | 166.00 | 5.00 | 1.67 |
| (SEQ ID No.: 55) | | 574.82 | 820.50 | 2.12 | 18.70 | 166.00 | 5.00 | 1.95 |
| CQSWSSMTPHR | Native | 459.53 | 510.28 | 3.61 | 11.70 | 166.00 | 5.00 | 1.95 |
| (SEQ ID No.: 4) | | 459.53 | 641.32 | 3.61 | 11.70 | 166.00 | 5.00 | 1.95 |
| | | 459.53 | 409.23 | 3.61 | 11.70 | 166.00 | 5.00 | 1.95 |
| CQSWSSMTPHR | Labelled | 462.87 | 520.29 | 3.61 | 11.70 | 166.00 | 5.00 | 2.09 |
| (U-13C6, 15N4) | | 462.87 | 419.24 | 3.61 | 11.70 | 166.00 | 5.00 | 2.09 |
| (SEQ ID No.: 56) | | | | | | | | |
| EITALAPSTMK | Native | 581.31 | 634.32 | 1.99 | 19.00 | 166.00 | 5.00 | 2.09 |
| (SEQ ID No.: 5) | | 581.31 | 919.49 | 1.99 | 19.00 | 166.00 | 5.00 | 2.09 |
| | | 581.31 | 563.29 | 1.99 | 19.00 | 166.00 | 5.00 | 2.12 |
| EITALAPSTMK(U-13C6, 15N2) | Labelled | 585.32 | 642.34 | 1.99 | 19.00 | 166.00 | 5.00 | 2.12 |
| (SEQ ID No.: 57) | | 585.32 | 927.51 | 1.99 | 19.00 | 166.00 | 5.00 | 2.12 |
| FNAVLTNPQGDYDTSTGK | Native | 964.46 | 1383.60 | 2.03 | 30.90 | 166.00 | 5.00 | 2.12 |
| (SEQ ID No.: 6) | | 964.46 | 1282.55 | 2.03 | 30.90 | 166.00 | 5.00 | 2.12 |
| | | 964.46 | 1168.51 | 2.03 | 30.90 | 166.00 | 5.00 | 2.13 |
| FNAVLTNPQGDYDTSTGK | Labelled | 968.46 | 1290.57 | 2.03 | 30.90 | 166.00 | 5.00 | 2.13 |
| (U-13C6, 15N2) | | 968.46 | 1176.53 | 2.03 | 30.90 | 166.00 | 5.00 | 2.13 |
| (SEQ ID No.: 58) | | | | | | | | |
| TNQVNSGGVLLR | Native | 629.35 | 914.54 | 2.55 | 20.50 | 166.00 | 5.00 | 2.13 |
| (SEQ ID No.: 7) | | 629.35 | 815.47 | 2.55 | 20.50 | 166.00 | 5.00 | 2.23 |
| | | 629.35 | 701.43 | 2.55 | 20.50 | 166.00 | 5.00 | 2.23 |
| TNQVNSGGVLLR | Labelled | 634.35 | 825.48 | 2.55 | 20.50 | 166.00 | 5.00 | 2.23 |
| (U-13C6, 15N4) | | 634.35 | 711.44 | 2.55 | 20.50 | 166.00 | 5.00 | 2.23 |
| (SEQ ID No.: 59) | | | | | | | | |
| ALDFAVGEYNK | Native | 613.81 | 780.39 | 2.07 | 20.00 | 166.00 | 5.00 | 2.82 |
| (SEQ ID No.: 8) | | 613.81 | 709.35 | 2.07 | 20.00 | 166.00 | 5.00 | 2.82 |
| | | | | | | 166.00 | 5.00 | |
| ALDFAVGEYNK | Labelled | 617.81 | 788.40 | 2.07 | 20.00 | 166.00 | 5.00 | 2.82 |
| (U-13C6, 15N2) | | 617.81 | 717.37 | 2.07 | 20.00 | 166.00 | 5.00 | 2.82 |
| (SEQ ID No.: 60) | | | | | | | | |
| LVGGPMDASVEEEGVRR | Native | 600.97 | 745.40 | 2.18 | 16.80 | 166.00 | 5.00 | 2.82 |
| (SEQ ID No.: 9) | | 600.97 | 616.35 | 2.18 | 16.80 | 166.00 | 5.00 | 2.85 |
| | | 600.97 | 737.85 | 2.18 | 16.80 | 166.00 | 5.00 | 2.85 |
| | | 600.97 | 689.32 | 2.18 | 16.80 | 166.00 | 5.00 | 2.85 |
| LVGGPMDASVEEEGVRR | Labelled | 607.64 | 765.41 | 2.18 | 16.80 | 166.00 | 5.00 | 2.85 |
| (U-13C6, 15N4)R | | 607.64 | 636.37 | 2.18 | 16.80 | 166.00 | 5.00 | 2.85 |
| (SEQ ID No.: 61) | | 607.64 | 747.86 | 2.18 | 16.80 | 166.00 | 5.00 | 2.89 |
| | | 607.64 | 699.33 | 2.18 | 16.80 | 166.00 | 5.00 | 2.89 |
| SDVMYTDWK | Native | 572.75 | 942.44 | 1.98 | 18.80 | 166.00 | 5.00 | 2.89 |
| (SEQ ID No.: 10) | | 572.75 | 843.37 | 1.98 | 18.80 | 166.00 | 5.00 | 2.89 |
| | | 572.75 | 712.33 | 1.98 | 18.80 | 166.00 | 5.00 | 2.89 |
| SDVMYTDWK | Labelled | 576.76 | 851.38 | 1.98 | 18.80 | 166.00 | 5.00 | 2.93 |
| (U-13C6, 15N2) | | 576.76 | 720.34 | 1.98 | 18.80 | 166.00 | 5.00 | 2.93 |
| (SEQ ID No.: 62) | | | | | | | | |
| WEMPFDPQDTHQSR | Native | 591.93 | 968.45 | 2.49 | 16.50 | 166.00 | 5.00 | 2.93 |
| (SEQ ID No.: 11) | | 591.93 | 729.83 | 2.49 | 16.50 | 166.00 | 5.00 | 2.93 |
| | | 591.93 | 664.30 | 2.49 | 16.50 | 166.00 | 5.00 | 2.93 |
| WEMPFDPQDTHQSR | Labelled | 595.26 | 734.83 | 2.49 | 16.50 | 166.00 | 5.00 | 2.93 |
| (U-13C6, 15N4) | | 595.26 | 669.31 | 2.49 | 16.50 | 166.00 | 5.00 | 2.93 |
| (SEQ ID No.: 63) | | | | | | | | |
| EQLSLLDR (SEQ ID No.: 12) | Native | 487.27 | 716.43 | 1.99 | 16.10 | 166.00 | 5.00 | 2.93 |
| | | 487.27 | 603.35 | 1.99 | 16.10 | 166.00 | 5.00 | 2.93 |
| EQLSLLDR | Labelled | 492.27 | 726.44 | 1.99 | 16.10 | 166.00 | 5.00 | 2.93 |
| (U-13C6, 15N4) | | 492.27 | 613.35 | 1.99 | 16.10 | 166.00 | 5.00 | 3.01 |
| (SEQ ID No.: 64) | | | | | | | | |
| GDVAFVK (SEQ ID No.: 13) | Native | 368.21 | 563.36 | 3.99 | 12.40 | 166.00 | 5.00 | 3.01 |
| | | 368.21 | 464.29 | 3.99 | 12.40 | 166.00 | 5.00 | 3.01 |
| | | 368.21 | 393.25 | 3.99 | 12.40 | 166.00 | 5.00 | 3.01 |
| | | 368.21 | 246.18 | 3.99 | 12.40 | 166.00 | 5.00 | 3.01 |
| | | 368.21 | 17.11 | 3.99 | 12.40 | 166.00 | 5.00 | 3.02 |
| GDVAFVK | Labelled | 372.21 | 571.37 | 3.99 | 12.40 | 166.00 | 5.00 | 3.02 |
| (U-13C6, 15N2) | | 372.21 | 472.30 | 3.99 | 12.40 | 166.00 | 5.00 | 3.02 |
| (SEQ ID No.: 65) | | 372.21 | 401.26 | 3.99 | 12.40 | 166.00 | 5.00 | 3.02 |
| | | 372.21 | 254.20 | 3.99 | 12.40 | 166.00 | 5.00 | 3.02 |
| | | 372.21 | 155.13 | 3.99 | 12.40 | 166.00 | 5.00 | 3.04 |
| WCALSHHER (SEQ ID No.: 14) | Native | 399.19 | 505.24 | 8.15 | 9.60 | 166.00 | 5.00 | 3.04 |
| | | 399.19 | 425.22 | 8.15 | 9.60 | 166.00 | 5.00 | 3.04 |
| WCALSHHER | Labelled | 402.52 | 510.24 | 8.15 | 9.60 | 166.00 | 5.00 | 3.04 |
| (U-13C6, 15N4) | | 402.52 | 430.22 | 8.15 | 9.60 | 166.00 | 5.00 | 3.04 |
| (SEQ ID No.: 66) | | | | | | | | |
| IAELSATAQEIIK | Native | 693.90 | 960.54 | 3.43 | 22.50 | 166.00 | 5.00 | 3.28 |
| (SEQ ID No.: 15) | | 693.90 | 873.50 | 3.43 | 22.50 | 166.00 | 5.00 | 3.28 |
| | | 693.90 | 802.47 | 3.43 | 22.50 | 166.00 | 5.00 | 3.28 |
| IAELSATAQEIIK | Labelled | 697.90 | 968.55 | 3.43 | 22.50 | 166.00 | 5.00 | 3.28 |
| (U-13C6, 15N2) | | 697.90 | 881.52 | 3.43 | 22.50 | 166.00 | 5.00 | 3.28 |
| (SEQ ID No.: 67) | | 697.90 | 810.48 | 3.43 | 22.50 | 166.00 | 5.00 | 3.39 |
| EQHLFLPFSYK | Native | 470.25 | 641.33 | 7.21 | 12.10 | 166.00 | 5.00 | 3.39 |
| (SEQ ID No.: 16) | | 470.25 | 544.28 | 7.21 | 12.10 | 166.00 | 5.00 | 3.39 |
| | | 470.25 | 655.32 | 7.21 | 12.10 | 166.00 | 5.00 | 3.39 |
| EQHLFLPFSYK | Labelled | 472.92 | 649.34 | 7.21 | 12.10 | 166.00 | 5.00 | 3.39 |
| (U-13C6, 15N2) | | 472.92 | 552.29 | 7.21 | 12.10 | 166.00 | 5.00 | 3.39 |
| (SEQ ID No.: 68) | | | | | | | | |
| ADQVCINLR (SEQ ID No.: 17) | Native | 544.78 | 774.43 | 2.12 | 17.90 | 166.00 | 5.00 | 3.50 |
| | | 544.78 | 675.36 | 2.12 | 17.90 | 166.00 | 5.00 | 3.50 |
| | | 544.78 | 402.25 | 2.12 | 17.90 | 166.00 | 5.00 | 3.50 |
| ADQVCINLR | Labelled | 549.78 | 784.44 | 2.12 | 17.90 | 166.00 | 5.00 | 3.50 |
| (U-13C6, 15N4) | | 549.78 | 685.37 | 2.12 | 17.90 | 166.00 | 5.00 | 3.50 |
| (SEQ ID No.: 69) | | | | | | | | |
| ILTSDVFQDCNK | Native | 720.35 | 1112.47 | 2.09 | 23.30 | 166.00 | 5.00 | 3.52 |
| (SEQ ID No.: 18) | | 720.35 | 1025.44 | 2.09 | 23.30 | 166.00 | 5.00 | 3.52 |
| | | 720.35 | 664.27 | 2.09 | 23.30 | 166.00 | 5.00 | 3.52 |
| ILTSDVFQDCNK | Labelled | 724.35 | 1120.48 | 2.09 | 23.30 | 166.00 | 5.00 | 3.52 |
| (U-13C6, 15N2) | | 724.35 | 1033.45 | 2.09 | 23.30 | 166.00 | 5.00 | 3.52 |
| (SEQ ID No.: 70) | | | | | | | | |
| YAGSQVASTSEVLK | Native | 720.37 | 933.53 | 2.38 | 23.30 | 166.00 | 5.00 | 3.54 |
| (SEQ ID No.: 19) | | 720.37 | 834.46 | 2.38 | 23.30 | 166.00 | 5.00 | 3.54 |
| | | 720.37 | 763.42 | 2.38 | 23.30 | 166.00 | 5.00 | 3.54 |
| YAGSQVASTSEVLK | Labelled | 724.38 | 941.54 | 2.38 | 23.30 | 166.00 | 5.00 | 3.54 |
| (U-13C6, 15N2) | | 724.38 | 842.47 | 2.38 | 23.30 | 166.00 | 5.00 | 3.54 |
| (SEQ ID No.: 71) | | 724.38 | 771.43 | 2.38 | 23.30 | 166.00 | 5.00 | 3.56 |
| ESDTSYVSLK | Native | 564.77 | 696.39 | 3.38 | 18.50 | 166.00 | 5.00 | 3.56 |
| (SEQ ID No.: 20) | | 564.77 | 609.36 | 3.38 | 18.50 | 166.00 | 5.00 | 3.56 |
| | | 564.77 | 446.30 | 3.38 | 18.50 | 166.00 | 5.00 | 3.56 |
| ESDTSYVSLK | Labelled | 568.78 | 617.37 | 3.38 | 18.50 | 166.00 | 5.00 | 3.56 |
| (U-13C6, 15N2) | | 568.78 | 454.31 | 3.38 | 18.50 | 166.00 | 5.00 | 3.59 |
| (SEQ ID No.: 72) | | | | | | | | |
| GYSIFSYATK | Native | 568.78 | 916.48 | 2.49 | 18.60 | 166.00 | 5.00 | 3.59 |
| (SEQ ID No.: 21) | | 568.78 | 829.45 | 2.49 | 18.60 | 166.00 | 5.00 | 3.59 |
| | | 568.78 | 716.36 | 2.49 | 18.60 | 166.00 | 5.00 | 3.59 |
| GYSIFSYATK | Labelled | 572.79 | 924.49 | 2.49 | 18.60 | 166.00 | 5.00 | 3.59 |
| (U-13C6, 15N2) | | 572.79 | 724.38 | 2.49 | 18.60 | 166.00 | 5.00 | 3.59 |
| (SEQ ID No.: 73) | | | | | | | | |
| STDYGIFQINSR | Native | 700.84 | 934.51 | 2.72 | 22.70 | 166.00 | 5.00 | 3.59 |
| (SEQ ID No. 22) | | 700.84 | 764.40 | 2.72 | 22.70 | 166.00 | 5.00 | 3.59 |
| | | 700.84 | 617.34 | 2.72 | 22.70 | 166.00 | 5.00 | 3.69 |
| STDYGIFQINSR | Labelled | 705.85 | 944.52 | 2.72 | 22.70 | 166.00 | 5.00 | 3.69 |
| (U-13C6, 15N4) | | 705.85 | 774.41 | 2.72 | 22.70 | 166.00 | 5.00 | 3.69 |
| (SEQ ID No.: 74) | | 705.85 | 627.34 | 2.72 | 22.70 | 166.00 | 5.00 | 3.69 |
| YWCNDGK (SEQ ID No.: 23) | Native | 471.69 | 779.31 | 11.56 | 15.60 | 166.00 | 5.00 | 3.69 |
| | | 471.69 | 593.23 | 11.56 | 15.60 | 166.00 | 5.00 | 3.69 |
| YWCNDGK | Labelled | 475.70 | 787.33 | 11.56 | 15.60 | 166.00 | 5.00 | 3.69 |
| (U-13C6, 15N2) | | 475.70 | 601.25 | 11.56 | 15.60 | 166.00 | 5.00 | 3.69 |
| (SEQ ID No.: 75) | | | | | | | | |
| AHVDALR (SEQ ID No.: 24) | Native | 261.15 | 288.20 | 21.50 | 4.60 | 166.00 | 5.00 | 3.69 |
| | | 261.15 | 423.20 | 21.50 | 4.60 | 166.00 | 5.00 | 3.69 |
| AHVDALR | Labelled | 264.48 | 298.21 | 21.50 | 4.60 | 166.00 | 5.00 | 3.82 |
| (U-13C6, 15N4) | | 264.48 | 423.20 | 21.50 | 4.60 | 166.00 | 5.00 | 3.82 |
| (SEQ ID No.: 76) | | | | | | | | |
| ATEHLSTLSEK | Native | 405.88 | 522.27 | 6.70 | 9.80 | 166.00 | 5.00 | 3.82 |
| (SEQ ID No.: 25) | | 405.88 | 457.75 | 6.70 | 9.80 | 166.00 | 5.00 | 3.82 |
| ATEHLSTLSEK | Labelled | 408.55 | 526.28 | 6.70 | 9.80 | 166.00 | 5.00 | 3.82 |
| (U-13C6, 15N2) | | 408.55 | 461.76 | 6.70 | 9.80 | 166.00 | 5.00 | 3.85 |
| (SEQ ID No.: 77) | | | | | | | | |
| TVVQPSVGAAAGPVVPPCPGR | Native | 672.69 | 683.33 | 2.02 | 19.40 | 166.00 | 5.00 | 3.85 |
| (SEQ ID No.: 26) | | 672.69 | 489.76 | 2.02 | 19.40 | 166.00 | 5.00 | 3.85 |
| | | 672.69 | 1038.56 | 2.02 | 19.40 | 166.00 | 5.00 | 3.85 |
| TVVQPSVGAAAGPVVPPCPGR | Labelled | 676.03 | 693.34 | 2.02 | 19.40 | 166.00 | 5.00 | 3.95 |
| (U-13C6, 15N4) | | 676.03 | 494.77 | 2.02 | 19.40 | 166.00 | 5.00 | 3.95 |
| (SEQ ID No.: 78) | | | | | | | | |
| CNLLAEK (SEQ ID No.: 27) | Native | 424.22 | 573.36 | 5.01 | 14.20 | 166.00 | 5.00 | 3.95 |
| | | 424.22 | 460.28 | 5.01 | 14.20 | 166.00 | 5.00 | 3.95 |
| | | 424.22 | 347.19 | 5.01 | 14.20 | 166.00 | 5.00 | 3.95 |
| CNLLAEK | Labelled | 428.23 | 581.37 | 5.01 | 14.20 | 166.00 | 5.00 | 4.01 |
| (U-13C6, 15N2) | | 428.23 | 355.21 | 5.01 | 14.20 | 166.00 | 5.00 | 4.01 |
| (SEQ ID No.: 79) | | | | | | | | |
| GGEGTGYFVDFSVR | Native | 497.57 | 623.31 | 5.59 | 13.10 | 166.00 | 5.00 | 4.01 |
| (SEQ ID No.: 28) | | 497.57 | 508.29 | 5.59 | 13.10 | 166.00 | 5.00 | 4.01 |
| | | 497.57 | 624.31 | 5.59 | 13.10 | 166.00 | 5.00 | 4.01 |
| GGEGTGYFVDFSVR | Labelled | 500.90 | 518.30 | 5.59 | 13.10 | 166.00 | 5.00 | 4.08 |
| (U-13C6, 15N4) | | 500.90 | 629.31 | 5.59 | 13.10 | 166.00 | 5.00 | 4.08 |
| (SEQ ID No.: 80) | | | | | | | | |
| IADAHLDR (SEQ ID No.: 29) | Native | 304.16 | 403.23 | 9.24 | 6.10 | 166.00 | 5.00 | 4.08 |
| | | 304.16 | 399.20 | 9.24 | 6.10 | 166.00 | 5.00 | 4.08 |
| | | 304.16 | 363.68 | 9.24 | 6.10 | 166.00 | 5.00 | 4.08 |
| IADAHLDR | Labelled | 307.50 | 404.20 | 9.24 | 6.10 | 166.00 | 5.00 | 4.15 |
| (U-13C6, 15N4) | | 307.50 | 368.68 | 9.24 | 6.10 | 166.00 | 5.00 | 4.15 |
| (SEQ ID No.: 81) | | | | | | | | |
| GCPDVQASLPDAK | Native | 679.32 | 1140.59 | 2.40 | 22.10 | 166.00 | 5.00 | 4.15 |
| (SEQ ID No.: 32) | | 679.32 | 430.23 | 2.40 | 22.10 | 166.00 | 5.00 | 4.15 |
| | | 679.32 | 570.80 | 2.40 | 22.10 | 166.00 | 5.00 | 4.15 |
| GCPDVQASLPDAK | Labelled | 683.33 | 1148.60 | 2.40 | 22.10 | 166.00 | 5.00 | 4.18 |
| (U-13C6, 15N2) | | 683.33 | 574.81 | 2.40 | 22.10 | 166.00 | 5.00 | 4.18 |
| (SEQ ID No.: 84) | | | | 2.40 | 22.10 | | | |
| TFTLLDPK (SEQ ID No.: 33) | Native | 467.77 | 686.41 | 2.32 | 15.50 | 166.00 | 5.00 | 4.18 |
| | | 467.77 | 585.36 | 2.32 | 15.50 | 166.00 | 5.00 | 4.18 |
| | | 467.77 | 472.28 | 2.32 | 15.50 | 166.00 | 5.00 | 4.18 |
| TFTLLDPK | Labelled | 471.77 | 694.42 | 2.32 | 15.50 | 166.00 | 5.00 | 4.19 |
| (U-13C6, 15N2) | | 471.77 | 593.37 | 2.32 | 15.50 | 166.00 | 5.00 | 4.19 |
| (SEQ ID No.: 85) | | | | | | | | |
| GSPAINVAVHVFR | Native | 456.26 | 558.31 | 2.80 | 11.60 | 166.00 | 5.00 | 4.19 |
| (SEQ ID No.: 34) | | 456.26 | 563.33 | 2.80 | 11.60 | 166.00 | 5.00 | 4.19 |
| | | 456.26 | 527.81 | 2.80 | 11.60 | 166.00 | 5.00 | 4.19 |
| GSPAINVAVHVFR | Labelled | 459.59 | 568.33 | 2.80 | 11.60 | 166.00 | 5.00 | 4.19 |
| (U-13C6, 15N4) | | 459.59 | 532.82 | 2.80 | 11.60 | 166.00 | 5.00 | 4.19 |
| (SEQ ID No.: 86) | | | | | | | | |
| AADDTWEPFASGK | Native | 697.81 | 921.45 | 2.02 | 22.60 | 166.00 | 5.00 | 4.19 |
| (SEQ ID No.: 35) | | 697.81 | 735.37 | 2.02 | 22.60 | 166.00 | 5.00 | 4.30 |
| AADDTWEPFASGK | Labelled | 701.82 | 929.46 | 2.02 | 22.60 | 166.00 | 5.00 | 4.30 |
| (U-13C6, 15N2) | | 701.82 | 614.34 | 2.02 | 22.60 | 166.00 | 5.00 | 4.30 |
| (SEQ ID No.: 87) | | | | | | | | |
| DSVTGTLPK (SEQ ID No.: 36) | Native | 459.25 | 715.43 | 4.31 | 15.20 | 166.00 | 5.00 | 4.43 |
| | | 459.25 | 616.37 | 4.31 | 15.20 | 166.00 | 5.00 | 4.43 |
| | | 459.25 | 515.32 | 4.31 | 15.20 | 166.00 | 5.00 | 4.43 |
| DSVTGTLPK | Labelled | 463.26 | 624.38 | 4.31 | 15.20 | 166.00 | 5.00 | 4.43 |
| (U-13C6, 15N2) | | 463.26 | 523.33 | 4.31 | 15.20 | 166.00 | 5.00 | 4.43 |
| (SEQ ID No.: 88) | | | | | | | | |
| IAYGTQGSSGYSLR | Native | 730.36 | 954.46 | 2.60 | 23.60 | 166.00 | 5.00 | 4.51 |
| (SEQ ID No.: 37) | | 730.36 | 826.41 | 2.60 | 23.60 | 166.00 | 5.00 | 4.51 |
| | | 730.36 | 682.34 | 2.60 | 23.60 | 166.00 | 5.00 | 4.51 |
| IAYGTQGSSGYSLR | Labelled | 735.37 | 964.47 | 2.60 | 23.60 | 166.00 | 5.00 | 4.51 |
| (U-13C6, 15N4) | | 735.37 | 692.36 | 2.60 | 23.60 | 166.00 | 5.00 | 4.51 |
| (SEQ ID No.: 89) | | | | | | | | |
| ANRPFLVFIR (SEQ ID No.: 38) | Native | 411.58 | 534.34 | 5.80 | 10.00 | 166.00 | 5.00 | 4.51 |
| | | 411.58 | 435.27 | 5.80 | 10.00 | 166.00 | 5.00 | 4.51 |
| ANRPFLVFIR | Labelled | 414.92 | 544.35 | 5.80 | 10.00 | 166.00 | 5.00 | 4.51 |
| (U-13C6, 15N4) | | 414.92 | 445.28 | 5.80 | 10.00 | 166.00 | 5.00 | 4.51 |
| (SEQ ID No.: 90) | | | | | | | | |
| TSCLLFMGR (SEQ ID No.: 39) | Native | 542.77 | 896.45 | 2.90 | 17.80 | 166.00 | 5.00 | 4.51 |
| | | 542.77 | 736.42 | 2.90 | 17.80 | 166.00 | 5.00 | 4.52 |
| | | 542.77 | 623.33 | 2.90 | 17.80 | 166.00 | 5.00 | 4.52 |
| TSCLLFMGR | Labelled | 547.77 | 906.46 | 2.90 | 17.80 | 166.00 | 5.00 | 4.52 |
| (U-13C6, 15N4) | | 547.77 | 633.34 | 2.90 | 17.80 | 166.00 | 5.00 | 4.52 |
| (SEQ ID No.: 91) | | | | | | | | |
| HFQNLGK (SEQ ID No.: 40) | Native | 281.82 | 317.22 | 13.01 | 5.30 | 166.00 | 5.00 | 4.52 |
| | | 281.82 | 413.19 | 13.01 | 5.30 | 166.00 | 5.00 | 4.54 |
| | | 281.82 | 527.24 | 13.01 | 5.30 | 166.00 | 5.00 | 4.54 |
| HFQNLGK | Labelled | 284.49 | 413.19 | 13.01 | 5.30 | 166.00 | 5.00 | 4.54 |
| (U-13C6, 15N2) | | 284.49 | 527.24 | 13.01 | 5.30 | 166.00 | 5.00 | 4.54 |
| (SEQ ID No.: 92) | | | | | | | | |
| TINPAVDHCCK | Native | 438.87 | 550.73 | 7.32 | 11.00 | 166.00 | 5.00 | 4.54 |
| (SEQ ID No.: 41) | | 438.87 | 493.71 | 7.32 | 11.00 | 166.00 | 5.00 | 4.54 |
| TINPAVDHCCK | Labelled | 441.54 | 554.74 | 7.32 | 11.00 | 166.00 | 5.00 | 4.63 |
| (U-13C6, 15N2) | | 441.54 | 497.72 | 7.32 | 11.00 | 166.00 | 5.00 | 4.63 |
| (SEQ ID No.: 93) | | | | | | | | |
| LLDSLPSDTR (SEQ ID No.: 42) | Native | 558.80 | 775.39 | 2.06 | 18.30 | 166.00 | 5.00 | 4.63 |
| | | 558.80 | 575.28 | 2.06 | 18.30 | 166.00 | 5.00 | 4.63 |
| | | 558.80 | 542.32 | 2.06 | 18.30 | 166.00 | 5.00 | 4.63 |
| LLDSLPSDTR | Labelled | 563.80 | 785.40 | 2.06 | 18.30 | 166.00 | 5.00 | 4.63 |
| (U-13C6, 15N4) | | 563.80 | 585.29 | 2.06 | 18.30 | 166.00 | 5.00 | 4.64 |
| (SEQ ID No.: 94) | | | | | | | | |
| LVLLNAIYLSAK | Native | 659.41 | 879.49 | 17.14 | 21.40 | 166.00 | 5.00 | 4.64 |
| (SEQ ID No.: 43) | | 659.41 | 694.41 | 17.14 | 21.40 | 166.00 | 5.00 | 4.64 |
| | | 659.41 | 581.33 | 17.14 | 21.40 | 166.00 | 5.00 | 4.64 |
| LVLLNAIYLSAK | Labelled | 663.42 | 887.51 | 17.14 | 21.40 | 166.00 | 5.00 | 4.64 |
| (U-13C6, 15N2) | | 663.42 | 589.34 | 17.14 | 21.40 | 166.00 | 5.00 | 4.91 |
| (SEQ ID No.: 95) | | | | | | | | |
| ILNIFGVIK (SEQ ID No.: 44) | Native | 508.83 | 790.48 | 29.95 | 16.80 | 166.00 | 5.00 | 4.91 |
| | | 508.83 | 676.44 | 29.95 | 16.80 | 166.00 | 5.00 | 4.91 |
| | | 508.83 | 563.36 | 29.95 | 16.80 | 166.00 | 5.00 | 4.91 |
| ILNIFGVIK | Labelled | 512.84 | 798.50 | 29.95 | 16.80 | 166.00 | 5.00 | 4.91 |
| (U-13C6, 15N2) | | 512.84 | 571.37 | 29.95 | 16.80 | 166.00 | 5.00 | 4.97 |
| (SEQ ID No.: 96) | | | | | | | | |
| VSASPLLYTLIEK | Native | 717.42 | 1089.66 | 10.80 | 23.20 | 166.00 | 5.00 | 4.97 |
| (SEQ ID No.: 45) | | 717.42 | 879.52 | 10.80 | 23.20 | 166.00 | 5.00 | 4.97 |
| | | 717.42 | 545.33 | 10.80 | 23.20 | 166.00 | 5.00 | 4.97 |
| VSASPLLYTLIEK | Labelled | 721.42 | 887.53 | 10.80 | 23.20 | 166.00 | 5.00 | 4.97 |
| (U-13C6, 15N2) | | 721.42 | 549.34 | 10.80 | 23.20 | 166.00 | 5.00 | 5.01 |
| (SEQ ID No.: 97) | | | | | | | | |
| DSGSYFCR (SEQ ID No.: 46) | Native | 496.20 | 789.33 | 5.56 | 16.40 | 166.00 | 5.00 | 5.01 |
| | | 496.20 | 732.31 | 5.56 | 16.40 | 166.00 | 5.00 | 5.01 |
| | | 496.20 | 645.28 | 5.56 | 16.40 | 166.00 | 5.00 | 5.01 |
| DSGSYFCR | Labelled | 501.20 | 799.34 | 5.56 | 16.40 | 166.00 | 5.00 | 5.03 |
| (U-13C6, 15N4) | | 501.20 | 655.29 | 5.56 | 16.40 | 166.00 | 5.00 | 5.03 |
| (SEQ ID No.: 98) | | | | | | | | |
| VLDLSCNR (SEQ D No.: 47) | Native | 488.75 | 877.42 | 3.68 | 16.20 | 166.00 | 5.00 | 5.03 |
| | | 488.75 | 764.34 | 3.68 | 16.20 | 166.00 | 5.00 | 5.03 |
| | | 488.75 | 649.31 | 3.68 | 16.20 | 166.00 | 5.00 | 5.03 |
| VLDLSCNR | Labelled | 493.75 | 774.34 | 3.68 | 16.20 | 166.00 | 5.00 | 5.20 |
| (U-13C6, 15N4) | | 493.75 | 659.32 | 3.68 | 16.20 | 166.00 | 5.00 | 5.20 |
| (SEQ ID No.: 99) | | | | | | | | |
| DFALQNPSAVPR | Native | 657.84 | 981.55 | 2.07 | 21.40 | 166.00 | 5.00 | 5.20 |
| (SEQ ID No.: 48) | | 657.84 | 740.40 | 2.07 | 21.40 | 166.00 | 5.00 | 5.20 |
| | | 657.84 | 626.36 | 2.07 | 21.40 | 166.00 | 5.00 | 5.20 |
| DFALQNPSAVPR | Labelled | 662.85 | 750.41 | 2.07 | 21.40 | 166.00 | 5.00 | 5.61 |
| (U-13C6, 15N4) | | 662.85 | 636.37 | 2.07 | 21.40 | 166.00 | 5.00 | 5.61 |
| (SEQ ID No.: 100) | | | | | | | | |
| LAELPADALGPLQR | Native | 732.41 | 1037.57 | 6.28 | 23.70 | 166.00 | 5.00 | 5.61 |
| (SEQ ID No.: 49) | | 732.41 | 570.34 | 6.28 | 23.70 | 166.00 | 5.00 | 5.61 |
| | | 732.41 | 519.29 | 6.28 | 23.70 | 166.00 | 5.00 | 5.61 |
| LAELPADALGPLQR | Labelled | 737.42 | 1047.58 | 6.28 | 23.70 | 166.00 | 5.00 | 5.61 |
| (U-13C6, 15N4) | | 737.42 | 524.29 | 6.28 | 23.70 | 166.00 | 5.00 | 5.84 |
| (SEQ ID No.: 101) | | | | | | | | |
| ASDTAMYYCAR | Native | 654.77 | 934.39 | 2.14 | 21.30 | 166.00 | 5.00 | 5.84 |
| (SEQ ID No.: 50) | | 654.77 | 863.35 | 2.14 | 21.30 | 166.00 | 5.00 | 5.84 |
| | | 654.77 | 732.31 | 2.14 | 21.30 | 166.00 | 5.00 | 5.84 |
| | | 654.77 | 569.25 | 2.14 | 21.30 | 166.00 | 5.00 | 5.84 |
| ASDTAMYYCAR | Labelled | 659.78 | 944.40 | 2.14 | 21.30 | 166.00 | 5.00 | 5.88 |
| (U-13C6, 15N4) | | 659.78 | 873.36 | 2.14 | 21.30 | 166.00 | 5.00 | 5.88 |
| (SEQ ID No.: 102) | | 659.78 | 742.32 | 2.14 | 21.30 | 166.00 | 5.00 | 5.88 |
| | | 659.78 | 579.26 | 2.14 | 21.30 | 166.00 | 5.00 | 5.88 |
| VEGTAFVIFGIQDGEQR | Native | 622.65 | 732.33 | 9.89 | 17.60 | 166.00 | 5.00 | 5.88 |
| (SEQ ID No.: 51) | | 622.65 | 604.27 | 9.89 | 17.60 | 166.00 | 5.00 | 5.91 |
| | | 622.65 | 489.24 | 9.89 | 17.60 | 166.00 | 5.00 | 5.91 |
| VEGTAFVIFGIQDGEQR | Labelled | 625.99 | 742.34 | 9.89 | 17.60 | 166.00 | 5.00 | 5.91 |
| (U-13C6, 15N4) | | 625.99 | 614.28 | 9.89 | 17.60 | 166.00 | 5.00 | 5.91 |
| (SEQ ID No.: 103) | | 625.99 | 499.25 | 9.89 | 17.60 | 166.00 | 5.00 | 5.91 |
| VHQYFNVELIQPGAVK | Native | 614.67 | 825.52 | 5.42 | 17.30 | 166.00 | 5.00 | 5.99 |
| (SEQ ID No.: 52) | | 614.67 | 712.44 | 5.42 | 17.30 | 166.00 | 5.00 | 5.99 |
| | | 614.67 | 471.29 | 5.42 | 17.30 | 166.00 | 5.00 | 5.99 |
| VHQYFNVELIQPGAVK | Labelled | 617.34 | 720.45 | 5.42 | 17.30 | 166.00 | 5.00 | 5.99 |
| (U-13C6, 15N2) | | 617.34 | 479.31 | 5.42 | 17.30 | 166.00 | 5.00 | 5.99 |
| (SEQ ID No.: 104) | | | | | | | | |

Table 7 shows native and labelled peptide MRM transitions on 7500 (Sciex) LC-MS/MS platform. Internal standard (labelled) peptide sequences are shown in their post-digestion form (without tryptic tags). The following abbreviations are used in table 7: EP—entrance potential; CE—collision energy; CXP—collision cell exit potential; DP—declustering potential; RT—retention time

TABLE 7

| Peptide | | | Product | | | | | |
|---|---|---|---|---|---|---|---|---|
| Standard | Standard | Precursor | (Q3) | Dwell | | | | RT |
| Sequence | type | (Q1) m/z | m/z | time (ms) | EP | CE | CXP | (min) |
| GLPNVVTSAISLPNIR | Native | 825.98 | 1170.68 | 3.50 | 10.00 | 43.30 | 25.00 | 6.58 |
| (SEQ ID No.: 1) | | 825.98 | 1071.62 | 3.50 | 10.00 | 43.30 | 25.00 | 6.58 |
| | | 825.98 | 499.30 | 3.50 | 10.00 | 31.30 | 25.00 | 6.58 |
| GLPNVVTSAISLPNIR | Labelled | 830.98 | 1180.69 | 3.50 | 10.00 | 43.30 | 25.00 | 6.58 |
| (U-13C6, 15N4) (SEQ ID No.: 53) | | 830.98 | 1081.62 | 3.50 | 10.00 | 43.30 | 25.00 | 6.58 |
| GHMLENHVER (SEQ ID No.: 2) | Native | 407.86 | 783.37 | 3.50 | 10.00 | 20.20 | 25.00 | 2.90 |
| | | 407.86 | 654.33 | 3.50 | 10.00 | 24.20 | 25.00 | 2.90 |
| | | 407.86 | 403.23 | 3.50 | 10.00 | 24.20 | 25.00 | 2.90 |
| GHMLENHVER(U-13C6, 15N4) | Labelled | 411.20 | 793.38 | 3.50 | 10.00 | 20.20 | 25.00 | 2.90 |
| (SEQ ID No.: 54) | | 411.20 | 413.23 | 3.50 | 10.00 | 24.20 | 25.00 | 2.90 |
| EAQLPVIENK (SEQ ID No.: 3) | Native | 570.82 | 699.40 | 3.50 | 10.00 | 26.50 | 25.00 | 4.38 |
| | | 570.82 | 503.28 | 3.50 | 10.00 | 34.50 | 25.00 | 4.38 |
| | | 570.82 | 390.20 | 3.50 | 10.00 | 34.50 | 25.00 | 4.38 |
| EAQLPVIENK(U-13C6, 15N2) | Labelled | 574.84 | 707.41 | 3.50 | 10.00 | 26.50 | 25.00 | 4.38 |
| (SEQ ID No.: 55) | | 574.84 | 511.30 | 3.50 | 10.00 | 34.50 | 25.00 | 4.38 |
| CQSWSSMTPHR (SEQ ID No.: 4) | Native | 459.53 | 815.38 | 3.50 | 10.00 | 22.60 | 25.00 | 3.81 |
| | | 459.53 | 510.28 | 3.50 | 10.00 | 22.60 | 25.00 | 3.81 |
| | | 459.53 | 409.23 | 3.50 | 10.00 | 22.60 | 25.00 | 3.81 |
| CQSWSSMTPHR(U-13C6, 15N4) | Labelled | 462.86 | 520.28 | 3.50 | 10.00 | 22.60 | 25.00 | 3.81 |
| (SEQ ID No.: 56) | | 462.86 | 419.23 | 3.50 | 10.00 | 22.60 | 25.00 | 3.81 |
| EITALAPSTMK (SEQ ID No.: 5) | Native | 581.31 | 747.41 | 3.50 | 10.00 | 27.10 | 25.00 | 4.49 |
| | | 581.31 | 634.32 | 3.50 | 10.00 | 23.10 | 25.00 | 4.49 |
| | | 581.31 | 563.29 | 3.50 | 10.00 | 23.10 | 25.00 | 4.49 |
| EITALAPSTMK(U-13C6, 15N2) | Labelled | 585.31 | 755.42 | 3.50 | 10.00 | 27.10 | 25.00 | 4.49 |
| (SEQ ID No.: 57) | | 585.31 | 642.33 | 3.50 | 10.00 | 23.10 | 25.00 | 4.49 |
| FNAVLTNPQGDYDTSTGK | Native | 643.31 | 1168.51 | 3.50 | 10.00 | 46.20 | 25.00 | 4.67 |
| (SEQ ID No.: 6) | | 643.31 | 584.76 | 3.50 | 10.00 | 54.20 | 25.00 | 4.67 |
| | | 643.31 | 493.26 | 3.50 | 10.00 | 58.20 | 25.00 | 4.67 |
| FNAVLTNPQGDYDTSTGK | Labelled | 645.97 | 1176.52 | 3.50 | 10.00 | 46.20 | 25.00 | 4.67 |
| (U-13C6, 15N2) | | 645.97 | 588.76 | 3.50 | 10.00 | 54.20 | 25.00 | 4.67 |
| (SEQ ID No.: 58) | | | | | | | | |
| TNQVNSGGVLLR | Native | 629.35 | 914.54 | 3.50 | 10.00 | 33.50 | 25.00 | 4.16 |
| (SEQ ID No.: 7) | | 629.35 | 815.47 | 3.50 | 10.00 | 33.50 | 25.00 | 4.16 |
| | | 629.35 | 701.43 | 3.50 | 10.00 | 33.50 | 25.00 | 4.16 |
| TNQVNSGGVLLR(U-13C6, 15N4) | Labelled | 634.35 | 924.55 | 3.50 | 10.00 | 33.50 | 25.00 | 4.16 |
| (SEQ ID No.: 59) | | 634.35 | 825.48 | 3.50 | 10.00 | 33.50 | 25.00 | 4.16 |
| ALDFAVGEYNK | Native | 613.81 | 780.39 | 3.50 | 10.00 | 28.70 | 25.00 | 5.05 |
| (SEQ ID No.: 8) | | 613.81 | 709.35 | 3.50 | 10.00 | 28.70 | 25.00 | 5.05 |
| | | 613.81 | 610.28 | 3.50 | 10.00 | 24.70 | 25.00 | 5.05 |
| ALDFAVGEYNK(U-13C6, 15N2) | Labelled | 617.81 | 717.36 | 3.50 | 10.00 | 28.70 | 25.00 | 5.05 |
| (SEQ ID No.: 60) | | 617.81 | 618.29 | 3.50 | 10.00 | 24.70 | 25.00 | 5.05 |
| LVGGPMDASVEEEGVRR | Native | 600.97 | 745.40 | 3.50 | 10.00 | 37.20 | 25.00 | 4.38 |
| (SEQ ID No.: 9) | | 600.97 | 794.87 | 3.50 | 10.00 | 25.20 | 25.00 | 4.38 |
| | | 600.97 | 566.29 | 3.50 | 10.00 | 37.20 | 25.00 | 4.38 |
| LVGGPMDASVEEEGVRR | Labelled | 604.30 | 799.88 | 3.50 | 10.00 | 25.20 | 25.00 | 4.38 |
| (U-13C6, 15N4)R | | 604.30 | 571.30 | 3.50 | 10.00 | 37.20 | 25.00 | 4.38 |
| (SEQ ID No.: 61) | | | | | | | | |
| SDVMYTDWK (SEQ ID No.: 10) | Native | 572.75 | 843.37 | 3.50 | 10.00 | 26.60 | 25.00 | 4.65 |
| | | 572.75 | 712.33 | 3.50 | 10.00 | 26.60 | 25.00 | 4.65 |
| | | 572.75 | 549.27 | 3.50 | 10.00 | 26.60 | 25.00 | 4.65 |
| SDVMYTDWK | Labelled | 576.75 | 851.38 | 3.50 | 10.00 | 26.60 | 25.00 | 4.65 |
| (U-13C6, 15N2) | | 576.75 | 720.34 | 3.50 | 10.00 | 26.60 | 25.00 | 4.65 |
| (SEQ ID No.: 62) | | | | | | | | |
| WEMPFDPQDTHQSR | Native | 591.93 | 968.45 | 3.50 | 10.00 | 32.80 | 25.00 | 5.34 |
| (SEQ ID No.: 11) | | 591.93 | 628.32 | 3.50 | 10.00 | 40.80 | 25.00 | 5.34 |
| | | 591.93 | 729.83 | 3.50 | 10.00 | 24.80 | 25.00 | 5.34 |
| WEMPFDPQDTHQSR | Labelled | 595.26 | 978.46 | 3.50 | 10.00 | 32.80 | 25.00 | 5.34 |
| (U-13C6, 15N4) | | 595.26 | 734.82 | 3.50 | 10.00 | 24.80 | 25.00 | 5.34 |
| (SEQ ID No.: 63) | | | | | | | | |
| EQLSLLDR (SEQ ID No.: 12) | Native | 487.27 | 716.43 | 3.50 | 10.00 | 22.40 | 25.00 | 4.69 |
| | | 487.27 | 603.35 | 3.50 | 10.00 | 22.40 | 25.00 | 4.69 |
| | | 487.27 | 403.23 | 3.50 | 10.00 | 22.40 | 25.00 | 4.69 |
| EQLSLLDR | Labelled | 492.27 | 726.43 | 3.50 | 10.00 | 22.40 | 25.00 | 4.69 |
| (U-13C6, 15N4) | | 492.27 | 613.35 | 3.50 | 10.00 | 22.40 | 25.00 | 4.69 |
| (SEQ ID No.: 64) | | | | | | | | |
| GDVAFVK (SEQ ID No.: 13) | Native | 368.21 | 563.36 | 3.50 | 10.00 | 16.40 | 25.00 | 3.69 |
| | | 368.21 | 464.29 | 3.50 | 10.00 | 16.40 | 25.00 | 3.69 |
| | | 368.21 | 393.25 | 3.50 | 10.00 | 20.40 | 25.00 | 3.69 |
| GDVAFVK | Labelled | 372.21 | 571.36 | 3.50 | 10.00 | 16.40 | 25.00 | 3.69 |
| (U-13C6, 15N2) | | 372.21 | 472.30 | 3.50 | 10.00 | 16.40 | 25.00 | 3.69 |
| (SEQ ID No.: 65) | | | | | | | | |
| WCALSHHER (SEQ ID No.: 14) | Native | 399.19 | 665.31 | 3.50 | 10.00 | 23.80 | 25.00 | 3.16 |
| | | 399.19 | 578.28 | 3.50 | 10.00 | 27.80 | 25.00 | 3.16 |
| | | 399.19 | 441.22 | 3.50 | 10.00 | 27.80 | 25.00 | 3.16 |
| WCALSHHER | Labelled | 402.52 | 675.31 | 3.50 | 10.00 | 23.80 | 25.00 | 3.16 |
| (U-13C6, 15N4) | | 402.52 | 451.22 | 3.50 | 10.00 | 27.80 | 25.00 | 3.16 |
| (SEQ ID No.: 66) | | | | | | | | |
| IAELSATAQEIIK | Native | 693.90 | 960.54 | 3.50 | 10.00 | 32.70 | 25.00 | 5.32 |
| (SEQ ID No.: 15) | | 693.90 | 873.50 | 3.50 | 10.00 | 32.70 | 25.00 | 5.32 |
| | | 693.90 | 802.47 | 3.50 | 10.00 | 32.70 | 25.00 | 5.32 |
| IAELSATAQEIIK | Labelled | 697.90 | 968.55 | 3.50 | 10.00 | 32.70 | 25.00 | 5.32 |
| (U-13C6, 15N2) | | 697.90 | 810.48 | 3.50 | 10.00 | 32.70 | 25.00 | 5.32 |
| (SEQ ID No.: 67) | | | | | | | | |
| EQHLFLPFSYK (SEQ ID No.: 16) | Native | 470.25 | 641.33 | 3.50 | 10.00 | 19.10 | 25.00 | 5.97 |
| | | 470.25 | 544.28 | 3.50 | 10.00 | 23.10 | 25.00 | 5.97 |
| | | 470.25 | 397.21 | 3.50 | 10.00 | 27.10 | 25.00 | 5.97 |
| EQHLFLPFSYK | Labelled | 472.91 | 649.34 | 3.50 | 10.00 | 19.10 | 25.00 | 5.97 |
| (U-13C6, 15N2) | | 472.91 | 552.29 | 3.50 | 10.00 | 23.10 | 25.00 | 5.97 |
| (SEQ ID No.: 68) | | | | | | | | |
| ADQVCINLR (SEQ ID No.: 17) | Native | 544.78 | 774.43 | 3.50 | 10.00 | 29.20 | 25.00 | 4.32 |
| | | 544.78 | 675.36 | 3.50 | 10.00 | 29.20 | 25.00 | 4.32 |
| | | 544.78 | 515.33 | 3.50 | 10.00 | 29.20 | 25.00 | 4.32 |
| ADQVCINLR | Labelled | 549.78 | 784.43 | 3.50 | 10.00 | 29.20 | 25.00 | 4.32 |
| (U-13C6, 15N4) | | 549.78 | 685.36 | 3.50 | 10.00 | 29.20 | 25.00 | 4.32 |
| (SEQ ID No.: 69) | | | | | | | | |
| ILTSDVFQDCNK | Native | 720.35 | 811.34 | 3.50 | 10.00 | 34.00 | 25.00 | 4.74 |
| (SEQ ID No.: 18) | | 720.35 | 664.27 | 3.50 | 10.00 | 34.00 | 25.00 | 4.74 |
| | | 720.35 | 663.80 | 3.50 | 10.00 | 42.00 | 25.00 | 4.74 |
| ILTSDVFQDCNK | Labelled | 724.35 | 819.35 | 3.50 | 10.00 | 34.00 | 25.00 | 4.74 |
| (U-13C6, 15N2) | | 724.35 | 667.81 | 3.50 | 10.00 | 42.00 | 25.00 | 4.74 |
| (SEQ ID No.: 70) | | | | | | | | |
| YAGSQVASTSEVLK | Native | 564.77 | 696.39 | 3.50 | 10.00 | 26.20 | 25.00 | 3.89 |
| (SEQ ID No.: 19) | | 564.77 | 609.36 | 3.50 | 10.00 | 26.20 | 25.00 | 3.89 |
| | | 564.77 | 446.30 | 3.50 | 10.00 | 26.20 | 25.00 | 3.89 |
| YAGSQVASTSEVLK | Labelled | 568.78 | 704.40 | 3.50 | 10.00 | 26.20 | 25.00 | 3.89 |
| (U-13C6, 15N2) | | 568.78 | 617.37 | 3.50 | 10.00 | 26.20 | 25.00 | 3.89 |
| (SEQ ID No.: 71) | | | | | | | | |
| ESDTSYVSLK (SEQ ID No.: 20) | Native | 568.78 | 716.36 | 3.50 | 10.00 | 26.40 | 25.00 | 5.40 |
| | | 568.78 | 569.29 | 3.50 | 10.00 | 18.40 | 25.00 | 5.40 |
| | | 568.78 | 482.26 | 3.50 | 10.00 | 38.40 | 25.00 | 5.40 |
| ESDTSYVSLK | Labelled | 572.79 | 724.37 | 3.50 | 10.00 | 26.40 | 25.00 | 5.40 |
| (U-13C6, 15N2) | | 572.79 | 577.30 | 3.50 | 10.00 | 18.40 | 25.00 | 5.40 |
| (SEQ ID No.: 72) | | | | | | | | |
| GYSIFSYATK (SEQ ID No.: 21) | Native | 700.84 | 934.51 | 3.50 | 10.00 | 37.00 | 25.00 | 5.37 |
| | | 700.84 | 764.40 | 3.50 | 10.00 | 33.00 | 25.00 | 5.37 |
| | | 700.84 | 617.34 | 3.50 | 10.00 | 33.00 | 25.00 | 5.37 |
| GYSIFSYATK | Labelled | 705.84 | 944.51 | 3.50 | 10.00 | 37.00 | 25.00 | 5.37 |
| (U-13C6, 15N2) | | 705.84 | 774.41 | 3.50 | 10.00 | 33.00 | 25.00 | 5.37 |
| (SEQ ID No.: 73) | | | | | | | | |
| STDYGIFQINSR | Native | 720.37 | 933.53 | 3.50 | 10.00 | 38.00 | 25.00 | 4.11 |
| (SEQ ID No. 22) | | 720.37 | 834.46 | 3.50 | 10.00 | 38.00 | 25.00 | 4.11 |
| | | 720.37 | 763.42 | 3.50 | 10.00 | 38.00 | 25.00 | 4.11 |
| STDYGIFQINSR | Labelled | 724.37 | 941.53 | 3.50 | 10.00 | 38.00 | 25.00 | 4.11 |
| (U-13C6, 15N4) | | 724.37 | 842.47 | 3.50 | 10.00 | 38.00 | 25.00 | 4.11 |
| (SEQ ID No.: 74) | | | | | | | | |
| YWCNDGK (SEQ ID No.: 23) | Native | 471.69 | 779.31 | 3.50 | 10.00 | 21.60 | 25.00 | 2.88 |
| | | 471.69 | 593.23 | 3.50 | 10.00 | 21.60 | 25.00 | 2.88 |
| | | 471.69 | 433.20 | 3.50 | 10.00 | 25.60 | 25.00 | 2.88 |
| YWCNDGK | Labelled | 475.70 | 787.32 | 3.50 | 10.00 | 21.60 | 25.00 | 2.88 |
| (U-13C6, 15N2) | | 475.70 | 601.24 | 3.50 | 10.00 | 21.60 | 25.00 | 2.88 |
| (SEQ ID No.: 75) | | | | | | | | |
| AHVDALR (SEQ ID No.: 24) | Native | 391.22 | 573.34 | 3.50 | 10.00 | 21.60 | 25.00 | 2.39 |
| | | 391.22 | 474.27 | 3.50 | 10.00 | 25.60 | 25.00 | 2.39 |
| | | 391.22 | 359.24 | 3.50 | 10.00 | 29.60 | 25.00 | 2.39 |
| AHVDALR | Labelled | 396.22 | 583.34 | 3.50 | 10.00 | 21.60 | 25.00 | 2.39 |
| (U-13C6, 15N4) | | 396.22 | 484.27 | 3.50 | 10.00 | 25.60 | 25.00 | 2.39 |
| (SEQ ID No.: 76) | | | | | | | | |
| ATEHLSTLSEK (SEQ ID No.: 25) | Native | 405.88 | 777.44 | 3.50 | 10.00 | 24.10 | 25.00 | 3.11 |
| | | 405.88 | 664.35 | 3.50 | 10.00 | 24.10 | 25.00 | 3.11 |
| | | 405.88 | 363.19 | 3.50 | 10.00 | 24.10 | 25.00 | 3.11 |
| ATEHLSTLSEK | Labelled | 408.55 | 672.36 | 3.50 | 10.00 | 24.10 | 25.00 | 3.11 |
| (U-13C6, 15N2) | | 408.55 | 371.20 | 3.50 | 10.00 | 24.10 | 25.00 | 3.11 |
| (SEQ ID No.: 77) | | | | | | | | |
| TVVQPSVGAAAGPVVPPCPGR | Native | 672.69 | 978.52 | 3.50 | 10.00 | 24.60 | 25.00 | 4.96 |
| (SEQ ID No.: 26) | | 672.69 | 683.33 | 3.50 | 10.00 | 28.60 | 25.00 | 4.96 |
| | | 672.69 | 586.28 | 3.50 | 10.00 | 40.60 | 25.00 | 4.96 |
| TVVQPSVGAAAGPVVPPCPGR | Labelled | 676.03 | 988.52 | 3.50 | 10.00 | 24.60 | 25.00 | 4.96 |
| (U-13C6, 15N4) | | 676.03 | 693.33 | 3.50 | 10.00 | 28.60 | 25.00 | 4.96 |
| (SEQ ID No.: 78) | | | | | | | | |
| CNLLAEK (SEQ ID No.: 27) | Native | 745.85 | 1089.54 | 3.50 | 10.00 | 39.30 | 25.00 | 5.76 |
| | | 745.85 | 869.45 | 3.50 | 10.00 | 39.30 | 25.00 | 5.76 |
| | | 745.85 | 508.29 | 3.50 | 10.00 | 39.30 | 25.00 | 5.76 |
| CNLLAEK | Labelled | 750.85 | 1099.54 | 3.50 | 10.00 | 39.30 | 25.00 | 5.76 |
| (U-13C6, 15N2) | | 750.85 | 879.45 | 3.50 | 10.00 | 39.30 | 25.00 | 5.76 |
| (SEQ ID No.: 79) | | | | | | | | |
| GGEGTGYFVDFSVR | Native | 455.74 | 797.39 | 3.50 | 10.00 | 24.80 | 25.00 | 2.69 |
| (SEQ ID No.: 28) | | 455.74 | 726.35 | 3.50 | 10.00 | 24.80 | 25.00 | 2.69 |
| | | 455.74 | 611.33 | 3.50 | 10.00 | 28.80 | 25.00 | 2.69 |
| GGEGTGYFVDFSVR | Labelled | 460.74 | 807.40 | 3.50 | 10.00 | 24.80 | 25.00 | 2.69 |
| (U-13C6, 15N4) | | 460.74 | 736.36 | 3.50 | 10.00 | 24.80 | 25.00 | 2.69 |
| (SEQ ID No.: 80) | | | | | | | | |
| IADAHLDR (SEQ ID No.: 29) | Native | 424.22 | 687.40 | 3.50 | 10.00 | 23.20 | 25.00 | 3.55 |
| | | 424.22 | 573.36 | 3.50 | 10.00 | 23.20 | 25.00 | 3.55 |
| | | 424.22 | 460.28 | 3.50 | 10.00 | 23.20 | 25.00 | 3.55 |
| IADAHLDR | Labelled | 428.22 | 581.37 | 3.50 | 10.00 | 23.20 | 25.00 | 3.55 |
| (U-13C6, 15N4) | | 428.22 | 468.30 | 3.50 | 10.00 | 23.20 | 25.00 | 3.55 |
| (SEQ ID No.: 81) | | | | | | | | |
| GCPDVQASLPDAK | Native | 453.22 | 630.35 | 3.50 | 10.00 | 28.00 | 25.00 | 4.06 |
| (SEQ ID No.: 32) | | 453.22 | 430.23 | 3.50 | 10.00 | 32.00 | 25.00 | 4.06 |
| | | 453.22 | 430.14 | 3.50 | 10.00 | 44.00 | 25.00 | 4.06 |
| GCPDVQASLPDAK | Labelled | 455.89 | 638.35 | 3.50 | 10.00 | 28.00 | 25.00 | 4.06 |
| (U-13C6, 15N2) | | 455.89 | 438.24 | 3.50 | 10.00 | 32.00 | 25.00 | 4.06 |
| (SEQ ID No.: 84) | | | | | | | | |
| TFTLLDPK (SEQ ID No.: 33) | Native | 467.77 | 686.41 | 3.50 | 10.00 | 21.40 | 25.00 | 5.26 |
| | | 467.77 | 585.36 | 3.50 | 10.00 | 21.40 | 25.00 | 5.26 |
| | | 467.77 | 472.28 | 3.50 | 10.00 | 21.40 | 25.00 | 5.26 |
| TFTLLDPK | Labelled | 471.77 | 694.42 | 3.50 | 10.00 | 21.40 | 25.00 | 5.26 |
| (U-13C6, 15N2) | | 471.77 | 593.37 | 3.50 | 10.00 | 21.40 | 25.00 | 5.26 |
| (SEQ ID No.: 85) | | | | | | | | |
| GSPAINVAVHVFR | Native | 456.26 | 728.42 | 3.50 | 10.00 | 26.40 | 25.00 | 5.40 |
| (SEQ ID No.: 34) | | 456.26 | 558.31 | 3.50 | 10.00 | 18.40 | 25.00 | 5.40 |
| | | 456.26 | 611.86 | 3.50 | 10.00 | 18.40 | 25.00 | 5.40 |
| GSPAINVAVHVFR | Labelled | 459.59 | 738.42 | 3.50 | 10.00 | 26.40 | 25.00 | 5.40 |
| (U-13C6, 15N4) | | 459.59 | 616.86 | 3.50 | 10.00 | 18.40 | 25.00 | 5.40 |
| (SEQ ID No.: 86) | | | | | | | | |
| AADDTWEPFASGK | Native | 697.81 | 921.45 | 3.50 | 10.00 | 32.90 | 25.00 | 5.04 |
| (SEQ ID No.: 35) | | 697.81 | 735.37 | 3.50 | 10.00 | 32.90 | 25.00 | 5.04 |
| | | 697.81 | 606.32 | 3.50 | 10.00 | 40.90 | 25.00 | 5.04 |
| AADDTWEPFASGK | Labelled | 701.82 | 929.46 | 3.50 | 10.00 | 32.90 | 25.00 | 5.04 |
| (U-13C6, 15N2) | | 701.82 | 614.33 | 3.50 | 10.00 | 40.90 | 25.00 | 5.04 |
| (SEQ ID No.: 87) | | | | | | | | |
| DSVTGTLPK (SEQ ID No.: 36) | Native | 459.25 | 616.37 | 3.50 | 10.00 | 21.00 | 25.00 | 3.56 |
| | | 459.25 | 515.32 | 3.50 | 10.00 | 21.00 | 25.00 | 3.56 |
| | | 459.25 | 357.25 | 3.50 | 10.00 | 29.00 | 25.00 | 3.56 |
| DSVTGTLPK | Labelled | 463.25 | 624.38 | 3.50 | 10.00 | 21.00 | 25.00 | 3.56 |
| (U-13C6, 15N2) | | 463.25 | 523.33 | 3.50 | 10.00 | 21.00 | 25.00 | 3.56 |
| (SEQ ID No.: 88) | | | | | | | | |
| IAYGTQGSSGYSLR | Native | 730.36 | 1112.53 | 3.50 | 10.00 | 38.50 | 25.00 | 4.19 |
| (SEQ ID No.: 37) | | 730.36 | 954.46 | 3.50 | 10.00 | 38.50 | 25.00 | 4.19 |
| | | 730.36 | 826.41 | 3.50 | 10.00 | 42.50 | 25.00 | 4.19 |
| IAYGTQGSSGYSLR | Labelled | 735.36 | 1122.54 | 3.50 | 10.00 | 38.50 | 25.00 | 4.19 |
| (U-13C6, 15N4) | | 735.36 | 836.41 | 3.50 | 10.00 | 42.50 | 25.00 | 4.19 |
| (SEQ ID No.: 89) | | | | | | | | |
| ANRPFLVFIR (SEQ ID No.: 38) | Native | 411.58 | 647.42 | 3.50 | 10.00 | 20.30 | 25.00 | 6.00 |
| | | 411.58 | 534.34 | 3.50 | 10.00 | 20.30 | 25.00 | 6.00 |
| | | 411.58 | 435.27 | 3.50 | 10.00 | 20.30 | 25.00 | 6.00 |
| ANRPFLVFIR | Labelled | 414.91 | 544.34 | 3.50 | 10.00 | 20.30 | 25.00 | 6.00 |
| (U-13C6, 15N4) | | 414.91 | 445.27 | 3.50 | 10.00 | 20.30 | 25.00 | 6.00 |
| (SEQ ID No.: 90) | | | | | | | | |
| TSCLLFMGR (SEQ ID No.: 39) | Native | 542.77 | 736.42 | 3.50 | 10.00 | 29.10 | 25.00 | 5.48 |
| | | 542.77 | 623.33 | 3.50 | 10.00 | 29.10 | 25.00 | 5.48 |
| | | 542.77 | 510.25 | 3.50 | 10.00 | 25.10 | 25.00 | 5.48 |
| TSCLLFMGR | Labelled | 547.77 | 633.34 | 3.50 | 10.00 | 29.10 | 25.00 | 5.48 |
| (U-13C6, 15N4) | | 547.77 | 520.25 | 3.50 | 10.00 | 25.10 | 25.00 | 5.48 |
| (SEQ ID No.: 91) | | | | | | | | |
| HFQNLGK (SEQ ID No.: 40) | Native | 422.23 | 706.39 | 3.50 | 10.00 | 23.10 | 25.00 | 2.54 |
| | | 422.23 | 559.32 | 3.50 | 10.00 | 23.10 | 25.00 | 2.54 |
| | | 422.23 | 431.26 | 3.50 | 10.00 | 27.10 | 25.00 | 2.54 |
| HFQNLGK | Labelled | 426.23 | 714.40 | 3.50 | 10.00 | 23.10 | 25.00 | 2.54 |
| (U-13C6, 15N2) | | 426.23 | 567.33 | 3.50 | 10.00 | 23.10 | 25.00 | 2.54 |
| (SEQ ID No.: 92) | | | | | | | | |
| TINPAVDHCCK | Native | 438.87 | 719.26 | 3.50 | 10.00 | 25.60 | 25.00 | 2.92 |
| (SEQ ID No.: 41) | | 438.87 | 604.23 | 3.50 | 10.00 | 25.60 | 25.00 | 2.92 |
| | | 438.87 | 467.17 | 3.50 | 10.00 | 33.60 | 25.00 | 2.92 |
| TINPAVDHCCK | Labelled | 441.54 | 727.27 | 3.50 | 10.00 | 25.60 | 25.00 | 2.92 |
| (U-13C6, 15N2) | | 441.54 | 612.24 | 3.50 | 10.00 | 25.60 | 25.00 | 2.92 |
| (SEQ ID No.: 93) | | | | | | | | |
| LLDSLPSDTR (SEQ ID No.: 42) | Native | 558.80 | 890.42 | 3.50 | 10.00 | 25.90 | 25.00 | 4.38 |
| | | 558.80 | 775.39 | 3.50 | 10.00 | 33.90 | 25.00 | 4.38 |
| | | 558.80 | 575.28 | 3.50 | 10.00 | 25.90 | 25.00 | 4.38 |
| LLDSLPSDTR | Labelled | 563.80 | 900.42 | 3.50 | 10.00 | 25.90 | 25.00 | 4.38 |
| (U-13C6, 15N4) | | 563.80 | 585.28 | 3.50 | 10.00 | 25.90 | 25.00 | 4.38 |
| (SEQ ID No.: 94) | | | | | | | | |
| LVLLNAIYLSAK | Native | 659.41 | 879.49 | 3.50 | 10.00 | 31.00 | 25.00 | 6.82 |
| (SEQ ID No.: 43) | | 659.41 | 765.45 | 3.50 | 10.00 | 31.00 | 25.00 | 6.82 |
| | | 659.41 | 694.41 | 3.50 | 10.00 | 43.00 | 25.00 | 6.82 |
| LVLLNAIYLSAK | Labelled | 663.41 | 887.50 | 3.50 | 10.00 | 31.00 | 25.00 | 6.82 |
| (U-13C6, 15N2) | | 663.41 | 773.46 | 3.50 | 10.00 | 31.00 | 25.00 | 6.82 |
| (SEQ ID No.: 95) | | | | | | | | |
| ILNIFGVIK (SEQ ID No.: 44) | Native | 508.83 | 790.48 | 3.50 | 10.00 | 23.40 | 25.00 | 6.80 |
| | | 508.83 | 676.44 | 3.50 | 10.00 | 23.40 | 25.00 | 6.80 |
| | | 508.83 | 563.36 | 3.50 | 10.00 | 23.40 | 25.00 | 6.80 |
| ILNIFGVIK | Labelled | 512.83 | 798.49 | 3.50 | 10.00 | 23.40 | 25.00 | 6.80 |
| (U-13C6, 15N2) | | 512.83 | 571.36 | 3.50 | 10.00 | 23.40 | 25.00 | 6.80 |
| (SEQ ID No.: 96) | | | | | | | | |
| VSASPLLYTLIEK | Native | 717.42 | 1089.66 | 3.50 | 10.00 | 29.90 | 25.00 | 6.66 |
| (SEQ ID No.: 45) | | 717.42 | 879.52 | 3.50 | 10.00 | 37.90 | 25.00 | 6.66 |
| | | 717.42 | 766.43 | 3.50 | 10.00 | 33.90 | 25.00 | 6.66 |
| VSASPLLYTLIEK | Labelled | 721.42 | 1097.66 | 3.50 | 10.00 | 29.90 | 25.00 | 6.66 |
| (U-13C6, 15N2) | | 721.42 | 774.44 | 3.50 | 10.00 | 33.90 | 25.00 | 6.66 |
| (SEQ ID No.: 97) | | | | | | | | |
| DSGSYFCR (SEQ ID No.: 46) | Native | 496.20 | 789.33 | 3.50 | 10.00 | 22.80 | 25.00 | 3.58 |
| | | 496.20 | 645.28 | 3.50 | 10.00 | 22.80 | 25.00 | 3.58 |
| | | 496.20 | 482.22 | 3.50 | 10.00 | 22.80 | 25.00 | 3.58 |
| DSGSYFCR | Labelled | 501.20 | 799.34 | 3.50 | 10.00 | 22.80 | 25.00 | 3.58 |
| (U-13C6, 15N4) | | 501.20 | 655.28 | 3.50 | 10.00 | 22.80 | 25.00 | 3.58 |
| (SEQ ID No.: 98) | | | | | | | | |
| VLDLSCNR (SEQ D No.: 47) | Native | 488.75 | 764.34 | 3.50 | 10.00 | 22.40 | 25.00 | 3.80 |
| | | 488.75 | 649.31 | 3.50 | 10.00 | 26.40 | 25.00 | 3.80 |
| | | 488.75 | 536.22 | 3.50 | 10.00 | 22.40 | 25.00 | 3.80 |
| VLDLSCNR | Labelled | 493.75 | 774.34 | 3.50 | 10.00 | 22.40 | 25.00 | 3.80 |
| (U-13C6, 15N4) | | 493.75 | 546.23 | 3.50 | 10.00 | 22.40 | 25.00 | 3.80 |
| (SEQ ID No.: 99) | | | | | | | | |
| DFALQNPSAVPR | Native | 657.84 | 868.46 | 3.50 | 10.00 | 34.90 | 25.00 | 4.84 |
| (SEQ ID No.: 48) | | 657.84 | 740.40 | 3.50 | 10.00 | 30.90 | 25.00 | 4.84 |
| | | 657.84 | 626.36 | 3.50 | 10.00 | 34.90 | 25.00 | 4.84 |
| DFALQNPSAVPR | Labelled | 662.84 | 750.41 | 3.50 | 10.00 | 30.90 | 25.00 | 4.84 |
| (U-13C6, 15N4) | | 662.84 | 636.37 | 3.50 | 10.00 | 34.90 | 25.00 | 4.84 |
| (SEQ ID No.: 100) | | | | | | | | |
| LAELPADALGPLQR | Native | 732.41 | 1037.57 | 3.50 | 10.00 | 34.60 | 25.00 | 5.77 |
| (SEQ ID No.: 49) | | 732.41 | 754.46 | 3.50 | 10.00 | 50.60 | 25.00 | 5.77 |
| | | 732.41 | 570.34 | 3.50 | 10.00 | 34.60 | 25.00 | 5.77 |
| LAELPADALGPLQR | Labelled | 737.41 | 1047.52 | 3.50 | 10.00 | 34.60 | 25.00 | 5.77 |
| (U-13C6, 15N4) | | 737.41 | 764.46 | 3.50 | 10.00 | 50.60 | 25.00 | 5.77 |
| (SEQ ID No.: 101) | | | | | | | | |
| ASDTAMYYCAR | Native | 654.77 | 934.39 | 3.50 | 10.00 | 34.70 | 25.00 | 5.18 |
| (SEQ ID No.: 50) | | 654.77 | 863.35 | 3.50 | 10.00 | 30.70 | 25.00 | 5.18 |
| | | 654.77 | 732.31 | 3.50 | 10.00 | 30.70 | 25.00 | 5.18 |
| ASDTAMYYCAR | Labelled | 659.78 | 873.36 | 3.50 | 10.00 | 30.70 | 25.00 | 5.18 |
| (U-13C6, 15N4) | | 659.78 | 742.32 | 3.50 | 10.00 | 30.70 | 25.00 | 5.18 |
| (SEQ ID No.: 102) | | | | | | | | |
| VEGTAFVIFGIQDGEQR | Native | 622.65 | 732.33 | 3.50 | 10.00 | 26.30 | 25.00 | 6.46 |
| (SEQ ID No.: 51) | | 622.65 | 604.27 | 3.50 | 10.00 | 30.30 | 25.00 | 6.46 |
| | | 622.65 | 525.25 | 3.50 | 10.00 | 22.30 | 25.00 | 6.46 |
| VEGTAFVIFGIQDGEQR | Labelled | 625.98 | 742.33 | 3.50 | 10.00 | 26.30 | 25.00 | 6.46 |
| (U-13C6, 15N4) | | 625.98 | 530.25 | 3.50 | 10.00 | 22.30 | 25.00 | 6.46 |
| (SEQ ID No.: 103) | | | | | | | | |
| VHQYFNVELIQPGAVK | Native | 614.67 | 712.44 | 3.50 | 10.00 | 21.90 | 25.00 | 5.70 |
| (SEQ ID No.: 52) | | 614.67 | 471.29 | 3.50 | 10.00 | 33.90 | 25.00 | 5.70 |
| | | 614.67 | 509.24 | 3.50 | 10.00 | 21.90 | 25.00 | 5.70 |
| VHQYFNVELIQPGAVK | Labelled | 617.33 | 720.44 | 3.50 | 10.00 | 21.90 | 25.00 | 5.70 |
| (U-13C6, 15N2) | | 617.33 | 479.30 | 3.50 | 10.00 | 33.90 | 25.00 | 5.70 |
| (SEQ ID No.: 104) | | | | | | | | |

Table 8 shows a summary of analytical validation. The following abbreviations are used in table 8 are: LLOQ—lower limit of quantitation; ULOQ—upper limit of quantification; CV—coefficient of variation

TABLE 8

| | | | | | | | Medium | |
|---|---|---|---|---|---|---|---|---|
| | | | | | Low | | concentration | |
| | | | | | concentration | | (LLOQ + | |
| | LLOQ | ULOQ | CV | | (LLOQ) | | ULOQ)/2 | |
| | (ng/ | (ng/ | (%) | | CV | Accuracy | CV | Accuracy |
| Peptide | ml) | ml) | limit | Validation | (%) | (%) | (%) | (%) |
| AADDTWEPFASGK | 6.37 | 1630.0 | 20 | Pass | 19.2 | 90.7 | 7.2 | 98.4 |
| (SEQ ID No.: 35) | | | | | | | | |
| ADQVCINLR | 0.10 | 1630.0 | 20 | Pass | 12.5 | 96.9 | 11.2 | 97.9 |
| (SEQ ID No.: 17) | | | | | | | | |
| AHVDALR | 0.10 | 1630.0 | 20 | Pass | 2.7 | 99.4 | 2.1 | 99.2 |
| (SEQ ID No.: 24) | | | | | | | | |
| ALDFAVGEYNK | 1.59 | 1630.0 | 20 | Pass | 15.2 | 89.5 | 4.4 | 97.8 |
| (SEQ ID No.: 8) | | | | | | | | |
| ANRPFLVFIR | 0.10 | 1630.0 | 20 | Pass | 10.7 | 87.6 | 2.9 | 97.7 |
| (SEQ ID No.: 38) | | | | | | | | |
| ASDTAMYYCAR | 25.47 | 1630.0 | 20 | Pass | 18.3 | 90.4 | 11.8 | 94.7 |
| (SEQ ID No.: 50) | | | | | | | | |
| ATEHLSTLSEK | 0.40 | 1630.0 | 20 | Pass | 17.5 | 93.9 | 2.0 | 99.1 |
| (SEQ ID No.: 25) | | | | | | | | |
| CNLLAEK | 25.47 | 1630.0 | 20 | Pass | 11.3 | 99.1 | 11.3 | 97.6 |
| (SEQ ID No.: 27) | | | | | | | | |
| CQSWSSMTPHR | 25.47 | 1630.0 | 20 | Pass | 18.4 | 95.6 | 19.2 | 88.7 |
| (SEQ ID No.: 4) | | | | | | | | |
| DFALQNPSAVPR | 6.37 | 407.5 | 20 | Pass | 11.1 | 95.9 | 8.9 | 94.9 |
| (SEQ ID No.: 48) | | | | | | | | |
| DSGSYFCR | 1.59 | 25.5 | 20 | Pass | 15.7 | 96.9 | 9.8 | 97.3 |
| (SEQ ID No.: 46) | | | | | | | | |
| DSVTGTLPK | 0.10 | 407.5 | 40 | Pass | 18.3 | 93.8 | 5.7 | 97.8 |
| (SEQ ID No.: 36) | | | | | | | | |
| EAQLPVIENK | 0.02 | 1630.0 | 20 | Pass | 19.5 | 90.5 | 2.7 | 98.3 |
| (SEQ ID No.: 3) | | | | | | | | |
| EITALAPSTMK | 6.37 | 1630.0 | 40 | Pass | 3.2 | 96.7 | 2.5 | 98.5 |
| (SEQ ID No.: 5) | | | | | | | | |
| EQHLFLPFSYK | 6.37 | 407.5 | 20 | Pass | 6.8 | 93.6 | 4.0 | 98.8 |
| (SEQ ID No.: 16) | | | | | | | | |
| EQLSLLDR | 6.37 | 407.5 | 20 | Pass | 6.8 | 94.4 | 5.2 | 96.4 |
| (SEQ ID No.: 12) | | | | | | | | |
| ESDTSYVSLK | 1.59 | 407.5 | 20 | Pass | 17.2 | 94.1 | 7.7 | 98.8 |
| (SEQ ID No.: 20) | | | | | | | | |
| FNAVLTNPQGDYDTSTGK | 25.47 | 1630.0 | 20 | Pass | 28.0 | 91.1 | 15.3 | 94.0 |
| (SEQ ID No.: 6) | | | | | | | | |
| GCPDVQASLPDAK | 1.59 | 1630.0 | 40 | Pass | 25.5 | 95.8 | 19.4 | 98.7 |
| (SEQ ID No.: 32) | | | | | | | | |
| GDVAFVK | 0.40 | 1630.0 | 20 | Pass | 20.3 | 87.5 | 1.9 | 99.9 |
| (SEQ ID No.: 13) | | | | | | | | |
| GGEGTGYFVDFSVR | 6.37 | 407.5 | 20 | Pass | 17.0 | 92.0 | 12.1 | 91.1 |
| (SEQ ID No.: 28) | | | | | | | | |
| GHMLENHVER | 25.47 | 1630.0 | 20 | Pass | 13.3 | 99.3 | 1.7 | 98.8 |
| (SEQ ID No.: 2) | | | | | | | | |
| GLPNVVTSAISLPNIR | 0.40 | 1630.0 | 20 | Pass | 29.8 | 96.0 | 36.5 | 96.9 |
| (SEQ ID No.: 1) | | | | | | | | |
| GSPAINVAVHVFR | 6.37 | 1630.0 | 40 | Pass | 24.6 | 79.6 | 16.5 | 87.8 |
| (SEQ ID No.: 34) | | | | | | | | |
| GYSIFSYATK | 1.59 | 1630.0 | 20 | Pass | 8.1 | 98.4 | 3.1 | 98.7 |
| (SEQ ID No.: 21) | | | | | | | | |
| HFQNLGK | 6.37 | 1630.0 | 40 | Pass | 29.6 | 96.4 | 21.8 | 98.0 |
| (SEQ ID No.: 40) | | | | | | | | |
| IADAHLDR | 0.10 | 407.5 | 40 | Pass | 9.3 | 96.7 | 7.3 | 97.9 |
| (SEQ ID No.: 29) | | | | | | | | |
| IAELSATAQEIIK | 0.02 | 1630.0 | 40 | Pass | 12.2 | 92.8 | 7.5 | 97.0 |
| (SEQ ID No.: 15) | | | | | | | | |
| IAYGTQGSSGYSLR | 6.37 | 1630.0 | 20 | Pass | 11.6 | 97.4 | 7.4 | 94.1 |
| (SEQ ID No.: 37) | | | | | | | | |
| ILNIFGVIK | 0.02 | 407.5 | 20 | Pass | 34.2 | 97.9 | 42.1 | 99.8 |
| (SEQ ID No.: 44) | | | | | | | | |
| ILTSDVFQDCNK | not pass | | | | 85.8 | 94.5 | 25.9 | 97.0 |
| (SEQ ID No.: 18) | | | | | | | | |
| LAELPADALGPLQR | 0.10 | 1630.0 | 20 | Pass | 10.9 | 95.5 | 8.8 | 95.1 |
| (SEQ ID No.: 49) | | | | | | | | |
| LLDSLPSDTR | 0.10 | 1630.0 | 20 | Pass | 14.2 | 88.6 | 3.6 | 98.7 |
| (SEQ ID No.: 42) | | | | | | | | |
| LVGGPMDASVEEEGVRR | not pass | | | | 82.4 | 75.7 | 44.1 | 87.5 |
| (SEQ ID No.: 9) | | | | | | | | |
| LVLLNAIYLSAK | 0.40 | 1630.0 | 20 | Pass | 80.6 | 76.4 | 70.8 | 97.2 |
| (SEQ ID No.: 43) | | | | | | | | |
| SDVMYTDWK | 6.37 | 1630.0 | 20 | Pass | 18.6 | 93.9 | 4.5 | 96.5 |
| (SEQ ID No.: 10) | | | | | | | | |
| STDYGIFQINSR | 1.59 | 1630.0 | 20 | Pass | 14.3 | 85.7 | 6.5 | 98.7 |
| (SEQ ID No. 22) | | | | | | | | |
| TFTLLDPK | 6.37 | 1630.0 | 20 | Pass | 4.8 | 98.7 | 3.8 | 99.4 |
| (SEQ ID No.: 33) | | | | | | | | |
| TINPAVDHCCK | 1.59 | 1630.0 | 40 | Pass | 22.8 | 97.8 | 36.4 | 98.4 |
| (SEQ ID No.: 41) | | | | | | | | |
| TNQVNSGGVLLR | 1.59 | 1630.0 | 20 | Pass | 7.4 | 96.8 | 6.4 | 99.1 |
| (SEQ ID No.: 7) | | | | | | | | |
| TSCLLFMGR | 25.47 | 1630.0 | 20 | Pass | 15.5 | 99.3 | 10.4 | 97.8 |
| (SEQ ID No.: 39) | | | | | | | | |
| TVVQPSVGAAAGPVVPPCPGR | 6.37 | 1630.0 | 20 | Pass | 10.2 | 93.7 | 9.5 | 99.2 |
| (SEQ ID No.: 26) | | | | | | | | |
| VEGTAFVIFGIQDGEQR | not pass | | | | 20.3 | 94.5 | 17.0 | 89.0 |
| (SEQ ID No.: 51) | | | | | | | | |
| VHQYFNVELIQPGAVK | 0.81 | 13300.0 | 40 | Pass | 45.2 | 87.8 | 30.4 | 98.4 |
| (SEQ ID No.: 52) | | | | | | | | |
| VLDLSCNR | 0.02 | 1630.0 | 20 | Pass | 10.7 | 94.4 | 7.2 | 99.6 |
| (SEQ D No.: 47) | | | | | | | | |
| VSASPLLYTLIEK | 0.10 | 1630.0 | 20 | Pass | 11.1 | 96.4 | 5.9 | 97.6 |
| (SEQ ID No.: 45) | | | | | | | | |
| WCALSHHER | 6.37 | 1630.0 | 20 | Pass | 15.0 | 99.2 | 10.8 | 99.6 |
| (SEQ ID No.: 14) | | | | | | | | |
| WEMPFDPQDTHQSR | 25.47 | 1630.0 | 20 | Pass | 11.2 | 93.0 | 7.6 | 95.1 |
| (SEQ ID No.: 11) | | | | | | | | |
| YAGSQVASTSEVLK | 1.59 | 1630.0 | 20 | Pass | 16.1 | 91.2 | 5.6 | 95.8 |
| (SEQ ID No.: 19) | | | | | | | | |
| YWCNDGK (SEQ ID No.: 23) | 6.37 | 407.5 | 20 | Pass | 8.5 | 97.7 | 19.8 | 98.7 |
| Median | 1.6 | 1630.0 | — | — | 15.3 | 94.5 | 7.6 | 97.9 |
| | High | | | | | | | |
| | concentration | | Matrix effects | | | | | |
| | (ULOQ) | | | Significant | | | | |
| | CV | Accuracy | P- | matrix | Matrix | slope | slope | % |
| | (%) | (%) | value | effects? | factor | BSA | plasma | deviation |
| AADDTWEPFASGK | 10.0 | 94.5 | 0.004 | Yes | X | 0.03+/−0.006 | 0.019+/−0.001 | 157.9 |
| (SEQ ID No.: 35) | | | | | | | | |
| ADQVCINLR | 6.5 | 97.8 | 0.06 | No | — | 0.068+/−0.005 | 0.062+/−0.002 | 109.7 |
| (SEQ ID No.: 17) | | | | | | | | |
| AHVDALR | 4.5 | 99.7 | 0.98 | No | — | 0.033+/−0 | 0.031+/−0 | 106.5 |
| (SEQ ID No.: 24) | | | | | | | | |
| ALDFAVGEYNK | 5.4 | 94.4 | 0.6 | No | — | 0.024+/−0.001 | 0.024+/−0.002 | 100 |
| (SEQ ID No.: 8) | | | | | | | | |
| ANRPFLVFIR | 5.0 | 99.3 | 0.41 | No | — | 0.044+/−0.002 | 0.042+/−0.003 | 104.8 |
| (SEQ ID No.: 38) | | | | | | | | |
| ASDTAMYYCAR | 11.7 | 93.2 | 0.01 | Yes | X | 0.013+/−0.002 | 0.009+/−0 | 144.4 |
| (SEQ ID No.: 50) | | | | | | | | |
| ATEHLSTLSEK | 5.4 | 97.5 | 0.02 | Yes | X | 0.024+/−0 | 0.023+/−0.001 | 104.3 |
| (SEQ ID No.: 25) | | | | | | | | |
| CNLLAEK | 7.6 | 96.7 | 0.83 | No | — | 0.007+/−0.001 | 0.006+/−0 | 116.7 |
| (SEQ ID No.: 27) | | | | | | | | |
| CQSWSSMTPHR | 6.5 | 98.0 | 0.73 | No | — | 0.029+/−0.003 | 0.019+/−0.003 | 152.6 |
| (SEQ ID No.: 4) | | | | | | | | |
| DFALQNPSAVPR | 6.6 | 96.3 | 0.83 | No | — | 0.146+/−0.009 | 0.391+/−0.046 | 37.3 |
| (SEQ ID No.: 48) | | | | | | | | |
| DSGSYFCR | 10.6 | 98.2 | 0.45 | No | — | 0.061+/−0.003 | 0.054+/−0.004 | 113 |
| (SEQ ID No.: 46) | | | | | | | | |
| DSVTGTLPK | 5.1 | 98.7 | 0.99 | No | — | 0.114+/−0.003 | 0.133+/−0.004 | 85.7 |
| (SEQ ID No.: 36) | | | | | | | | |
| EAQLPVIENK | 1.8 | 99.8 | 0.53 | No | — | 0.022+/−0 | 0.024+/−0 | 91.7 |
| (SEQ ID No.: 3) | | | | | | | | |
| EITALAPSTMK | 17.9 | 98.6 | 0.07 | No | — | 0.027+/−0.001 | 0.035+/−0 | 77.1 |
| (SEQ ID No.: 5) | | | | | | | | |
| EQHLFLPFSYK | 4.4 | 98.4 | 0.58 | No | — | 0.02+/−0 | 0.021+/−0.001 | 95.2 |
| (SEQ ID No.: 16) | | | | | | | | |
| EQLSLLDR | 4.5 | 98.2 | 0.02 | Yes | X | 0.074+/−0.002 | 0.135+/−0.01 | 54.8 |
| (SEQ ID No.: 12) | | | | | | | | |
| ESDTSYVSLK | 4.2 | 99.1 | 0.11 | No | — | 0.116+/−0.009 | 0.198+/−0.033 | 58.6 |
| (SEQ ID No.: 20) | | | | | | | | |
| FNAVLTNPQGDYDTSTGK | 12.1 | 95.3 | 0.38 | No | — | 0.013+/−0.004 | 0.013+/−0.006 | 100 |
| (SEQ ID No.: 6) | | | | | | | | |
| GCPDVQASLPDAK | 32.3 | 97.2 | 0.72 | No | — | 0.1+/−0.006 | 0.12+/−0.01 | 83.3 |
| (SEQ ID No.: 32) | | | | | | | | |
| GDVAFVK | 6.6 | 97.8 | 0.04 | Yes | X | 0.02+/−0.001 | 0.02+/−0 | 100 |
| (SEQ ID No.: 13) | | | | | | | | |
| GGEGTGYFVDFSVR | 14.5 | 91.1 | 0.11 | No | — | 0.069+/−0.009 | 0.076+/−0.024 | 90.8 |
| (SEQ ID No.: 28) | | | | | | | | |
| GHMLENHVER | 4.9 | 99.1 | 0.72 | No | — | 0.01+/−0 | 0.01+/−0 | 100 |
| (SEQ ID No.: 2) | | | | | | | | |
| GLPNVVTSAISLPNIR | 38.5 | 99.5 | 0.45 | No | — | 0.038+/−0.002 | 0.026+/−0.001 | 146.2 |
| (SEQ ID No.: 1) | | | | | | | | |
| GSPAINVAVHVFR | 19.6 | 79.3 | 0.98 | No | — | 0.011+/−0.002 | 0.015+/−0.002 | 73.3 |
| (SEQ ID No.: 34) | | | | | | | | |
| GYSIFSYATK | 5.8 | 99.6 | 0.75 | No | — | 0.036+/−0.002 | 0.036+/−0.002 | 100 |
| (SEQ ID No.: 21) | | | | | | | | |
| HFQNLGK | 3.3 | 98.8 | 0.31 | No | — | 0.039+/−0.001 | 0.039+/−0.001 | 100 |
| (SEQ ID No.: 40) | | | | | | | | |
| IADAHLDR | 3.5 | 99.0 | 0.01 | Yes | X | 0.187+/−0.003 | 0.374+/−0.018 | 50 |
| (SEQ ID No.: 29) | | | | | | | | |
| IAELSATAQEIIK | 7.5 | 98.1 | 0.56 | No | — | 0.024+/−0.001 | 0.024+/−0.001 | 100 |
| (SEQ ID No.: 15) | | | | | | | | |
| IAYGTQGSSGYSLR | 12.7 | 85.3 | 0.27 | No | — | 0.12+/−0.027 | 0.106+/−0.017 | 113.2 |
| (SEQ ID No.: 37) | | | | | | | | |
| ILNIFGVIK | 38.7 | 97.9 | 0.23 | No | — | 0.053+/−0.001 | 0.05+/−0.001 | 106 |
| (SEQ ID No.: 44) | | | | | | | | |
| ILTSDVFQDCNK | 41.6 | 76.9 | 0.05 | No | — | 0.281+/−0.047 | 0.443+/−0.16 | 63.4 |
| (SEQ ID No.: 18) | | | | | | | | |
| LAELPADALGPLQR | 12.5 | 96.5 | 0.08 | No | — | 0.099+/−0.004 | 0.159+/−0.011 | 62.3 |
| (SEQ ID No.: 49) | | | | | | | | |
| LLDSLPSDTR | 4.1 | 98.9 | 0.19 | No | — | 0.024+/−0.001 | 0.025+/−0 | 96 |
| (SEQ ID No.: 42) | | | | | | | | |
| LVGGPMDASVEEEGVRR | 29.0 | 75.4 | | | — | | | |
| (SEQ ID No.: 9) | | | | | | | | |
| LVLLNAIYLSAK | 71.4 | 98.3 | 0.49 | No | — | 0.022+/−0.001 | 0.023+/−0.001 | 95.7 |
| (SEQ ID No.: 43) | | | | | | | | |
| SDVMYTDWK | 11.8 | 97.4 | 0.17 | No | — | 0.077+/−0.004 | 0.084+/−0.009 | 91.7 |
| (SEQ ID No.: 10) | | | | | | | | |
| STDYGIFQINSR | 5.6 | 98.4 | 0.86 | No | — | 0.011+/−0.001 | 0.011+/−0.001 | 100 |
| (SEQ ID No. 22) | | | | | | | | |
| TFTLLDPK | 5.7 | 97.1 | 0.55 | No | — | 0.01+/−0.001 | 0.009+/−0 | 111.1 |
| (SEQ ID No.: 33) | | | | | | | | |
| TINPAVDHCCK | 20.5 | 98.5 | 0.11 | No | — | 0.141+/−0.009 | 0.107+/−0.003 | 131.8 |
| (SEQ ID No.: 41) | | | | | | | | |
| TNQVNSGGVLLR | 8.8 | 98.4 | 0.23 | No | — | 0.038+/−0.002 | 0.036+/−0.003 | 105.6 |
| (SEQ ID No.: 7) | | | | | | | | |
| TSCLLFMGR | 7.5 | 99.0 | 0.69 | No | — | 0.052+/−0.003 | 0.043+/−0.001 | 120.9 |
| (SEQ ID No.: 39) | | | | | | | | |
| TVVQPSVGAAAGPVVPPCPGR | 10.1 | 96.3 | 0.93 | No | — | 0.15+/−0.014 | 0.127+/−0.011 | 118.1 |
| (SEQ ID No.: 26) | | | | | | | | |
| VEGTAFVIFGIQDGEQR | 25.4 | 98.3 | 0.58 | No | — | | | |
| (SEQ ID No.: 51) | | | | | | | | |
| VHQYFNVELIQPGAVK | 25.8 | 95.1 | 0.15 | No | — | 0.032+/−0.002 | 0.032+/−0.004 | 100 |
| (SEQ ID No.: 52) | | | | | | | | |
| VLDLSCNR | 15.8 | 99.7 | 0.51 | No | — | 0.067+/−0.001 | 0.065+/−0.001 | 103.1 |
| (SEQ D No.: 47) | | | | | | | | |
| VSASPLLYTLIEK | 9.0 | 99.1 | 0.49 | No | — | 0.083+/−0.003 | 0.081+/−0.004 | 102.5 |
| (SEQ ID No.: 45) | | | | | | | | |
| WCALSHHER | 12.6 | 97.7 | 0.4 | No | — | 0.057+/−0.002 | 0.045+/−0.001 | 126.7 |
| (SEQ ID No.: 14) | | | | | | | | |
| WEMPFDPQDTHQSR | 6.2 | 97.7 | 0.61 | No | — | 0.011+/−0.001 | 0.011+/−0.001 | 100 |
| (SEQ ID No.: 11) | | | | | | | | |
| YAGSQVASTSEVLK | 6.1 | 96.1 | 0.38 | No | — | 0.03+/−0.002 | 0.029+/−0.004 | 103.4 |
| (SEQ ID No.: 19) | | | | | | | | |
| YWCNDGK (SEQ ID No.: 23) | 10.9 | 99.0 | 0.09 | No | — | 0.439+/−0.015 | 0.569+/−0.041 | 77.2 |
| Median | 8.2 | 98.0 | — | — | — | | | |

TABLE 9
Cohort 1 - Statistical analysis results. Kendall's Tau trend test between peptide quantities and COVID19 treatment escalation score (WHO 0, 3, 4, 5, 6, 7)

| Peptide | Peptide.Genes | pval.kt | p.adjust.bh.kt | slope.kt |
|---|---|---|---|---|
| WEMPFDPQDTHQSR (SEQ ID | WEMPFDPQDTHQSR (SEQ ID No.: 11); | 5.92E−08 | 1.33E−06 | 1332.332 |
| No.: 11) | SERPINA3 | | | |
| EQLSLLDR (SEQ ID No.: 12) | EQLSLLDR (SEQ ID No.: 12); SERPINA3 | 6.63E−08 | 1.33E−06 | 264.684 |
| CNLLAEK (SEQ ID No.: 27) | CNLLAEK (SEQ ID No.: 27); AHSG | 1.80E−07 | 1.80E−06 | −1442.73 |
| GYSIFSYATK (SEQ ID No.: 21) | GYSIFSYATK (SEQ ID No.: 21); CRP | 1.80E−07 | 1.80E−06 | 208.8282 |
| TVVQPSVGAAAGPVVPPCPGR | TVVQPSVGAAAGPVVPPCPGR (SEQ ID No.: | 2.78E−07 | 2.06E−06 | −746.215 |
| (SEQ ID No.: 26) | 26); AHSG | | | |
| GDVAFVK (SEQ ID No.: 13) | GDVAFVK (SEQ ID No.: 13); TF | 3.09E−07 | 2.06E−06 | −2079.83 |
| WCALSHHER (SEQ ID No.: 14) | WCALSHHER (SEQ ID No.: 14); TF | 7.99E−07 | 4.56E−06 | −2224.65 |
| ATEHLSTLSEK (SEQ ID No.: 25) | ATEHLSTLSEK (SEQ ID No.: 25); APOA1 | 9.82E−07 | 4.91E−06 | −5266.75 |
| AHVDALR (SEQ ID No.: 24) | AHVDALR (SEQ ID No.: 24); APOA1 | 1.21E−06 | 5.36E−06 | −4172.75 |
| LVLLNAIYLSAK (SEQ ID No.: 43) | LVLLNAIYLSAK (SEQ ID No.: 43); SERPING1 | 2.97E−06 | 1.19E−05 | 689.3288 |
| ALDFAVGEYNK (SEQ ID No.: 8) | ALDFAVGEYNK (SEQ ID No.: 8); CST3 | 3.62E−06 | 1.32E−05 | 14.27221 |
| SDVMYTDWK (SEQ ID No.: 10) | SDVMYTDWK (SEQ ID No.: 10); ORM2 | 5.33E−06 | 1.78E−05 | 299.6885 |
| LLDSLPSDTR (SEQ ID No.: 42) | LLDSLPSDTR (SEQ ID No.: 42); SERPING1 | 9.42E−06 | 2.90E−05 | 167.6913 |
| YWCNDGK (SEQ ID No.: 23) | YWCNDGK (SEQ ID No.: 23); LYZ | 1.25E−05 | 3.56E−05 | 0.530168 |
| VHQYFNVELIQPGAVK (SEQ | VHQYFNVELIQPGAVK (SEQ ID No.: 52); C3 | 1.80E−05 | 4.80E−05 | 675.1894 |
| ID No.: 52) | | | | |
| GSPAINVAVHVFR (SEQ ID | GSPAINVAVHVFR (SEQ ID No.: 34); TTR | 1.21E−04 | 3.03E−04 | −251.965 |
| No.: 34) | | | | |
| GLPNVVTSAISLPNIR (SEQ ID | GLPNVVTSAISLPNIR (SEQ ID No.: 1); PRG4 | 1.55E−04 | 3.65E−04 | 8.66864 |
| No.: 1) | | | | |
| FNAVLTNPQGDYDTSTGK | FNAVLTNPQGDYDTSTGK (SEQ ID No.: 6); | 1.98E−04 | 4.16E−04 | 150.7118 |
| (SEQ ID No.: 6) | C1QC | | | |
| TFTLLDPK (SEQ ID No.: 33) | TFTLLDPK (SEQ ID No.: 33); PGLYRP2 | 1.98E−04 | 4.16E−04 | −32.7362 |
| ILTSDVFQDCNK (SEQ ID No.: 18) | ILTSDVFQDCNK (SEQ ID No.: 18); VWF | 2.14E−04 | 4.28E−04 | 0.842321 |
| LAELPADALGPLQR (SEQ ID | LAELPADALGPLQR (SEQ ID No.: 49); IGFALS | 2.51E−04 | 4.78E−04 | −58.0734 |
| No.: 49) | | | | |
| EQHLFLPFSYK (SEQ ID No.: 16) | EQHLFLPFSYK (SEQ ID No.: 16); APOB | 5.03E−04 | 9.14E−04 | 52.61636 |
| ADQVCINLR (SEQ ID No.: 17) | ADQVCINLR (SEQ ID No.: 17); EFEMP1 | 1.88E−03 | 3.27E−03 | 4.809207 |
| TNQVNSGGVLLR (SEQ ID | TNQVNSGGVLLR (SEQ ID No.: 7); C1QC | 4.34E−03 | 7.24E−03 | 31.51522 |
| No.: 7) | | | | |
| AADDTWEPFASGK (SEQ ID | AADDTWEPFASGK (SEQ ID No.: 35); TTR | 7.58E−03 | 1.21E−02 | −34.0661 |
| No.: 35) | | | | |
| GCPDVQASLPDAK (SEQ ID | GCPDVQASLPDAK (SEQ ID No.: 32); PGLYRP2 | 8.04E−03 | 0.012368 | −5.77165 |
| No.: 32) | | | | |
| ANRPFLVFIR (SEQ ID No.: 38) | ANRPFLVFIR (SEQ ID No.: 38); SERPINC1 | 0.010811 | 0.016016 | −125.755 |
| GHMLENHVER (SEQ ID No.: 2) | GHMLENHVER (SEQ ID No.: 2); ITIH1 | 0.011454 | 0.016363 | −107.945 |
| TINPAVDHCCK (SEQ ID No.: 41) | TINPAVDHCCK (SEQ ID No.: 41); AFM | 0.014381 | 0.019835 | −74.1523 |
| HFQNLGK (SEQ ID No.: 40) | HFQNLGK (SEQ ID No.: 40); AFM | 0.01699 | 0.022653 | −16.8833 |
| IAYGTQGSSGYSLR (SEQ ID | IAYGTQGSSGYSLR (SEQ ID No.: 37); KLKB1 | 0.017948 | 0.023158 | −16.5593 |
| No.: 37) | | | | |
| ESDTSYVSLK (SEQ ID No.: 20) | ESDTSYVSLK (SEQ ID No.: 20); CRP | 0.02111 | 0.026387 | 87.19703 |
| IAELSATAQEIIK (SEQ ID No.: 15) | IAELSATAQEIIK (SEQ ID No.: 15); APOB | 0.065656 | 0.079583 | 65.92091 |
| STDYGIFQINSR (SEQ ID No.: 22) | STDYGIFQINSR (SEQ ID No.: 22); LYZ | 0.292861 | 0.344543 | −12.2537 |
| TSCLLFMGR (SEQ ID No.: 39) | TSCLLFMGR (SEQ ID No.: 39); SERPIND1 | 0.352114 | 0.402416 | 5.528197 |
| ILNIFGVIK (SEQ ID No.: 44) | ILNIFGVIK (SEQ ID No.: 44); TFRC | 0.504434 | 0.560482 | −0.33407 |
| DFALQNPSAVPR (SEQ ID No.: 48) | DFALQNPSAVPR (SEQ ID No.: 48); IGFALS | 0.734001 | 0.785476 | 3.771921 |
| EAQLPVIENK (SEQ ID No.: 3) | EAQLPVIENK (SEQ ID No.: 3); PLG | 0.746202 | 0.785476 | −14.3991 |
| GGEGTGYFVDFSVR (SEQ ID | GGEGTGYFVDFSVR (SEQ ID No.: 28); HRG | 0.823917 | 0.845043 | −19.6409 |
| No.: 28) | | | | |
| CQSWSSMTPHR (SEQ ID No.: 4) | CQSWSSMTPHR (SEQ ID No.: 4); PLG | 0.871443 | 0.871443 | −6.77322 |

Cohort 2 - Statistical analysis results. Kendall's Tau trend test between peptide quantities and COVID19 treatment escalation score (WHO 3, 4, 5, 6, 7)

| | | | adjusted | |
|---|---|---|---|---|
| Peptide | Peptide.Genes | p.palue | p.value | slope.kt |
| TFTLLDPK (SEQ ID No.: 33) | TFTLLDPK (SEQ ID No.: 33); PGLYRP2 | 1.73E−16 | 8.29E−15 | −232.59 |
| GCPDVQASLPDAK (SEQ ID | GCPDVQASLPDAK (SEQ ID No.: 32); | 5.21E−16 | 1.25E−14 | −171.366 |
| No.: 32) | PGLYRP2 | | | |
| ESDTSYVSLK (SEQ ID No.: 20) | ESDTSYVSLK (SEQ ID No.: 20); CRP | 2.22E−14 | 2.66E−13 | 2388.399 |
| GYSIFSYATK (SEQ ID No.: 21) | GYSIFSYATK (SEQ ID No.: 21); CRP | 2.22E−14 | 2.66E−13 | 317.1732 |
| CNLLAEK (SEQ ID No.: 27) | CNLLAEK (SEQ ID No.: 27); AHSG | 5.18E−13 | 4.97E−12 | −20167.6 |
| TVVQPSVGAAAGPVVPPCPGR | TVVQPSVGAAAGPVVPPCPGR (SEQ ID No.: | 1.43E−12 | 1.14E−11 | −1445.22 |
| (SEQ ID No.: 26) | 26); AHSG | | | |
| GDVAFVK (SEQ ID No.: 13) | GDVAFVK (SEQ ID No.: 13); TF | 3.70E−11 | 2.54E−10 | −3235.65 |
| DSVTGTLPK (SEQ ID No.: 36) | DSVTGTLPK (SEQ ID No.: 36); KLKB1 | 5.47E−11 | 3.28E−10 | −59.9985 |
| AHVDALR (SEQ ID No.: 24) | AHVDALR (SEQ ID No.: 24); APOA1 | 7.50E−10 | 3.73E−09 | −6118.33 |
| ATEHLSTLSEK (SEQ ID No.: 25) | ATEHLSTLSEK (SEQ ID No.: 25); APOA1 | 7.78E−10 | 3.73E−09 | −8823.07 |
| WCALSHHER (SEQ ID No.: 14) | WCALSHHER (SEQ ID No.: 14); TF | 1.63E−09 | 7.13E−09 | −5238.31 |
| LAELPADALGPLQR (SEQ ID | LAELPADALGPLQR (SEQ ID No.: 49); | 4.32E−09 | 1.73E−08 | −55.1319 |
| No.: 49) | IGFALS | | | |
| DFALQNPSAVPR (SEQ ID No.: 48) | DFALQNPSAVPR (SEQ ID No.: 48); IGFALS | 6.33E−09 | 2.34E−08 | −29.6012 |
| TINPAVDHCCK (SEQ ID No.: 41) | TINPAVDHCCK (SEQ ID No.: 41); AFM | 5.50E−08 | 1.89E−07 | −550.3 |
| EQLSLLDR (SEQ ID No.: 12) | EQLSLLDR (SEQ ID No.: 12); SERPINA3 | 1.31E−07 | 4.18E−07 | 2625.823 |
| HFQNLGK (SEQ ID No.: 40) | HFQNLGK (SEQ ID No.: 40); AFM | 2.37E−07 | 6.90E−07 | −54.3341 |
| GHMLENHVER (SEQ ID No.: 2) | GHMLENHVER (SEQ ID No.: 2); ITIH1 | 2.44E−07 | 6.90E−07 | −517.84 |
| WEMPFDPQDTHQSR (SEQ ID | WEMPFDPQDTHQSR (SEQ ID No.: 11); | 7.50E−07 | 2.00E−06 | 1039.275 |
| No.: 11) | SERPINA3 | | | |
| EITALAPSTMK (SEQ ID No.: 5) | EITALAPSTMK (SEQ ID No.: 5); ACTA1; | 9.37E−07 | 2.37E−06 | 23.97025 |
| | ACTA2; ACTB; ACTC1; ACTG1; ACTG2 | | | |
| ALDFAVGEYNK (SEQ ID No.: 8) | ALDFAVGEYNK (SEQ ID No.: 8); CST3 | 1.45E−06 | 3.49E−06 | 31.13371 |
| VLDLSCNR (SEQ ID No.: 47) | VLDLSCNR (SEQ ID No.: 47); CD14 | 2.37E−06 | 5.41E−06 | 13.53262 |
| ANRPFLVFIR (SEQ ID No.: 38) | ANRPFLVFIR (SEQ ID No.: 38); SERPINC1 | 4.28E−06 | 8.92E−06 | −314.623 |
| IADAHLDR (SEQ ID No.: 29) | IADAHLDR (SEQ ID No.: 29); HRG | 4.28E−06 | 8.92E−06 | −51.6363 |
| IAYGTQGSSGYSLR (SEQ ID | IAYGTQGSSGYSLR (SEQ ID No.: 37); KLKB1 | 1.20E−05 | 2.40E−05 | −37.7417 |
| No.: 37) | | | | |
| VEGTAFVIFGIQDGEQR (SEQ | VEGTAFVIFGIQDGEQR (SEQ ID No.: 51); C3 | 1.85E−05 | 3.56E−05 | 798.2789 |
| ID No.: 51) | | | | |
| YAGSQVASTSEVLK (SEQ ID | YAGSQVASTSEVLK (SEQ ID No.: 19); VWF | 2.06E−05 | 3.79E−05 | 25.56539 |
| No.: 19) | | | | |
| TSCLLFMGR (SEQ ID No.: 39) | TSCLLFMGR (SEQ ID No.: 39); SERPIND1 | 6.30E−05 | 0.000112 | −84.4991 |
| EAQLPVIENK (SEQ ID No.: 3) | EAQLPVIENK (SEQ ID No.: 3); PLG | 0.000861 | 0.001476 | −222.069 |
| GGEGTGYFVDFSVR (SEQ ID | GGEGTGYFVDFSVR (SEQ ID No.: 28); HRG | 0.002483 | 0.004109 | −171.783 |
| No.: 28) | | | | |
| IAELSATAQEIIK (SEQ ID No.: 15) | IAELSATAQEIIK (SEQ ID No.: 15); APOB | 0.005025 | 0.00804 | −80.0036 |
| ADQVCINLR (SEQ ID No.: 17) | ADQVCINLR (SEQ ID No.: 17); EFEMP1 | 0.005745 | 0.008895 | 7.558618 |
| AADDTWEPFASGK (SEQ ID | AADDTWEPFASGK (SEQ ID No.: 35); TTR | 0.008423 | 0.012635 | −27.1604 |
| No.: 35) | | | | |
| ILTSDVFQDCNK (SEQ ID No.: 18) | ILTSDVFQDCNK (SEQ ID No.: 18); VWF | 0.017321 | 0.025194 | 10.14308 |
| GSPAINVAVHVFR (SEQ ID | GSPAINVAVHVFR (SEQ ID No.: 34); TTR | 0.039824 | 0.056222 | −104.475 |
| No.: 34) | | | | |
| EQHLFLPFSYK (SEQ ID No.: 16) | EQHLFLPFSYK (SEQ ID No.: 16); APOB | 0.055657 | 0.076206 | 286.7285 |
| CQSWSSMTPHR (SEQ ID No.: 4) | CQSWSSMTPHR (SEQ ID No.: 4); PLG | 0.057154 | 0.076206 | −713.482 |
| LLDSLPSDTR (SEQ ID No.: 42) | LLDSLPSDTR (SEQ ID No.: 42); SERPING1 | 0.059463 | 0.077108 | 116.5034 |
| YWCNDGK (SEQ ID No.: 23) | YWCNDGK (SEQ ID No.: 23); LYZ | 0.061044 | 0.077108 | 6.794503 |
| LVLLNAIYLSAK (SEQ ID No.: 43) | LVLLNAIYLSAK (SEQ ID No.: 43); SERPING1 | 0.253996 | 0.31261 | 448.8475 |
| FNAVLTNPQGDYDTSTGK | FNAVLTNPQGDYDTSTGK (SEQ ID No.: 6); | 0.280187 | 0.336224 | −124.192 |
| (SEQ ID No.: 6) | C1QC | | | |
| STDYGIFQINSR (SEQ ID No.: 22) | STDYGIFQINSR (SEQ ID No.: 22); LYZ | 0.301334 | 0.352781 | 19.95125 |
| VHQYFNVELIQPGAVK (SEQ | VHQYFNVELIQPGAVK (SEQ ID No.: 52); C3 | 0.425932 | 0.486779 | −341.517 |
| ID No.: 52) | | | | |
| VSASPLLYTLIEK (SEQ ID No.: 45) | VSASPLLYTLIEK (SEQ ID No.: 45); TFRC | 0.608057 | 0.678762 | 0.295307 |
| ILNIFGVIK (SEQ ID No.: 44) | ILNIFGVIK (SEQ ID No.: 44); TFRC | 0.736992 | 0.797797 | −0.16742 |
| SDVMYTDWK (SEQ ID No.: 10) | SDVMYTDWK (SEQ ID No.: 10); ORM2 | 0.747934 | 0.797797 | 86.59714 |
| GLPNVVTSAISLPNIR (SEQ ID | GLPNVVTSAISLPNIR (SEQ ID No.: 1); PRG4 | 0.848462 | 0.885351 | 0.976178 |
| No.: 1) | | | | |
| TNQVNSGGVLLR (SEQ ID | TNQVNSGGVLLR (SEQ ID No.: 7); C1QC | 0.891763 | 0.907806 | −4.61482 |
| No.: 7) | | | | |
| DSGSYFCR (SEQ ID No.: 46) | DSGSYFCR (SEQ ID No.: 46); FCGR3A | 0.907806 | 0.907806 | −0.1716 |

1. A method for predicting and/or classifying the severity of COVID-19 disease in a subject, the method comprising:
(i) preparing a biological sample from the subject for assay by incubating the sample with a protease to form a proteolytic digest of proteins in the sample; and
(ii) assaying the proteolytic digest of proteins of step (i) for the presence of a proteolytic peptide of at least one protein selected from the group of proteins as shown in Table 1, said group consisting of Proteoglycan 4, Inter-alpha-trypsin inhibitor heavy chain H1, Plasminogen (EC 3.4.21.7), Actin (Actin, aortic smooth muscle; Actin, cytoplasmic 1; Actin, cytoplasmic 2; Actin, gamma-enteric smooth muscle), Complement C1q subcomponent subunit C, Cystatin-C, Protein ORM2, Alpha-1-antichymotrypsin, Serotransferrin, Apolipoprotein B-100, EGF-containing fibulin-like extracellular matrix protein 1, von Willebrand factor, C-reactive protein, Lysozyme C (EC 3.2.1.17), Apolipoprotein A-I, Alpha-2-HS-glycoprotein, Histidine-rich glycoprotein, Beta-2-microglobulin, N-acetylmuramoyl-L-alanine amidase (EC 3.5.1.28), Transthyretin, Plasma kallikrein (EC 3.4.21.34), Antithrombin-III, Heparin cofactor 2, Afamin, Plasma protease C1 inhibitor, Transferrin receptor protein 1, Low affinity immunoglobulin gamma Fc region receptor III-A, Monocyte differentiation antigen CD14, Insulin-like growth factor-binding protein complex acid labile subunit, Immunoglobulin heavy variable 5-51, or Complement C3,
wherein the presence of said proteolytic peptide is assayed for using mass spectrometry with reference to a corresponding labelled and/or unlabelled reference proteolytic peptide.

2. The method of claim 1 comprising:
(ii) assaying the proteolytic digest of proteins of step (i) for the presence of one or more proteolytic peptides as shown in Table 9 Cohort 1, said group consisting of:
(SEQ ID No.: 11)
WEMPFDPQDTHQSR
(SEQ ID No.: 12)
EQLSLLDR
(SEQ ID No.: 27)
CNLLAEK
(SEQ ID No.: 21)
GYSIFSYATK
(SEQ ID No.: 26)
TVVQPSVGAAAGPVVPPCPGR
(SEQ ID No.: 13)
GDVAFVK
(SEQ ID No.: 14)
WCALSHHER
(SEQ ID No.: 25)
ATEHLSTLSEK
(SEQ ID No.: 24)
AHVDALR
(SEQ ID No.: 43)
LVLLNAIYLSAK
(SEQ ID No.: 8)
ALDFAVGEYNK
(SEQ ID No.: 10)
SDVMYTDWK
(SEQ ID No.: 42)
LLDSLPSDTR
(SEQ ID No.: 23)
YWCNDGK
(SEQ ID No.: 52)
VHQYFNVELIQPGAVK
(SEQ ID No.: 34)
GSPAINVAVHVFR
(SEQ ID No.: 1)
GLPNWTSAISLPNIR
(SEQ ID No.: 6)
FNAVLTNPQGDYDTSTGK
(SEQ ID No.: 33)
TFTLLDPK
(SEQ ID No.: 18)
ILTSDVFQDCNK
(SEQ ID No.: 49)
LAELPADALGPLQR
(SEQ ID No.: 16)
EQHLFLPFSYK
(SEQ ID No.: 17)
ADQVCINLR
(SEQ ID No.: 7)
TNQVNSGGVLLR
(SEQ ID No.: 35)
AADDTWEPFASGK
(SEQ ID No.: 32)
GCPDVQASLPDAK
(SEQ ID No.: 38)
ANRPFLVFIR
(SEQ ID No.: 2)
GHMLENHVER
(SEQ ID No.: 41)
TINPAVDHCCK
(SEQ ID No.: 40)
HFQNLGK
(SEQ ID No.: 37)
IAYGTQGSSGYSLR
(SEQ ID No.: 20)
ESDTSYVSLK
(SEQ ID No.: 15)
IAELSATAQEIIK
(SEQ ID No.: 22)
STDYGIFQINSR
(SEQ ID No.: 39)
TSCLLFMGR
(SEQ ID No.: 44)
ILNIFGVIK
(SEQ ID No.: 48)
DFALQNPSAVPR
(SEQ ID No.: 3)
EAQLPVIENK
(SEQ ID No.: 28)
GGEGTGYFVDFSVR
and
(SEQ ID No.: 4)
CQSWSSMTPHR.

3. The method of claim 1 comprising:
(ii) assaying the proteolytic digest of proteins of step (i) for the presence of one or more proteolytic peptides as shown in Table 9 Cohort 2, said group consisting of:
(SEQ ID No.: 33)
TFTLLDPK
(SEQ ID No.: 32)
GCPDVQASLPDAK
(SEQ ID No.: 20)
ESDTSYVSLK
(SEQ ID No.: 21)
GYSIFSYATK
(SEQ ID No.: 27)
CNLLAEK
(SEQ ID No.: 26)
TVVQPSVGAAAGPVVPPCPGR
(SEQ ID No.: 13)
GDVAFVK
(SEQ ID No.: 36)
DSVTGTLPK
(SEQ ID No.: 24)
AHVDALR
(SEQ ID No.: 25)
ATEHLSTLSEK
(SEQ ID No.: 14)
WCALSHHER
(SEQ ID No.: 49)
LAELPADALGPLQR
(SEQ ID No.: 48)
DFALQNPSAVPR
(SEQ ID No.: 41)
TINPAVDHCCK
(SEQ ID No.: 12)
EQLSLLDR
(SEQ ID No.: 40)
HFQNLGK
(SEQ ID No.: 2)
GHMLENHVER
(SEQ ID No.: 11)
WEMPFDPQDTHQSR
(SEQ ID No.: 5)
EITALAPSTMK
(SEQ ID No.: 8)
ALDFAVGEYNK
(SEQ ID No.: 47)
VLDLSCNR
(SEQ ID No.: 38)
ANRPFLVFIR
(SEQ ID No.: 29)
IADAHLDR
(SEQ ID No.: 37)
IAYGTQGSSGYSLR
(SEQ ID No.: 51)
VEGTAFVIFGIQDGEQR
(SEQ ID No.: 19)
YAGSQVASTSEVLK
(SEQ ID No.: 39)
TSCLLFMGR
(SEQ ID No.: 3)
EAQLPVIENK
(SEQ ID No.: 28)
GGEGTGYFVDFSVR
(SEQ ID No.: 15)
IAELSATAQEIIK
(SEQ ID No.: 17)
ADQVCINLR
(SEQ ID No.: 35)
AADDTWEPFASGK
(SEQ ID No.: 18)
ILTSDVFQDCNK
(SEQ ID No.: 34)
GSPAINVAVHVFR
(SEQ ID No.: 16)
EQHLFLPFSYK
(SEQ ID No.: 4)
CQSWSSMTPHR
(SEQ ID No.: 42)
LLDSLPSDTR
(SEQ ID No.: 23)
YWCNDGK
(SEQ ID No.: 43)
LVLLNAIYLSAK
(SEQ ID No.: 6)
FNAVLTNPQGDYDTSTGK
(SEQ ID No.: 22)
STDYGIFQINSR
(SEQ ID No.: 52)
VHQYFNVELIQPGAVK
(SEQ ID No.: 45)
VSASPLLYTLIEK
(SEQ ID No.: 44)
ILNIFGVIK
(SEQ ID No.: 10)
SDVMYTDWK
(SEQ ID No.: 1)
GLPNWTSAISLPNIR
(SEQ ID No.: 7)
TNQVNSGGVLLR
and
(SEQ ID No.: 46)
DSGSYFCR.

5. The method of claim 1 comprising:
(ii) assaying the proteolytic digest of proteins of step (i) for the presence of one or more proteolytic peptides as shown in the top-right panel of Supplementary FIG. 4, said group consisting of:
(SEQ ID No.: 32)
GCPDVQASLPDAK
(SEQ ID No.: 20)
ESDTSYVSLK
(SEQ ID No.: 27)
CNLLAEK
(SEQ ID No.: 26)
TWQPSVGAAAGPWPPCPGR
(SEQ ID No.: 4)
CQSWSSMTPHR
(SEQ ID No.: 8)
ALDFAVGEYNK
(SEQ ID No.: 13)
GDVAFVK
(SEQ ID No.: 38)
ANRPFLVFIR
(SEQ ID No.: 21)
GYSIFSYATK
(SEQ ID No.: 3)
EAQLPVIENK
(SEQ ID No.: 14)
WCALSHHER
(SEQ ID No.: 24)
AHVDALR
(SEQ ID No.: 49)
LAELPADALGPLQR
(SEQ ID No.: 29)
IADAHLDR
(SEQ ID No.: 48)
DFALQNPSAVPR.

6. A method for the treatment of a subject with COVID-19 disease, the method comprising:
(i) preparing a biological sample from the subject for assay by incubating the sample with a protease to form a proteolytic digest of proteins in the sample;
(ii) assaying the proteolytic digest of proteins of step (i) for the presence of a proteolytic peptide of at least one protein selected from the group of proteins as shown in Table 1, said group consisting of Proteoglycan 4, Inter-alpha-trypsin inhibitor heavy chain H1, Plasminogen (EC 3.4.21.7), Actin (Actin, aortic smooth muscle; Actin, cytoplasmic 1; Actin, cytoplasmic 2; Actin, gamma-enteric smooth muscle), Complement C1q subcomponent subunit C, Cystatin-C, Protein ORM2, Alpha-1-antichymotrypsin, Serotransferrin, Apolipoprotein B-100, EGF-containing fibulin-like extracellular matrix protein 1, von Willebrand factor, C-reactive protein, Lysozyme C (EC 3.2.1.17), Apolipoprotein A-I, Alpha-2-HS-glycoprotein, Histidine-rich glycoprotein, Beta-2-microglobulin, N-acetylmuramoyl-L-alanine amidase (EC 3.5.1.28), Transthyretin, Plasma kallikrein (EC 3.4.21.34), Antithrombin-III, Heparin cofactor 2, Afamin, Plasma protease C1 inhibitor, Transferrin receptor protein 1, Low affinity immunoglobulin gamma Fc region receptor III-A, Monocyte differentiation antigen CD14, Insulin-like growth factor-binding protein complex acid labile subunit, Immunoglobulin heavy variable 5-51, or Complement C3,
wherein the presence of said proteolytic peptide is assayed for using mass spectrometry with reference to a corresponding labelled and/or unlabelled reference proteolytic peptide; and
(iii) treating the subject with a therapeutic agent or treatment according to the severity of the COVID-19 disease detected in the subject.

7. A kit for predicting and/or classifying the severity of COVID-19 disease in a subject according to a method as claimed in claim 1, comprising:
a plurality of sample preparation media for analysis of a sample by mass spectrometry for the presence of a proteolytic peptide of at least one protein selected from the group of proteins as shown in Table 1, said group consisting of Proteoglycan 4, Inter-alpha-trypsin inhibitor heavy chain H1, Plasminogen (EC 3.4.21.7), Actin (Actin, aortic smooth muscle; Actin, cytoplasmic 1; Actin, cytoplasmic 2; Actin, gamma-enteric smooth muscle), Complement C1q subcomponent subunit C, Cystatin-C, Protein ORM2, Alpha-1-antichymotrypsin, Serotransferrin, Apolipoprotein B-100, EGF-containing fibulin-like extracellular matrix protein 1, von Willebrand factor, C-reactive protein, Lysozyme C (EC 3.2.1.17), Apolipoprotein A-I, Alpha-2-HS-glycoprotein, Histidine-rich glycoprotein, Beta-2-microglobulin, N-acetylmuramoyl-L-alanine amidase (EC 3.5.1.28), Transthyretin, Plasma kallikrein (EC 3.4.21.34), Antithrombin-III, Heparin cofactor 2, Afamin, Plasma protease C1 inhibitor, Transferrin receptor protein 1, Low affinity immunoglobulin gamma Fc region receptor III-A, Monocyte differentiation antigen CD14, Insulin-like growth factor-binding protein complex acid labile subunit, Immunoglobulin heavy variable 5-51, or Complement C3.

8. A pharmaceutical composition comprising a therapeutic agent for use in a method of treatment of a subject with COVID-19 disease, wherein the COVID-19 disease of the subject has been classified according to a method as claimed in claim 1.

9. A kit for use in the treatment of a subject with COVID-19 disease according to a method as claimed in claim 6, comprising:
(i) a plurality of sample preparation media for analysis of a sample by mass spectrometry for the presence of a proteolytic peptide of at least one protein selected from the group of proteins as shown in Table 1, said group consisting of Proteoglycan 4, Inter-alpha-trypsin inhibitor heavy chain H1, Plasminogen (EC 3.4.21.7), Actin (Actin, aortic smooth muscle; Actin, cytoplasmic 1; Actin, cytoplasmic 2; Actin, gamma-enteric smooth muscle), Complement C1q subcomponent subunit C, Cystatin-C, Protein ORM2, Alpha-1-antichymotrypsin, Serotransferrin, Apolipoprotein B-100, EGF-containing fibulin-like extracellular matrix protein 1, von Willebrand factor, C-reactive protein, Lysozyme C (EC 3.2.1.17), Apolipoprotein A-I, Alpha-2-HS-glycoprotein, Histidine-rich glycoprotein, Beta-2-microglobulin, N-acetylmuramoyl-L-alanine amidase (EC 3.5.1.28), Transthyretin, Plasma kallikrein (EC 3.4.21.34), Antithrombin-III, Heparin cofactor 2, Afamin, Plasma protease C1 inhibitor, Transferrin receptor protein 1, Low affinity immunoglobulin gamma Fc region receptor III-A, Monocyte differentiation antigen CD14, Insulin-like growth factor-binding protein complex acid labile subunit, Immunoglobulin heavy variable 5-51, or Complement C3; and
(ii) a therapeutic agent for treatment of COVID-19 disease.


SEQUENCE LISTING (104 sequences)

SEQ ID NO: 1 - protein, 16 aa, Homo sapiens
GLPNVVTSAISLPNIR

SEQ ID NO: 2 - protein, 10 aa, Homo sapiens
GHMLENHVER

SEQ ID NO: 3 - protein, 10 aa, Homo sapiens
EAQLPVIENK

SEQ ID NO: 4 - protein, 11 aa, Homo sapiens
CQSWSSMTPHR

SEQ ID NO: 5 - protein, 11 aa, Homo sapiens
EITALAPSTMK

SEQ ID NO: 6 - protein, 18 aa, Homo sapiens
FNAVLTNPQGDYDTSTGK

SEQ ID NO: 7 - protein, 12 aa, Homo sapiens
TNQVNSGGVLLR

SEQ ID NO: 8 - protein, 11 aa, Homo sapiens
ALDFAVGEYNK

SEQ ID NO: 9 - protein, 17 aa, Homo sapiens
LVGGPMDASVEEEGVRR

SEQ ID NO: 10 - protein, 9 aa, Homo sapiens
SDVMYTDWK

SEQ ID NO: 11 - protein, 14 aa, Homo sapiens
WEMPFDPQDTHQSR

SEQ ID NO: 12 - protein, 8 aa, Homo sapiens
EQLSLLDR

SEQ ID NO: 13 - protein, 7 aa, Homo sapiens
GDVAFVK

SEQ ID NO: 14 - protein, 9 aa, Homo sapiens
WCALSHHER

SEQ ID NO: 15 - protein, 13 aa, Homo sapiens
IAELSATAQEIIK

SEQ ID NO: 16 - protein, 11 aa, Homo sapiens
EQHLFLPFSYK

SEQ ID NO: 17 - protein, 9 aa, Homo sapiens
ADQVCINLR

SEQ ID NO: 18 - protein, 12 aa, Homo sapiens
ILTSDVFQDCNK

SEQ ID NO: 19 - protein, 14 aa, Homo sapiens
YAGSQVASTSEVLK

SEQ ID NO: 20 - protein, 10 aa, Homo sapiens
ESDTSYVSLK

SEQ ID NO: 21 - protein, 10 aa, Homo sapiens
GYSIFSYATK

SEQ ID NO: 22 - protein, 12 aa, Homo sapiens
STDYGIFQINSR

SEQ ID NO: 23 - protein, 7 aa, Homo sapiens
YWCNDGK

SEQ ID NO: 24 - protein, 7 aa, Homo sapiens
AHVDALR

SEQ ID NO: 25 - protein, 11 aa, Homo sapiens
ATEHLSTLSEK

SEQ ID NO: 26 - protein, 21 aa, Homo sapiens
TVVQPSVGAAAGPVVPPCPGR

SEQ ID NO: 27 - protein, 7 aa, Homo sapiens
CNLLAEK

SEQ ID NO: 28 - protein, 14 aa, Homo sapiens
GGEGTGYFVDFSVR

SEQ ID NO: 29 - protein, 8 aa, Homo sapiens
IADAHLDR

SEQ ID NO: 30 - protein, 10 aa, Homo sapiens
VEHSDLSFSK

SEQ ID NO: 31 - protein, 10 aa, Homo sapiens
VNHVTLSQPK

SEQ ID NO: 32 - protein, 13 aa, Homo sapiens
GCPDVQASLPDAK

SEQ ID NO: 33 - protein, 8 aa, Homo sapiens
TFTLLDPK

SEQ ID NO: 34 - protein, 13 aa, Homo sapiens
GSPAINVAVHVFR

SEQ ID NO: 35 - protein, 13 aa, Homo sapiens
AADDTWEPFASGK

SEQ ID NO: 36 - protein, 9 aa, Homo sapiens
DSVTGTLPK

SEQ ID NO: 37 - protein, 14 aa, Homo sapiens
IAYGTQGSSGYSLR

SEQ ID NO: 38 - protein, 10 aa, Homo sapiens
ANRPFLVFIR

SEQ ID NO: 39 - protein, 9 aa, Homo sapiens
TSCLLFMGR

SEQ ID NO: 40 - protein, 7 aa, Homo sapiens
HFQNLGK

SEQ ID NO: 41 - protein, 11 aa, Homo sapiens
TINPAVDHCCK

SEQ ID NO: 42 - protein, 10 aa, Homo sapiens
LLDSLPSDTR

SEQ ID NO: 43 - protein, 12 aa, Homo sapiens
LVLLNAIYLSAK

SEQ ID NO: 44 - protein, 9 aa, Homo sapiens
ILNIFGVIK

SEQ ID NO: 45 - protein, 13 aa, Homo sapiens
VSASPLLYTLIEK

SEQ ID NO: 46 - protein, 8 aa, Homo sapiens
DSGSYFCR

SEQ ID NO: 47 - protein, 8 aa, Homo sapiens
VLDLSCNR

SEQ ID NO: 48 - protein, 12 aa, Homo sapiens
DFALQNPSAVPR

SEQ ID NO: 49 - protein, 14 aa, Homo sapiens
LAELPADALGPLQR

SEQ ID NO: 50 - protein, 11 aa, Homo sapiens
ASDTAMYYCAR

SEQ ID NO: 51 - protein, 17 aa, Homo sapiens
VEGTAFVIFGIQDGEQR

SEQ ID NO: 52 - protein, 16 aa, Homo sapiens
VHQYFNVELIQPGAVK

SEQ ID NO: 53 - protein, 21 aa, Artificial sequence
  heavy isotope-labelled peptide internal standard sequence
  MISC_FEATURE (19)..(22): U-13C6,15N4
SILWRGLPNVVTSAISLPNIR

SEQ ID NO: 54 - protein, 15 aa, Artificial sequence
  heavy isotope-labelled peptide internal standard sequence
  MISC_FEATURE (9)..(11): U-13C6,15N4
GHMLENHVERLWAYL

SEQ ID NO: 55 - protein, 15 aa, Artificial sequence
  heavy isotope-labelled peptide internal standard sequence
  MISC_FEATURE (14)..(16): U-13C6,15N2
AGLLKEAQLPVIENK

SEQ ID NO: 56 - protein, 14 aa, Artificial sequence
  heavy isotope-labelled peptide internal standard sequence
  MISC_FEATURE (10)..(12): U-13C6,15N4
CQSWSSMTPHRHQK

SEQ ID NO: 57 - protein, 14 aa, Artificial sequence
  heavy isotope-labelled peptide internal standard sequence
  MISC_FEATURE (13)..(15): U-13C6,15N2
MQKEITALAPSTMK

SEQ ID NO: 58 - protein, 23 aa, Artificial sequence
  heavy isotope-labelled peptide internal standard sequence
  MISC_FEATURE (22)..(24): U-13C6,15N2
NSLIRFNAVLTNPQGDYDTSTGK

SEQ ID NO: 59 - protein, 17 aa, Artificial sequence
  heavy isotope-labelled peptide internal standard sequence
  MISC_FEATURE (11)..(13): U-13C6,15N4
TNQVNSGGVLLRLQVGE

SEQ ID NO: 60 - protein, 16 aa, Artificial sequence
  heavy isotope-labelled peptide internal standard sequence
  MISC_FEATURE (10)..(12): U-13C6,15N2
ALDFAVGEYNKASNDM

SEQ ID NO: 61 - protein, 22 aa, Artificial sequence
  heavy isotope-labelled peptide internal standard sequence
  MISC_FEATURE (15)..(17): U-13C6,15N4
LVGGPMDASVEEEGVRRALDFA

SEQ ID NO: 62 - protein, 12 aa, Artificial sequence
  heavy isotope-labelled peptide internal standard sequence
  MISC_FEATURE (8)..(10): U-13C6,15N2
SDVMYTDWKKDK

SEQ ID NO: 63 - protein, 19 aa, Artificial sequence
  heavy isotope-labelled peptide internal standard sequence
  MISC_FEATURE (13)..(15): U-13C6,15N4
WEMPFDPQDTHQSRFYLSK

SEQ ID NO: 64 - protein, 13 aa, Artificial sequence
  heavy isotope-labelled peptide internal standard sequence
  MISC_FEATURE (7)..(9): U-13C6,15N4
EQLSLLDRFTEDA

SEQ ID NO: 65 - protein, 12 aa, Artificial sequence
  heavy isotope-labelled peptide internal standard sequence
  MISC_FEATURE (6)..(8): U-13C6,15N2
GDVAFVKHSTIF

SEQ ID NO: 66 - protein, 12 aa, Artificial sequence
  heavy isotope-labelled peptide internal standard sequence
  MISC_FEATURE (11)..(13): U-13C6,15N4
PVKWCALSHHER

SEQ ID NO: 67 - protein, 18 aa, Artificial sequence
  heavy isotope-labelled peptide internal standard sequence
  MISC_FEATURE (12)..(14): U-13C6,15N2
IAELSATAQEIIKSQAIA

SEQ ID NO: 68 - protein, 16 aa, Artificial sequence
  heavy isotope-labelled peptide internal standard sequence
  MISC_FEATURE (15)..(17): U-13C6,15N2
EAICKEQHLFLPFSYK

SEQ ID NO: 69 - protein, 14 aa, Artificial sequence
  heavy isotope-labelled peptide internal standard sequence
  MISC_FEATURE (8)..(10): U-13C6,15N4
ADQVCINLRGSFAC

SEQ ID NO: 70 - protein, 17 aa, Artificial sequence
  heavy isotope-labelled peptide internal standard sequence
  MISC_FEATURE (11)..(13): U-13C6,15N2
ILTSDVFQDCNKLVDPE

SEQ ID NO: 71 - protein, 19 aa, Artificial sequence
  heavy isotope-labelled peptide internal standard sequence
  MISC_FEATURE (13)..(15): U-13C6,15N2
YAGSQVASTSEVLKYTLFQ

SEQ ID NO: 72 - protein, 15 aa, Artificial sequence
  heavy isotope-labelled peptide internal standard sequence
  MISC_FEATURE (9)..(11): U-13C6,15N2
ESDTSYVSLKAPLTK

SEQ ID NO: 73 - protein, 15 aa, Artificial sequence
  heavy isotope-labelled peptide internal standard sequence
  MISC_FEATURE (9)..(11): U-13C6,15N2
GYSIFSYATKRQDNE

SEQ ID NO: 74 - protein, 17 aa, Artificial sequence
  heavy isotope-labelled peptide internal standard sequence
  MISC_FEATURE (11)..(13): U-13C6,15N4
STDYGIFQINSRYWCND

SEQ ID NO: 75 - protein, 12 aa, Artificial sequence
  heavy isotope-labelled peptide internal standard sequence
  MISC_FEATURE (6)..(8): U-13C6,15N2
YWCNDGKTPGAV

SEQ ID NO: 76 - protein, 12 aa, Artificial sequence
  heavy isotope-labelled peptide internal standard sequence
  MISC_FEATURE (6)..(8): U-13C6,15N4
AHVDALRTHLAP

SEQ ID NO: 77 - protein, 16 aa, Artificial sequence
  heavy isotope-labelled peptide internal standard sequence
  MISC_FEATURE (15)..(17): U-13C6,15N2
EYHAKATEHLSTLSEK

SEQ ID NO: 78 - protein, 23 aa, Artificial sequence
  heavy isotope-labelled peptide internal standard sequence
  MISC_FEATURE (20)..(22): U-13C6,15N4
TVVQPSVGAAAGPVVPPCPGRIR

SEQ ID NO: 79 - protein, 12 aa, Artificial sequence
  heavy isotope-labelled peptide internal standard sequence
  MISC_FEATURE (6)..(8): U-13C6,15N2
CNLLAEKQYGFC

SEQ ID NO: 80 - protein, 18 aa, Artificial sequence
  heavy isotope-labelled peptide internal standard sequence
  MISC_FEATURE (13)..(15): U-13C6,15N4
GGEGTGYFVDFSVRNCPR

SEQ ID NO: 81 - protein, 13 aa, Artificial sequence
  heavy isotope-labelled peptide internal standard sequence
  MISC_FEATURE (7)..(9): U-13C6,15N4
IADAHLDRVENTT

SEQ ID NO: 82 - protein, 15 aa, Artificial sequence
  heavy isotope-labelled peptide internal standard sequence
  MISC_FEATURE (9)..(11): U-13C6,15N2
VEHSDLSFSKDWSFY

SEQ ID NO: 83 - protein, 15 aa, Artificial sequence
  heavy isotope-labelled peptide internal standard sequence
  MISC_FEATURE (14)..(16): U-13C6,15N2
EYACRVNHVTLSQPK

SEQ ID NO: 84 - protein, 18 aa, Artificial sequence
  heavy isotope-labelled peptide internal standard sequence
  MISC_FEATURE (17)..(19): U-13C6,15N2
PDATKGCPDVQASLPDAK

SEQ ID NO: 85 - protein, 13 aa, Artificial sequence
  heavy isotope-labelled peptide internal standard sequence
  MISC_FEATURE (7)..(9): U-13C6,15N2
TFTLLDPKASLLT

SEQ ID NO: 86 - protein, 18 aa, Artificial sequence
  heavy isotope-labelled peptide internal standard sequence
  MISC_FEATURE (12)..(14): U-13C6,15N4
GSPAINVAVHVFRKAADD

SEQ ID NO: 87 - protein, 18 aa, Artificial sequence
  heavy isotope-labelled peptide internal standard sequence
  MISC_FEATURE (12)..(14): U-13C6,15N2
AADDTWEPFASGKTSESG

SEQ ID NO: 88 - protein, 14 aa, Artificial sequence
  heavy isotope-labelled peptide internal standard sequence
  MISC_FEATURE (13)..(15): U-13C6,15N2
GCFLKDSVTGTLPK

SEQ ID NO: 89 - protein, 19 aa, Artificial sequence
  heavy isotope-labelled peptide internal standard sequence
  MISC_FEATURE (13)..(15): U-13C6,15N4
IAYGTQGSSGYSLRLCNTG

SEQ ID NO: 90 - protein, 15 aa, Artificial sequence
  heavy isotope-labelled peptide internal standard sequence
  MISC_FEATURE (9)..(11): U-13C6,15N4
ANRPFLVFIREVPLN

SEQ ID NO: 91 - protein, 14 aa, Artificial sequence
  heavy isotope-labelled peptide internal standard sequence
  MISC_FEATURE (8)..(10): U-13C6,15N4
TSCLLFMGRVANPS

SEQ ID NO: 92 - protein, 12 aa, Artificial sequence
  heavy isotope-labelled peptide internal standard sequence
  MISC_FEATURE (11)..(13): U-13C6,15N2
QQECKHFQNLGK

SEQ ID NO: 93 - protein, 16 aa, Artificial sequence
  heavy isotope-labelled peptide internal standard sequence
  MISC_FEATURE (10)..(12): U-13C6,15N2
TINPAVDHCCKTNFAF

SEQ ID NO: 94 - protein, 15 aa, Artificial sequence
  heavy isotope-labelled peptide internal standard sequence
  MISC_FEATURE (9)..(11): U-13C6,15N4
LLDSLPSDTRLVLLN

SEQ ID NO: 95 - protein, 17 aa, Artificial sequence
  heavy isotope-labelled peptide internal standard sequence
  MISC_FEATURE (16)..(18): U-13C6,15N2
PSDTRLVLLNAIYLSAK

SEQ ID NO: 96 - protein, 14 aa, Artificial sequence
  heavy isotope-labelled peptide internal standard sequence
  MISC_FEATURE (8)..(10): U-13C6,15N2
ILNIFGVIKGFVEP

SEQ ID NO: 97 - protein, 18 aa, Artificial sequence
  heavy isotope-labelled peptide internal standard sequence
  MISC_FEATURE (12)..(14): U-13C6,15N2
VSASPLLYTLIEKTMQNV

SEQ ID NO: 98 - protein, 13 aa, Artificial sequence
  heavy isotope-labelled peptide internal standard sequence
  MISC_FEATURE (7)..(9): U-13C6,15N4
DSGSYFCRGLFGS

SEQ ID NO: 99 - protein, 11 aa, Artificial sequence
  heavy isotope-labelled peptide internal standard sequence
  MISC_FEATURE (7)..(9): U-13C6,15N4
VLDLSCNRLNR

SEQ ID NO: 100 - protein, 17 aa, Artificial sequence
  heavy isotope-labelled peptide internal standard sequence
  MISC_FEATURE (11)..(13): U-13C6,15N4
DFALQNPSAVPRFVQAI

SEQ ID NO: 101 - protein, 19 aa, Artificial sequence
  heavy isotope-labelled peptide internal standard sequence
  MISC_FEATURE (13)..(15): U-13C6,15N4
LAELPADALGPLQRAFWLD

SEQ ID NO: 102 - protein, 16 aa, Artificial sequence
  heavy isotope-labelled peptide internal standard sequence
  MISC_FEATURE (15)..(17): U-13C6,15N4
WSSLKASDTAMYYCAR

SEQ ID NO: 103 - protein, 22 aa, Artificial sequence
  heavy isotope-labelled peptide internal standard sequence
  MISC_FEATURE (16)..(18): U-13C6,15N4
VEGTAFVIFGIQDGEQRISLPE

SEQ ID NO: 104 - protein, 21 aa, Artificial sequence
  heavy isotope-labelled peptide internal standard sequence
  MISC_FEATURE (15)..(17): U-13C6,15N2
VHQYFNVELIQPGAVKVYAYY